(19) United States
(12) Patent Application Publication
(10) Pub. No.: US 2019/0083805 A1
(43) Pub. Date: Mar. 21, 2019

(54) DETECTING OR TREATING POST-TRAUMATIC STRESS SYNDROME

(71) Applicant: The Board of Trustees of the Leland Stanford Junior University, Palo Alto, CA (US)

(72) Inventor: Amit Etkin, Stanford, CA (US)

(21) Appl. No.: 16/088,291

(22) PCT Filed: Mar. 23, 2017
(86) PCT No.: PCT/US17/23857

§ 371 Date: Sep. 25, 2018

Related U.S. Application Data
(60) Provisional application No. 62/314,254, filed on Mar. 28, 2016.
(60) Provisional application No. 62/398,290, filed on Sep. 22, 2016.

(51) Int. Cl. A61N 2/00; A61B 5/0484; A61B 5/16; A61B 5/04; A61N 2/02; A61B 5/026; A61B 5/00; G01R 33/48

(52) U.S. Cl. CPC A61N 2/006; A61B 5/0484; A61B 5/165; G01R 33/48; A61N 2/02; A61B 5/0261; A61B 5/4836; A61B 5/04008

(57) ABSTRACT

The disclosures provided herewith relate to diagnosis and treatment of post-traumatic stres disorder (PTSD).

### CROSS-REFERENCE TO RELATED APPLICATIONS

This application claims the benefit of U.S. Provisional Application No. 62/314,254, filed Mar. 28, 2016, and U.S. Provisional Application No. 62/398,290, filed Sep. 22, 2016, the content of which is incorporated herein in its entirety and for all purposes.

### INCORPORATION-BY-REFERENCE OF SEQUENCE LISTING

The content of the sequence listing written in file 41243-518001WO_ST25, created on Mar. 21, 2017, 13,887 bytes in size, is hereby incorporated by reference in its entirety.

### FIELD

Provided herein relates, inter alia, to diagnosis and treatment of post-traumatic stress disorder (PTSD).

### BACKGROUND

Topological properties of human brain functional connectivity networks are thought to mediate cognition and be disrupted in neuropsychiatric disorders. Yet the nature of network/cognition relationships, their clinical relevance and molecular underpinnings remain poorly understood.

Integration and segregation are two network science principles of relevance for elucidating cognition. As applied to brain networks, integration reflects greater connection between neural subsystems and facilitates global communication of information. This allows for more distributed cognitive processing, such as executive functions or memory. Segregation reflects the partitioning of subsystems that carry out different specialized operations^{8,9}. While integration and segregation are normally inversely related, connectivity alterations in clinical populations within selective parts of a network may differentially affect measures of integration and segregation. Integration and segregation may be analyzed within specific previously-described cognition-relevant rsfMRI network modules, such as the fronto-parietal executive control (ECN), the visuospatial network (VS, also termed the dorsal attention network), the anterior insula-dorsal anterior cingulate salience network (SN) and the medial prefrontal-medial parietal-medial temporal default mode network (DMN).

Brain regions that form functionally connected large-scale networks have more highly correlated patterns of gene expression than regions outside these networks. Different regions even within the same network play different roles (e.g. hubs versus non-hubs). Molecular mechanism may be revealed through the expression of candidate genes whether discovered through single nucleotide polymorphism (SNP) or genome-wide association studies.

Complicating this understanding of post-traumatic stress disorder (PTSD) neurocircuitry, however, is the disorder's substantial heterogeneity. Current Diagnostic and Statistical Manual (DSM) symptom-based criteria can yield a PTSD diagnosis in different ways, which furthermore have only modest diagnostic overlap with previous DSM-IV definitions of PTSD.

Cognitive dysfunction is a core feature of PTSD. Like many major psychiatric disorders, PTSD has well-documented impairments in memory, attention, and speed of information processing. Yet the neural mechanisms underlying cognitive deficits, their relationships to treatment outcome, and potential utility from a biomarker perspective are largely unknown. Specific configurations of brain networks (i.e. network topology) may underlie different aspects of cognition-relevant information processing. Resting-state functional magnetic resonance (rsfMRI) studies in PTSD have demonstrated abnormal connectivity in various cognition-relevant networks, but there is no established relation with network topology or behavior. Furthermore, there can be some diagnostic confusion between PTSD and traumatic brain injury (TBI) or other conditions especially as these conditions may be comorbid or occur at higher rates in combat veterans.

Accordingly, there is a need in the field to have more consistent and effective criteria for not only identification and diagnosis of PTSD, but also for tailoring of treatment strategies for best therapeutic outcome. Provided herein are solutions for these and other problems in the art.

### SUMMARY

Provided herein, inter alia, are methods and compositions based on the surprising discovery that abnormal connectivity in various cognition-relevant networks occurs in patients with PTSD. The systems and methods provided herein may be particularly valuable for revealing the connectomic basis of cognitive deficits in PTSD having utility in diagnosis, prognosis, and determination of treatment strategy.

In one aspect, provided herein is a method of treating post-traumatic stress disorder in a subject in need thereof. The method including (i) determining connectivity between a first cognitive region within the brain of the subject and a second cognitive region within the brain of the subject or determining a complex cognitive behavioral deficiency in the subject; and (ii) administering a post-traumatic stress disorder treatment to the subject. In embodiments, the method includes determining connectivity between a first cognitive region within the brain of the subject and a second cognitive region within the brain of the subject. In embodiments, the first cognitive region and the second cognitive region are independently selected from the group consisting of left middle frontal gyrus, left inferior frontal gyrus, left inferior parietal lobule, left middle temporal gyrus, left thalamus, right middle frontal gyrus, right inferior frontal gyrus, right inferior parietal lobule, right dorsomedial PFC, left lateral cerebellum, right caudate, left anterior middle frontal gyrus, left insula, dorsal anterior cingulate cortex (ACC), right anterior middle frontal gyrus, right insula, left lateral cerebellum, right lateral cerebellum, left frontal eye fields, left intraparietal sulcus, left inferior frontal cortex, left inferior temporal gyrus, right frontal eye fields, right intraparietal sulcus, right inferior frontal cortex, right inferior temporal gyrus, right lateral cerebellum, medial prefrontal cortex, left angular gyrus, right superior frontal gyrus, posterior cingulate gyms, mid-cingulate gyrus, right angular gyrus, thalamus, left hippocampus, and right hippocampus; wherein the first cognitive region is different from the second cognitive region.

In embodiments, the determining connectivity is performed using a functional connectivity analysis. In embodiments, the functional connectivity analysis is a blood flow analysis. In embodiments, the blood flow analysis is an fMRI analysis. In embodiments, the blood flow analysis is a near infrared spectroscopy (NIRS) analysis. In embodiments, the functional connectivity analysis is an electroencephalogram (EEG) analysis. In embodiments, functional connectivity analysis is a magnetoencephalography (MEG) analysis.

In embodiments, the determining connectivity includes administering a transcranial magnetic stimulation (TMS) thereby producing an evoked response. In embodiments, the TMS is administered to the right executive control network (ECN). In embodiments, the evoked response is monitored by Electroencephalography (EEG). In embodiments, an amplitude of the evoked response is measured by EEG at about 30-250 ms after stimulation.

In embodiments, the determining connectivity is performed using a structural connectivity analysis. In embodiments, the structural connectivity analysis is a diffusion-weighted structural connectivity analysis. In embodiments, the method further includes determining a complex cognitive behavioral deficiency in the subject. In embodiments, the complex cognitive behavioral deficiency is a memory deficiency. In embodiments, the memory deficiency is a long term memory deficiency, a working memory deficiency, a short term memory deficiency, a delayed recall deficiency or an immediate recall deficiency. In embodiments, the determining is performed using a card sorting analysis, reward or punishment learning tests, planning test, or navigation test.

In embodiments, the post-traumatic stress disorder treatment includes psychotherapy. In embodiments, the treatment includes repetitive transcranial magnetic stimulation (rTMS). In embodiments, the rTMS is administered to the right executive control network (ECN). In embodiments, the psychotherapy is selected from the group consisting of prolonged exposure therapy, cognitive processing therapy, cognitive behavioral therapy, eye movement and desensitization therapy, acceptance and commitment therapy, and interpersonal psychotherapy.

In another aspect, provided herein is a method of determining connectivity between cognitive regions in a patient suffering from or suspected of suffering from a post-traumatic stress disorder, the method includes determining connectivity between a first cognitive region within the brain of the subject and a second cognitive region within the brain of the subject or determining a complex cognitive behavioral deficiency in the subject. In embodiments, the patient is undergoing a course of treatment for a post-traumatic stress disorder.

In another aspect, provided herein is a system including, at least one processor; and at least one memory including program code which when executed by the at least one memory provides operations including, determining a connectivity between a first cognitive region and a second cognitive region within a brain of a subject and/or determining a complex cognitive behavioral deficiency in the subject; and providing, via a user interface, a post-traumatic stress disorder treatment plan for the subject. In embodiments, the connectivity between the first cognitive region and the second cognitive region within the brain of the subject and the complex cognitive behavioral deficiency in the subject includes a biomarker associated with the subject. In embodiments, the system further includes determining a presence of the biomarker in the subject by at least: screening the subject for memory deficit; and performing, on the brain of the subject, an imaging test, when a result of the screening indicates that the subject exhibits memory deficit. In embodiments, the imaging test includes functional magnetic resonance imaging (fMRI) and/or magnetoencephalography (MEG) or electroencephalograpy (EEG).

In embodiments, the system further includes determining, based on a presence of the biomarker in the subject, the post-traumatic stress disorder treatment plan for the subject. In embodiments, psychotherapy is excluded from the post-traumatic stress disorder treatment plan for the subject, when the biomarker is determined to be present in the subject. In embodiments, psychotherapy is included in the post-traumatic stress disorder treatment plan for the subject, when the biomarker is determined to be not present in the subject. In embodiments, non-invasive brain stimulation is included in the post-traumatic stress disorder treatment plan for the subject, when the biomarker is determined to be present in the subject. In embodiments, the treatment plan further includes medication and/or non-invasive brain stimulation. In embodiments, the non-invasive brain stimulation is administered based at least on the connectivity between the first cognitive region and the second cognitive region within the brain of the subject. In embodiments, determining the connectivity between the first cognitive region and the second cognitive region within the brain of the subject includes evoking a response by administering transcranial magnetic stimulation (TMS).

In embodiments, TMS is administered to a right executive control network (ECN). In embodiments, the system further includes monitoring, via Electroencephalography (EEG), the response evoked by the administration of the TMS. In embodiments, the monitoring of the response evoked by the administration of the TMS includes measuring, by EEG, an amplitude of the response approximately 30-250 milliseconds (ms) subsequent to the administration of TMS. In embodiments, the system is further configured to perform operations including the method as recited above.

In a further aspect, provided herein is a non-transitory computer-readable storage medium including program code which when executed by at least one processor causes operations including: determining a connectivity between a first cognitive region and a second cognitive region within a brain of a subject and/or determining a complex cognitive behavioral deficiency in the subject; and providing, via a user interface, a post-traumatic stress disorder treatment plan for the subject. In embodiments, the connectivity between the first cognitive region and the second cognitive region within the brain of the subject and the complex cognitive behavioral deficiency in the subject includes a biomarker associated with the subject. In embodiments, the computer-readable storage medium further includes determining a presence of the biomarker in the subject by at least: screening the subject for memory deficit; and performing, on the brain of the subject, an imaging test, when a result of the screening indicates that the subject exhibits memory deficit. In embodiments, the imaging test includes functional magnetic resonance imaging (fMRI) and/or magnetoencephalography (MEG). In embodiments, the computer-readable storage medium further includes determining, based on a presence of the biomarker in the subject, the post-traumatic stress disorder treatment plan for the subject. In embodiments, psychotherapy is excluded from the post-traumatic stress disorder treatment plan for the subject, when the biomarker is determined to be present in the subject. In embodiments, psychotherapy is included in the post-traumatic stress disorder treatment plan for the subject, when the biomarker is determined to be not present in the subject.

In embodiments, the treatment plan further includes medication and/or non-invasive brain stimulation. In embodiments, the non-invasive brain stimulation is administered based at least on the connectivity between the first cognitive region and the second cognitive region within the brain of the subject. In embodiments, the connectivity between the first cognitive region and the second cognitive region within the brain of the subject includes evoking a response by administering transcranial magnetic stimulation (TMS). In embodiments, the TMS is administered to a right executive control network (ECN). In embodiments, the computer-readable storage medium further includes monitoring, via Electroencephalography (EEG), the response evoked by the administration of the TMS. In embodiments, the monitoring of the response evoked by the administration of the TMS includes measuring, by EEG, an amplitude of the response approximately 30-250 milliseconds (ms or msec) subsequent to the administration of TMS. In embodiments, the operations further includes the method as recited above.

In a further aspect, provided herein is an apparatus including means for determining a connectivity between a first cognitive region and a second cognitive region within a brain of a subject and/or means for determining a complex cognitive behavioral deficiency in the subject; and means for providing a post-traumatic stress disorder treatment plan for the subject. In embodiments, the connectivity between the first cognitive region and the second cognitive region within the brain of the subject and the complex cognitive behavioral deficiency in the subject includes a biomarker associated with the subject. In embodiments, the apparatus is further configured to determine a presence of the biomarker in the subject by at least: screening the subject for memory deficit; and performing, on the brain of the subject, an imaging test, when a result of the screening indicates that the subject exhibits memory deficit. In embodiments, the imaging test includes functional magnetic resonance imaging (fMRI) and/or magnetoencephalography (MEG) or electroencephalography (EEG).

In embodiments, the apparatus is further configured to determine, based on a presence of the biomarker in the subject, the post-traumatic stress disorder treatment plan for the subject. In embodiments, the apparatus is configured to exclude psychotherapy from the post-traumatic stress disorder treatment plan for the subject, when the biomarker is determined to be present in the subject. In embodiments, the apparatus is configured to include psychotherapy in the post-traumatic stress disorder treatment plan for the subject, when the biomarker is determined to be not present in the subject. In embodiments, the treatment plan further includes medication and/or non-invasive brain stimulation. In embodiments, the non-invasive brain stimulation is administered based at least on the connectivity between the first cognitive region and the second cognitive region within the brain of the subject. In embodiments, the apparatus is configured to determine the connectivity between the first cognitive region and the second cognitive region within the brain of the subject by at least evoking a response by administering transcranial magnetic stimulation (TMS). In embodiments, the TMS is administered to a right executive control network (ECN). In embodiments, the apparatus is further configured to monitor, via Electroencephalography (EEG), the response evoked by the administration of the TMS. In embodiments, the apparatus is configured to monitor the response evoked by the administration of the TMS by at least measuring, by EEG, an amplitude of the response approximately 30-250 milliseconds (ms) subsequent to the administration of TMS. In embodiments, the apparatus further includes means for performing the method as recited above.

In another aspect, provided herein is a method of treating post-traumatic stress disorder in a subject in need thereof, the method including: determining a connectivity between a first cognitive region within a brain of the subject and a second cognitive region within the brain of the subject and/or determining a complex cognitive behavioral deficiency in the subject; and providing, via a user interface, a post-traumatic stress disorder treatment plan for the subject. In embodiments, the connectivity between the first cognitive region and the second cognitive region within the brain of the subject and the complex cognitive behavioral deficiency in the subject includes a biomarker associated with the subject. In embodiments, the method further includes determining a presence of the biomarker in the subject by at least: screening the subject for memory deficit; and performing, on the brain of the subject, an imaging test, when a result of the screening indicates that the subject exhibits memory deficit. In embodiments, the imaging test includes functional magnetic resonance imaging (fMRI) and/or magnetoencephalography (MEG) or electroencephalography (EEG).

In embodiments, the method further includes determining, based on a presence of the biomarker in the subject, the post-traumatic stress disorder treatment plan for the subject. In embodiments, psychotherapy is excluded from the post-traumatic stress disorder treatment plan for the subject, when the biomarker is determined to be present in the subject. In embodiments, psychotherapy is included in the post-traumatic stress disorder treatment plan for the subject, when the biomarker is determined to be not present in the subject. In embodiments, the treatment plan further includes medication and/or non-invasive brain stimulation. In embodiments, the non-invasive brain stimulation is administered based at least on the connectivity between the first cognitive region and the second cognitive region within the brain of the subject. In embodiments, the connectivity between the first cognitive region and the second cognitive region within the brain of the subject includes evoking a response by administering transcranial magnetic stimulation (TMS).

In embodiments, the TMS is administered to a right executive control network (ECN). In embodiments, the method further includes monitoring, via Electroencephalography (EEG), the response evoked by the administration of the TMS. In embodiments, the monitoring of the response evoked by the administration of the TMS includes measuring, by EEG, an amplitude of the response approximately 30-250 milliseconds (ms) subsequent to the administration of TMS. In embodiments, the method as recited above.

In another aspect, provided herein is a method of treating a subject having or suspected of having inhibitory right ECN post-traumatic stress disorder, the method including administering TMS. In embodiments, the TMS is administered to the right ECN. In embodiments, TMS is delivered repetitively in a pattern that is intended to induce plasticity. In embodiments, TMS is rTMS. In embodiments, rTMS includes stimulation at greater than 5 Hz. In embodiments, rTMS includes stimulation at less than or equal to 1 Hz. In embodiments, rTMS includes either a continuous or an intermittent theta burst pattern.

### BRIEF DESCRIPTION OF THE DRAWINGS

FIG. 1A-1C demonstrate behavioral task performance in Study 1. FIG. 1A is a graph showing reduced delayed verbal memory recall in a word learning task in PTSD on delayed recall (Mann-Whitney U=664, p=0.043) but no difference during learning (U=722.5, p=0.171). FIG. 1B is a graph showing slower reaction times RTs (unpaired t=1.96, p=0.05) and lower accuracies (U=636, p=0.049) on a continuous performance task (CPT) in PTSD. FIG. 1C is a graph showing a trend towards slower reaction times on a choice RT task in PTSD (t=1.81, p=0.074). * p<0.05, † p<0.1, shown are medians and interquartile ranges.

FIG. 2A-2E show network segregation and integration in Study 1. FIG. 2A is a series of schematic drawings showing regions of interest (only cortical shown) for the four cognitive control networks (DMN: default mode network, SN: salience network, VS: visuospatial network, ECN: executive control network). FIGS. 2B and 2C are graphs showing lower average network efficiency (FIG. 2B) (F=4.19, p=0.044) and network segregation (system segregation index) (FIG. 2C) in patients with PTSD (F=6.45, p=0.013). * p<0.05, † p<0.1, shown are mean and standard errors. Maximum root mean square motion, a potential confounding factor in rsfMRI analyses, did not differ between groups (F=0.32, p=0.571) nor did it correlate with efficiency or system segregation (r's<−0.122, p's>0.238). There was no interaction between group and threshold for efficiency or segregation. FIG. 2D is a graphic of a minimum spanning tree (MST). Connections show the group difference in connectivity strength for each pair of nodes (r-to-z transformed), expressed using Cohen's d, with no threshold applied. Red=controls>patients, blue=patients>controls. The plot is divided into within and between-network connectivity for display clarity. Connection thickness and color scale with magnitude of Cohen's d, with those connections with the greatest difference displayed on top of the weaker connections. See full names of each node in Table 2. FIG. 2E is a graphic summary across all relevant network connections of the group difference (Cohen's d) in unthresholded connectivity strengths shown in D. Reduced overall connectivity helped account for decreased network efficiency in patients, as evident by a non-significant group difference in efficiency when controlling for average connectivity (F=2.26, p=0.137), but not when doing the same for system segregation (F=8.07, p=0.006). Hypoconnectivity was seen within all networks, most striking for the VS network. This was quantified by comparing within- and between network connectivity strength, and finding a group x within/between-network interaction (F=4.13, p=0.045). Average network connectivity strength also related strongly to network efficiency (r=0.944, p<0.001), but did not relate to system segregation (r=−0.066, p=0.524). Instead, system segregation was related to the disproportionate loss of within-network connectivity (within divided by between-network ratio r=0.616, p<0.001), which was in turn not related to network efficiency (r=0.009, p=0.927).

FIG. 3A-3E indicate a relationship between network topology and verbal memory impairment in Study 1. FIG. 3A is a graph showing patients with impaired verbal memory delayed recall, compared to either patients with intact memory or healthy controls, had lower network efficiency (F=3.96, p=0.023). Since memory recall had maximal values of 100% and thus negatively skewed, a median (≥ or <95% accuracy) split was used to divide patients into an impaired group (relative to healthy participants) and an intact group (see Methods). Consistent with expectations of the median splits, treating memory recall in this way resulted in an impaired patient group that differed from both the intact patient group and healthy participants (U's<13.5, p's<0.001) but the latter two groups did not differ from each other (U=636.5, p=0.927).). Efficiency was significantly lower for memory-impaired patients than either intact memory patients (t=2.04, p=0.046) or healthy participants (t=3.12, p=0.003), but no difference between intact patients and healthy participants (t=0.95, p=0.344) FIG. 3B is a graph showing there was a trend effect of group on system segregation (F=2.82, p=0.065), but this was driven by significantly greater segregation in healthy participants than intact patients (t=2.15, p=0.035) and a similar trend versus impaired patients (t=1.65, p=0.119) and no difference between the two patient groups (t=0.47, p=0.638). FIG. 3C is a graph showing there was a significant effect of group on average network connectivity strength (F=3.83, p=0.025), but this was driven by significantly greater connectivity in controls than impaired patients (t=2.87, p=0.006), a similar trend between controls and intact patients (t=1.92, p=0.059) but no difference between impaired and intact patients (t=1.28, p=0.209). FIG. 3D is a graph showing average closeness. Using the memory-based grouping, an effect of group on average closeness was found across nodes within the ECN, SN and DMN networks (F<4.69, p's<0.012; FIG. 5A), and a trend for the VS network (F=3.06, p=0.052). The significant group effects were driven by lower closeness in the ECN, SN and DMN in memory impaired patients compared to intact patients (t>2.9, p's<0.006) or healthy controls (t>2.79, p's<0.008) and no difference between the two patient groups (t<1.21, p's>0.232). FIG. 3E is a schematic showing regions surviving FDR correction for the relationship of closeness to memory impairment (right insula trend at FDR q=0.054). * p<0.05, † p<0.1, shown are means and standard errors.

FIG. 4A-4C show replication of network-cognition relationships in Study 2 and its biomarker characteristics. FIG. 4A is a graph showing an effect of memory-based grouping on efficiency was found (F=4.54, p=0.014), driven by decreased efficiency in impaired memory PTSD participants compared to either intact memory PTSD or healthy participants (F's>6.24, p's<0.017), with the latter two groups not differing from each other (F=0.13, p=0.720). Impaired PTSD patients had significantly lower efficiency than either TBI subgroups (F's>5.47, p's<0.022) and the TBI subgroups did not differ from each other (F=0.46, p=0.633). * p<0.05, shown are means and standard errors. Total root mean square motion did not differ between groups (F=0.66, p=0.523). FIG. 4B is a receiver-operating characteristic (ROC) curve differentiating efficiency in healthy participants from that in memory impaired PTSD patients in study 1 (AUC=0.77, p<0.001) to determine an optimal efficiency cutpoint to pair with impaired delayed recall to define a combined efficiency/memory biomarker. FIG. 4C is a table showing the application of this independently derived combined biomarker to participants in Study 2, in which PTSD are identified at a greater rate than both healthy participants (χ2=5.14, p=0.023) and TBI patients (χ2=12.64, p<0.001). Against these groups, the biomarker has 94-95% specificity.

FIG. 5A-5B show prediction of psychotherapy treatment outcome in PTSD by network efficiency and verbal memory. FIG. 5A is a plot showing a significant interaction between treatment arm (prolonged exposure versus wait list)×memory (impaired versus intact)×efficiency (as a continuous variable)×time (F=7.17, p=0.009), which is visualized using a median split of efficiency and impaired/intact memory split as in prior figures. FIG. 5B is a plot showing similar results when using the combined efficiency/memory biomarker from FIG. 4A-4C (arm×biomarker×time interaction F=4.37, p=0.04). Here, patients positive on this cognitive dysfunction biomarker saw no benefit of exposure therapy over wait list (arm×time interaction F=1.0, p=0.330; treatment effect size d=0.6, wait-list d=0.4), while patients negative on the biomarker saw a robust advantage of exposure therapy over wait list (arm×time interaction F=83.22, p<0.001; treatment effect size d=3.5, wait-list d=0.5).

FIG. 6A-6C show potential molecular mechanisms for network integration. FIG. 6A is a plot of correlations between regional expression of the CRHR1 gene and those regions' closeness centrality in six post-mortem healthy individuals. Shown are correlations, with points color-coded by donor (each dot represents a different region), for each of the AIBS donor datasets. Gene expression values (x-axis) allow for correlations across regions within donor and not normalized across donors. Hence, correlation coefficients and p-values were determined separately for each donor, and then combined across donors using Fisher's method (for the correlation coefficient) and Stouffer's method (yielding a one-sided p-value). An FDR correction was applied across all PTSD-related genes. Greater CRHR1 expression was associated with lower closeness centrality across network nodes (r=−0.51, FDR q=0.0007; FIG. 6). FIG. 6B is a plot indicating greater STMN1 expression was associated with lower closeness (r=−0.39, FDR q=0.009). The correlation between CRHR1 expression and closeness centrality remained significant even if controlling for nodal participation coefficient as a measure of segregation (r=−0.43, p=0.0006), and similarly for STMN1 (r=−0.39, p=0.001). FIG. 6C is a graph showing the CRHR1 risk allele (GG homozygotes at rs110402) was associated with lower network efficiency, after controlling for scanner, and presence of comorbid depression and PTSD (F=4.02, p=0.047).

FIG. 7A-7B show within- and between-network connectivity for the healthy versus memory-impaired PTSD patients (FIG. 7A) and intact versus impaired PTSD patients (FIG. 7B) in Study 1. Shown is the group difference in connectivity strength for each pair of nodes (r-to-z transformed), expressed using Cohen's d, with no threshold applied. Red=healthy or intact>impaired, blue=impaired>healthy or intact.

FIG. 8A-8B show within- and between-network connectivity for the healthy versus memory-impaired PTSD patients (FIG. 7A) and intact versus impaired PTSD patients (FIG. 7B) in Study 2. Shown is the group difference in connectivity strength for each pair of nodes (r-to-z transformed), expressed using Cohen's d, with no threshold applied. Red=healthy or intact>impaired, blue=impaired>healthy or intact.

FIG. 9A-9B show that no change in efficiency (F=1.03, p=0.313) (FIG. 9A) or verbal memory delayed recall (F=0.94, p=0.337) (FIG. 9B) as a function of prolonged exposure (PE) treatment or a wait-list (WL) minimal attention intervention. Means and standard errors (efficiency) or medians and interquartile ranges (memory) are shown by group before and after treatment.

FIG. 10 is a system diagram illustrating a system for treating post-traumatic stress disorder, in accordance with some example embodiments.

FIG. 11 is a flowchart illustrating a process for treating post-traumatic stress disorder, in accordance with some example embodiments.

FIG. 12A-C show causal connectomic mapping of intrinsic network deficits using concurrent spTMS/EEG. FIG. 12A Middle top is an example TMS-evoked potential drawn from right frontal electrodes after right ECN spTMS stimulation, illustrating the peaks quantified for analysis (prior to rectification). FIG. 12A Middle bottom is an image showing prefrontal targets for the ECN and SN located within the middle frontal gyrus, as localized by an independent components analysis on separate resting fMRI data. An omnibus test on the amplitude of rectified potentials revealed a TMS site×potential×group interaction using the memory-based grouping (generalized linear model χ²=160, p<0.001). Splitting by potentials revealed group by site interactions for the p60 (χ²=15.02, p<0.02) and n100 potentials (χ²=13.14, p=0.04). A group x site n100 interaction, which was not further moderated by electrode cluster (χ²=9.71, p=0.466) was driven by right ECN TMS stimulation (bar graphs). Rectified potentials averaged across electrode clusters showed significantly greater n100 responses to right ECN TMS stimulation only in memory-impaired PTSD participants relative to either the healthy or memory-intact PTSD participants (right ECN TMS group effect χ²=9.25, p=0.01; post-hoc impaired (n=10) vs. healthy (n=12) p=0.014, impaired vs. intact (n=15) p=0.003, intact vs. healthy p=0.896; bottom right). Group effects were not significant for the other TMS sites. FIG. 12B is a scalp topography plot of the rectified n100 response to ECN TMS in each of the three groups, illustrating a stronger n100 particularly in fronto-central electrodes in memory-impaired PTSD participants. FIG. 12C is a graph showing that greater resting fMRI-determined network efficiency correlated negatively with the amplitude of rectified n100 potentials in response to right ECN TMS stimulation in PTSD participants (χ²=7.46, p=0.006), such that those with the largest n100s had the lowest global efficiency (controlling for age). * p<0.05, ** p<0.01. Shown are means and standard errors.

FIG. 13 is a diagram showing EEG electrode cluster locations.

FIG. 14 demonstrates reduction of psychotherapy treatment outcome in PTSD by network efficiency and verbal memory in Study 1. PTSD patients (n=66, see supplemental FIG. 1 for CONSORT diagram) were randomized to receive psychotherapy treatment (prolonged exposure) or a minimum attention wait-list control for the same period. FIG. 14 is a line plot showing that in an intent-to-treat linear mixed model analysis, an interaction between treatment arm (prolonged exposure versus wait list)×time (F(2,113)=20.05, p<0.001), was observed due to greater symptom improvement with prolonged exposure (post-hoc effect of time: p<0.001) than wait-list (p=0.036). To test whether the network/memory phenotypes moderates this difference in outcome, memory and efficiency were entered into the model and found a significant interaction between treatment arm×memory (impaired versus intact)×global efficiency (continuous)×time (F(2,81)=15.78, p<0.001). The figure visualizes this interaction by dividing patients in each arm by the binary memory impairment cutoff and dividing continuous global efficiency values using the cutoff that differentiates between memory-impaired and memory-intact PTSD participants. As such, the patients with both impaired memory and efficiency are those with the combined network/memory phenotype biomarker described earlier. Moderation analysis using presence of this biomarker also yielded a significant treatment arm×biomarker×time interaction (F(2,89)=11.59, p<0.001). Both interactions are driven by a profound lack of response in patients with the network/memory-impairment phenotype to exposure therapy vs. wait list (Cohen's d=−0.6). By contrast, patients without this phenotype respond robustly to therapy vs. wait-list (d=6.4), with many reaching remission (CAPS≤20). Importantly, neither verbal memory (F(2,90)=2.04, p=0.136) nor network efficiency (F(2,105)=0.812, p=0.447) moderated treatment outcome when considered alone. Motion also did not moderate treatment outcome (group×time×motion: F(2,106)=0.36, p=0.701), nor was it affected by either intervention (group×time: F(2,110)=2.0, p=0.141).

FIG. 15 is a CONSORT diagram for the treatment component.

FIGS. 16A and B demonstrate a method for assessing EEG connectivity according to an embodiment.

### DETAILED DESCRIPTION OF CERTAIN EMBODIMENTS

In some aspects, provided herein, inter alia, systems and methods for the detection, diagnosis and identification of appropriate treatment for PTSD. In embodiments, the systems and methods utilize mathematical modelling based to assess brain region connectivity. Measures of connectivity based on functional readouts of neural activity may include, for example, MRI and EEG, as well as cognitive and behavioral tasks that can be used in the diagnosis and treatment evaluation of PTSD. Furthermore, systems and methods provided herein can also distinguish PTSD symptoms from other disorders including traumatic brain injury (TBI). Genetic markers indicators of PTSD are also disclosed herein.

Also described herein are systems and methods for elucidating the integration/segregation abnormalities in PTSD that would reflect patients' cognitive abnormalities, which would further predict treatment outcome and be a useful biomarker to distinguish PTSD from healthy and TBI individuals. Specific PTSD-associated genes whose expression related to cognition-relevant network topology are also identified.

### Definitions

As used herein, the term “cognitive network” or “cognitive module” designates a grouping of cognitive regions or nodes that support associated cognitive processes. Example cognitive networks include the frontoparietal network (also called the executive control, central executive, or attentional network), the dorsal attention network (also called the visuospatial or spatial attention network), the salience network (also called the ventral attention or cingulo-opercular network) and the default mode network. Each cognitive network may comprise a number of cognitive nodes or cognitive regions, identifiable by, for example, independent component analysis (ICA).

As used herein, the term “cognitive region” or “cognitive node” is a continuous physical portion of the brain (e.g. cerebral cortex, hippocampus, thalamus or cerebellum) that supports a cognitive process. Cognitive regions may include, for example a gyrus, a sulcus or an area covering a collection of gyri or sulci. Cognitive regions may be grouped by associated function, activity, or connectivity into cognitive networks (also called cognitive modules).

As used herein, the term “connectivity” in relation to one or more cognitive regions refers to anatomical connectivity, functional connectivity, or causal connectivity between cognitive regions. Anatomical (or structural) connectivity includes intact structural links such as neuronal, synaptic or fiber pathways. Functional connectivity includes simultaneous or near simultaneous change in activity (e.g. less than 1 second when read via electrical stimulus, or on a time scale of several seconds when viewed by changes in blood flow, for example as analyzed by fMRI) between cognitive regions. The change in activity may be an increase or decrease from an average level activity. Functional connectivity, therefore, includes phasic relationships or waveform activity between cognitive regions. Causal connectivity is related to functional activity in that it is a response evoked in one region in response to stimulation of another region. Examples of methods of assaying neural activity include blood flow analysis (e.g. fMRI, or near infrared spectroscopy (NIRS)), functional connectivity analysis (e.g. electroencephalogram (EEG) or magnetoencephalography (MEG)), or structural connectivity analysis (e.g. diffusion-weighted structural connectivity analysis).

As used herein, the term “complex cognitive behavioral deficiency” refers to any degree of abnormality observed or present when performing one or more cognitive and/or behavioral tasks (e.g., memory, word learning, continuous performance, and choice reaction time).

As used herein, the term “a blood flow analysis” refers to any type of assays or tests that can detect changes in blood flow, for example, in any part or entire brain.

As used herein, the term “functional magnetic resonance imaging or functional MRI (fMRI)” refers to a functional neuroimaging procedure using MRI technology that measures brain activity by detecting changes associated with blood flow.

As used herein, the term “near-infrared spectroscopy (NIRS)” refers to a spectroscopic method that uses the near-infrared region of the electromagnetic spectrum (from about 700 nm to 2500 nm). For example, NIRS can be used for non-invasive assessment of brain function through the intact skull in human subjects by detecting changes in blood hemoglobin concentrations associated with neural activity, e.g., in branches of cognitive psychology.

As used herein, the term “electroencephalography (EEG)” refers to a neurological test that uses an electronic monitoring device to measure and record electrical activity in the brain.

As used herein, the term “magnetoencephalography (MEG)” refers to a non-invasive neurophysiological technique that measures the magnetic fields generated by neuronal activity of the brain. The spatial distributions of the magnetic fields are analyzed to localize the sources of the activity within the brain.

As used herein, the term “diffusion-weighted structural connectivity analysis” refers to an imaging method that uses the diffusion of water molecules to generate contrast in MR images. It allows the mapping of the diffusion process of molecules, e.g. water, in biological tissues, in vivo and non-invasively.

The signaling in a biological neural network is based on a highly coordinated system of electric charges, neurotransmitters and action potentials. The ability to reliably and non-invasively incite and monitor neuronal activity changes from outside the head with the purpose of modulating activity in specific neural networks remains a roadblock to enable advances in the detection, monitoring, and treatment of psychiatric, neurological and related conditions. A neural network can be considered as a complex electrical circuit made of many neurons connected through synapses formed between axons and dendrites. Both types of synapses, known as chemical and electrical synapses, respectively, transfer information between adjacent axons and dendrites directly or indirectly through electric field energy. Consequently, the neural network is sensitive to external electric fields. Existing non-invasive brain stimulation methods include transcranial magnetic stimulation (TMS), transcranial direct current stimulation (tDCS) and transcranial alternating current stimulation (tACS).

Non-invasive brain stimulation locally alters brain electrical signaling. These local alterations in signaling can result in broader alterations to neuronal signaling throughout the brain. These circuit-wide effects of non-invasive brain stimulation reflect the brain effects of stimulation as well as the network rebound response to a burst of activity entering the system. This set of events is referred to herein as a non-invasive brain stimulation evoked response (e.g., a TMS evoked response).

In embodiments, a magnitude of a non-invasive brain stimulation evoked response is measured at 25-50 msecs, 100-150 msecs, or 180 and 200 msec following non-invasive brain stimulation. The TMS evoked response can be measured between 25-50 msecs (p30), 30-70 msecs (p60), 70-120 msecs (n100), 150-250 msecs (p200). Alternatively, the TMS evoked response can be measured on the amplitude of oscillations at theta (5-8 Hz), alpha (8-12 Hz), beta (12-30 Hz), or gamma (30-60 Hz) within the first second after a TMS pulse.

In embodiments, an evoked response is an electrical potential recorded from the nervous system, e.g. brain, of a human or other animal following presentation of a stimulus, as distinct from spontaneous potentials as detected by electroencephalography (EEG), electromyography (EMG), or other electrophysiologic recording method. Such potentials are useful for electrodiagnosis and monitoring. The recorded electrical potential is often presented with an amplitude, phase and/or frequency which generally indicates an intensity and/or patent of the response.

As used herein, the term “memory deficiency” refers to performance of patients below levels observed in healthy individuals in a test of memory. In some embodiments, memory deficiency may be a long term memory deficiency (which refers to impairments in the recall of previously learned memories or associations), a working memory deficiency (which refers to impairments in the ability to hold multiple pieces of information in mind), a short term memory deficiency (which refers to impairments in recall of information learned within several minutes of initial learning), a delayed recall deficiency (which refers to impairments in recall of information learned >about 10 minutes prior) or an immediate recall deficiency (which refers to impairments in the ability to recall information immediately after it was learned).

As used herein, the term “connectivity efficiency” refers to the degree of connectivity between two or more cognitive regions (e.g. 10 or more, 20 or more, or 30 or more cognitive regions). In one example, connectivity efficiency may be a measure of functional and/or anatomical connectivity. In another example, connectivity efficiency may approximate direct anatomical connectivity via synaptic connection. These different types of connectivity efficiency may be, but are not necessarily, interrelated. For example, closely correlated activity amongst two brain nodes may not be indicative of a direct anatomical connection.

Connectivity efficiency can be quantified by applying graph theory to measures of neural activity. Mathematical models derived from graph theory allow for calculation of metrics use to quantify connectivity efficiency including “global efficiency”, “system segregation”, “participation coefficient” and “closeness centrality”.

As used herein “global efficiency” is a measure of connectivity efficiency aimed at assessing overall connectivity by averaging efficiency over all cognitive regions (also referred to here as “network nodes,” “networks” or “nodes”). A network node's connectivity efficiency may be defined as the mean of the inverse-shortest-distance from that node to all other nodes. The global efficiency can be expressed as,

$${E}_{Global} = \frac{1}{n} {\sum }_{i ∈ N} \frac{{\sum }_{j ∈ N , i \neq j} \frac{1}{{d}_{ij}}}{n - 1 \\} , \qquad ( 1 )$$

where dᵢⱼ is the shortest distance between the i^{th} and j^{th} nodes, n is the number of nodes in the graph. Distances between two nodes are the inverse of their correlations, and the shortest distances were calculated using BGL's implementation of the Dijkstra's algorithm ²².

As used herein, “system segregation” defines the community structure of a network, in particular, segregation is a measure of how well the networks follow a priori community structure defined by independent component analysis (ICA). System segregation is defined as the difference between the mean within-module and between-module connectivity relative to the mean within-module connectivity¹. System segregation was calculated using,

$$S = \frac{{\overline{Z}}_{W} - {\overline{Z}}_{B}}{{\overline{Z}}_{W}} \qquad ( 2 )$$

where S is the system segregation measure, Z̅_{W} is the mean within-module connectivity and Z̅_{B} is the mean between-module connectivity. Z̅_{W} and Z̅_{B} are calculated by, respectively, averaging all the within-module and between module edge weights. The edge weights are Fisher's z-transformed Pearson's correlation estimates between each region.

As used herein “participation coefficient” is a quantification of the degree to which a node serves as a communication hub between nodes outside its module and those within its module. The module definitions were implicit to the node definitions as they were derived from the ICA maps. Participation coefficient can be expressed as follows,

$${P}_{coeff} = 1 - {\sum }_{m ∈ M} {( \frac{{k}_{i} ( m )}{{k}_{i}} )}^{2} , \qquad ( 3 )$$

where M is the set of modules, kᵢ is the node degree and kᵢ(m) is the total connection strength from node i to module m.

As used herein “closeness centrality” is defined for each node as inverse of the average shortest-distance. Closeness centrality, which is very tightly related to efficiency, is defined below.

$$CC = \frac{1}{L} = \frac{n - 1}{{\sum }_{j ∈ N , i \neq j} {d}_{i , j}} \qquad ( 4 )$$

The term “transcranial magnetic stimulation” or “TMS” as used herein refers to a non-invasive brain stimulation method which employs a magnetic field generator applied near the head to locally stimulate an electrical current within the brain. In embodiments, TMS includes repetitive transcranial magnetic stimulation or rTMS. Treatment with rTMS is comprised of multiple sessions (either daily across days or multiple times per day and across days) wherein TMS is delivered repetitively in a pattern that is intended to induce plasticity (defined as a change in brain activity). This plasticity could increase or decrease the activity of the brain region that is targeted. In embodiments, the rTMS is a “high frequency” protocol, involving stimulation at >5 Hz. In embodiments the rTMS is a “high frequency” protocol, involving stimulation at ≤1 Hz. In embodiments the rTMS is a “theta burst” protocol, involving stimulation with either a continuous or intermittent theta burst pattern. In embodiments, the rTMS provides a protocol involving stimulations at any value from or at about 1 Hz to about 5 Hz. In embodiments, the rTMS provides a protocol involving stimulations having more than one frequency.

TMS is a non-invasive technique that typically involves placing a coil near the patient's head to depolarize or hyperpolarize neurons of the brain. In particular, TMS uses electromagnetic induction to induce neuronal electrical currents using a rapidly changing magnetic field. A changing magnetic field leads to changing electrical currents by causing transient shifts in ions across neuron cell membranes. The brain region underneath the TMS coil is the primary target for the TMS effect, with further distant areas of the brain being impacted through the initial impulse delivered to the targeted region under the coil. TMS techniques typically act on a volume of brain tissue that is approximately two to three centimeters in diameter. TMS methods can include repetitive TMS (rTMS), single pulse TMS (spTMS), or paired pulse TMS (ppTMS).

In an example treatment protocol, daily rTMS induces long-lasting cortical neuromodulatory effects across broadly distributed regions. These effects are temporally and spatially removed from the onset and location of stimulation, but are highly predictive of clinical outcome. Mechanistically, non-invasive and invasive studies suggest that rTMS induces a reduction in early, local evoked gamma power and an early excitatory electrophysiological response, and an increase in later alpha power and slower inhibitory electrophysiological responses, suggesting a lasting alteration in the excitability of brain networks and altered interaction between brain regions and networks.

Treatment protocols for each type of TMS vary in duration, time course, pulse sequence, magnitude of stimulation and area of stimulation. Course of treatment can vary in duration from about one day, two days, three days, four days, five days, six days, seven days, one week, two weeks, three weeks, four weeks, five weeks, six weeks, seven weeks, eight weeks, or more. Frequency of TMS stimulation can vary (e.g., about 10, 20, or 30 Hz). TMS stimulation can be 1 Hz TMS, 3 Hz TMS, 5 Hz TMS, 7 Hz TMS, 10 Hz TMS, 15 Hz TMS, 20 Hz TMS, 25 Hz TMS, 30 Hz TMS or intermittent theta burst TMS. Paired pulse TMS can be administered at a time offset of about 10 milliseconds (msecs or ms), 20 msecs, 30 msecs, 40 msecs, 50 msecs, 100 msecs, 150 msecs, 200 msecs, 250 msecs, 300 msecs, or more. In embodiments, TMS can be administered to the right or left prefrontal cortices (e.g., left dorsolateral prefrontal cortex (DLPFC), right DLPFC, dorsal cingulate, dorsomedial prefrontal cortex, frontopolar cortex, ventrolateral prefrontal cortex.)

The term “right ECN post-traumatic stress disorder” or “right ECN PTSD” is meant to describe a subset of PTSD. Particularly, right ECN PTSD patient exhibits excessive right ECN rebound activity inhibition (e.g. upon stimulation of the right ECN, patients having right ECN PTSD will display a measurable suppression of rebound activity.) In embodiments, right ECN rebound activity inhibition may be elicited by TMS. TMS stimulation may be performed as single pulses, as paired pulses, triple pulses or quadruple pulses. Each of these protocols assesses the reactivity of the cortex being targeted and the amount of excitation or inhibition elicited by TMS stimulation. In embodiments, right ECN rebound activity inhibition may be measured by EEG. In embodiments, right ECN rebound activity inhibition may be measured as an average activity across the brain. In embodiments, right ECN rebound activity inhibition may be concentrated into particular brain regions (e.g. the right ECN, the frontal or parietal lobes, or along the midline of the brain). In embodiments, right ECN rebound activity inhibition may be measured at about 30-250 ms, about 50-150, about 60-120, about 80-110 or at about 110 ms following stimulation. In embodiments, right ECN rebound activity inhibition may be identified by an increased amplitude of the negative potential at about 100 ms (n100) following stimulation.

The term “biomarker” as used herein applies to a measure of a patient's biological functioning. In the present context a biomarker may be a pattern of neural functioning (e.g. a neural network connectivity or efficiency), an evoked response (e.g. a potential elicited by non-invasive brain stimulation), a pattern of behavioral or cognitive functioning (e.g. a performance on a memory deficit test), or a genetic or molecular marker or a combination of thereof.

The term “reduce” or “increase” is meant to alter negatively or positively, respectively, by at least 5%. An alteration may be by 5%, 10%, 25%, 30%, 50%, 75%, or even by 100%.

A “subject” as used herein refers to an organism. In certain embodiments, the organism is an animal. In certain embodiments, the subject is a living organism. In certain embodiments, the subject is a cadaver organism. In certain preferred embodiments, the subject is a mammal, including, but not limited to, a human or non-human mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of various embodiments, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### Post-Traumatic Stress Disorder (PTSD)

Posttraumatic stress disorder (PTSD) is a severe anxiety central nervous system disorder that may develop in response to exposure to an event resulting in psychological trauma. PTSD may be less frequent and more enduring than the more commonly seen posttraumatic stress. PTSD is believed to be triggered by a subject witnessing or experiencing any of a wide range of events that produce intense negative feelings of fear, helplessness, or horror. This experienced fear may trigger many split-second changes in the body to prepare to defend against or avoid the danger.

The “fight-or-flight” response is a healthy reaction meant to protect a person from harm. But it is believed that with PTSD, this reaction is altered. People suffering from PTSD may feel stressed or frightened even when they are not in danger. PTSD symptoms may include reliving the traumatic event in the form of flashbacks, or nightmares, for example. Further, symptoms of PTSD may include avoidance of places or things that are reminders of the experience; feeling emotionally numb; feeling anxious; and/or losing interest in formerly enjoyable activities. People suffering from PTSD may also experience hyperarousal symptoms such as being easily startled; feeling tense; and having difficulty sleeping for example.

Individuals impacted with PTSD require swift diagnosis and medical intervention. Usually, PTSD is diagnosed by psychiatrically trained professionals using questionnaires. Further diagnostic methods include assessment of neural activity. Methods of evaluating neural activity useful in diagnosis and monitoring of PTSD include, for example, functional magnetic resonance imaging (fMRI), magnetoencephalography (MEG) and monitoring of evoked responses (e.g. utilizing EEG to measure a response evoked by transcranial magnetic stimulation (TMS)). fMRI is a functional neuroimaging procedure using MRI technology that measures brain activity by detecting associated changes in blood flow. The technique relies on the fact that cerebral blood flow and neuronal activation are coupled. When an area of the brain is in use, blood flow to that region also increases. MEG is a functional neuroimaging technique for mapping brain activity by recording magnetic fields produced by electrical currents occurring naturally in the brain, using very sensitive magnetometers. Example methods of assaying neural activity include blood flow analysis (e.g. fMRI, or near infrared spectroscopy (NIRS)), functional connectivity analysis (e.g. electroencephalogram (EEG) or magnetoencephalography (MEG)), or structural connectivity analysis (e.g. diffusion-weighted structural connectivity analysis). Transcranial magnetic stimulation (TMS) is a non-invasive brain stimulation method which employs a magnetic field generator applied near the head to locally stimulate an electrical current within the brain. TMS can be used to evoke a response within the brain which can be monitored by methodologies described above, including EEG. Mapping an evoked response provides an additional methodology to assess neural connectivity.

Current PTSD diagnostic methods include Clinician-Administered PTSD Scale for DSM-5 (CAPS-5). CAPS-5, which is currently considered a “gold standard,” is a 30-item structured interview that corresponds to the Diagnostic and Statistical Manual of Mental Disorders, 5th Edition, (DSM-V) criteria for PTSD. CAPS-5 was designed to be administered by clinicians and clinical researchers who have a working knowledge of PTSD, but can also be administered by properly trained paraprofessionals. In embodiments, the PTSD diagnostic method may include any procedure or protocol that is in compliance with the Diagnostic and Statistical Manual of Mental Disorders, 4th Edition, (DSM-IV) criteria for PTSD and/or 10th revision of the International Statistical Classification of Diseases and Related Health Problems (ICD), a medical classification list by the World Health Organization (WHO) (ICD 10 or previous editions thereof).

Behavioral tests are further used for assessing PTSD. These tests can be administered by a professional, or completed electronically (e.g. on a computer interface). Example behavioral tests include card sorting analysis, reward or punishment learning tests, planning test, or navigation test. Behavioral assays comprise computerized or paper-and-pencil or tester-administered tests that require the individual being tested to perform particular tasks. Examples of outcomes of task performance is accuracy, response time or choice made. Behavioral assays have defined constructs that they intend to measure and an objective means to assess performance and test outcome.

Some symptoms of PTSD may be shared with other neurological disorders, for example traumatic brain injury (TBI). Damage to the brain by a physical force is broadly termed TBI. The resulting effect of TBI causes alteration of normal brain processes attributable to changes in brain structure and/or function.

### Methods of Monitoring Neural Activity

Dynamic monitoring of brain functioning can be useful in the assessment of PTSD. Examples of methods of assaying neural activity include blood flow analysis (e.g. fMRI, or near infrared spectroscopy (NIRS)), functional connectivity analysis (e.g. electroencephalogram (EEG) or magnetoencephalography (MEG)), or structural connectivity analysis (e.g. diffusion-weighted structural connectivity analysis).

Methods of that monitoring neural activity via blood flow changes can detect changes on the time scale of seconds, e.g., about 0.5 seconds, about 1 second, about 2 second, about 3 seconds, about 4 seconds, about 5 seconds, about 6 seconds, etc. Methods of monitoring neural activity via electric signal monitoring activity changes on the time scale of milliseconds, e.g. about 5 milliseconds, about 10 milliseconds, about 20 milliseconds, about 30 milliseconds, about 40 milliseconds, about 50 milliseconds, about 100 milliseconds, about 200 milliseconds, about 300 milliseconds, about 400 milliseconds, about 500 milliseconds, about 600 milliseconds, about 700 milliseconds, about 800 milliseconds, about 900 milliseconds.

Methods that monitor electrical activity (e.g. EEG) can detect output locally, for example, in an area immediately adjacent and electrode, or across the brain, for example, as an average activity across an electrode array. EEG can be used to monitor evoked brain activity (e.g. an evoked response elicited by TMS). In embodiments, EEG can be used to monitor activity at about 30-250 ms, about 50-150, about 60-120, about 80-110 or at about 110 ms following stimulation.

### Neural Connectivity

Connectivity efficiency refers to the degree of connectivity between two or more cognitive regions (e.g. 10 or more, 20 or more, or 30 or more cognitive regions). In one example, connectivity efficiency may be a measure of functional and/or anatomical connectivity. In another example, connectivity efficiency may approximate direct anatomical connectivity via synaptic connection. In another example, connectivity may be determined by monitoring a brain's response to non-invasive stimulation. These different types of connectivity efficiency may be, but are not necessarily, interrelated. For example, closely correlated activity amongst two brain nodes may not be indicative of a direct anatomical connection. Measures of functional connectivity can be assessed using fMRI, MEG, EEG or fNIRS. For example, fMRI functional connectivity can be determined between the time courses of two or more brain regions in an fMRI scan by correlating these time courses. MEG or EEG functional connectivity can be done through methods such as correlations between the amplitude of power envelopes of two regions in an MEG or EEG test, assessment of their coherency or phase locking. All of these methods relate the amplitude or timing of regional signals to each other and thus index communication of information between these regions.

Connectivity efficiency can be quantified by applying graph theory to measures of neural activity or connectivity. Mathematical models derived from graph theory allow for calculation of metrics use to quantify connectivity efficiency including “global efficiency”, “system segregation”, “participation coefficient” and “closeness centrality”.

Brain structure and function can be assessed on many levels, from analysis of gene expression in an anatomical area to the physical structure or topology of a brain region, to the synchronous or phasic firing of disparate cognitive nodes. Brain structures may be designated by either their anatomical area, or by grouped by neural functioning.

Cognitive regions or nodes are continuous physical portions of the brain (e.g. cerebral cortex, hippocampus, thalamus or cerebellum) that supports a cognitive process. Cognitive regions may include, for example a gyrus, a sulcus or an area covering a collection of gyri or sulci. Cognitive regions may be grouped by associated function, activity, or connectivity into cognitive networks (also called cognitive modules). Cognitive networks or modules are groupings of cognitive regions or nodes that support associated cognitive processes. Example cognitive networks include the frontoparietal network (also called the executive control, central executive, or attentional network), the dorsal attention network (also called the visuospatial or spatial attention network), the salience network (also called the ventral attention or cingulo-opercular network) and the default mode network.

Each cognitive network may comprise a number of cognitive nodes or cognitive regions, identifiable by, for example, independent component analysis (ICA). Other methods include principle components analysis (PCA), graph community detection algorithms, partial correlations and other methods.

### Diagnosis and Treatment of PTSD

Systems and methods provided herein may be utilized in the diagnosis and treatment of PTSD. Using known methods of assessing neural functioning described hereinabove, connectivity between cognitive regions of a subject may be assessed. Connectivity may furthermore be assessed between cognitive modules. In an example, fMRI is used to assess blood flow to the brain. fMRI output may be assessed, for example by using Cohen's D as a measure of effect size, to develop mathematical models of connectivity between cognitive regions. In some embodiments, neural activity assays measure blood flow. In some embodiment, neural activity assays measure electrical stimulus. In some embodiments, a Minimum Spanning Tree (MST) is used to evaluate connectivity. Connectivity can further be assessed by several different numerical outputs calculated utilizing an MST (e.g., global efficiency, system segregation, participation coefficient, and closeness centrality.

Connectivity may further be assessed by monitoring a brain response to external stimulation. For example, a response to TMS may be monitored by EEG. In embodiments, TMS is repetitive TMS (rTMS) or single TMS pulses. In embodiments, TMS is applied locally (e.g. to nodes within the right or left executive control network (ECN), or right or left salience network (SN)). In embodiments, a response to local non-invasive brain stimulation may be monitored by an EEG array. In embodiments, an evoked response may be monitored locally in a particular brain region or lobe. In embodiments, an evoked response may be monitored as an average response across the brain. In embodiments, an evoked response is monitored at about 10-300 ms, 20-250 ms, 20-150 ms, 20-130 ms, 30-100 ms, 30 ms, 60 ms, 100 ms, or 250 ms after stimulation. In embodiments, a subset of patients exhibits a response which acts as an excessive rebound inhibition (e.g. the brain putting the “brakes” on runaway excitation that may lead to a seizure or other abnormal pattern of brain activity). In embodiments, excessive rebound activity inhibition occurs at about 70-130 ms, about 80-120 ms, about 90-110 ms, or at about 100 ms after stimulation. In embodiments, excessive rebound activity inhibition occurs upon stimulation of the right ECN. In embodiments, excessive rebound activity inhibition is measured as an average activity across the brain.

Using the above metrics for connectivity a comparison can be made between the connectivity of an assessed subject and the average connectivity of a healthy control set. In some embodiments, a control set will be calibrated by demographic (e.g., age, gender, occupation, physical characteristics), by an individual scanner used (e.g., a single fMRI machine), or by institution (e.g. a particular hospital). In some embodiments, comparisons may be made against a single, universal control set.

In some embodiments, subjects with PTSD had lower average network efficiency and network segregation. In some embodiments, subjects with PTSD have a greater reduction in between module connectivity than within module connectivity. In some embodiments, PTSD patients do not display the inverse correlation between integration and segregation normally seen in healthy controls.

In some embodiments, behavioral tests may be administered to a subject suspected of, or at risk of having PTSD. In some embodiments, behavioral tests are administered in conjunction with objective measures of neural activity. In some embodiments, behavioral assays may be a standalone assessment.

In some embodiments, a behavioral assay may assess a complex cognitive behavioral deficiency (e.g., a memory deficiency). In some embodiments, memory deficiency may be a long term memory deficiency, a working memory deficiency, a short term memory deficiency, a delayed recall deficiency or an immediate recall deficiency.

In some embodiments, subjects with PTSD have impaired delayed recall paired with decreased cognitive network efficiency. In some embodiments, effects on memory are not paired with effects on system segregation or average network connectivity strength.

In some embodiments, both a behavioral assessment and neural activity assessment will be performed. In some embodiments, the combined assays can distinguish PSTD from TBI. For example, subjects with TBI, but not PTSD may be memory deficient, without any difference in cognitive network efficiency from a control group.

Mathematical modeling of neural activity and behavioral evaluation may be used alone or in concert to evaluate subjects for PTSD. In some embodiments, in patients diagnosed with PTSD, mathematical modeling of neural activity may further be used to predict treatment outcome. In some embodiments, subjects with PTSD who exhibit impairments in network efficiency and/or memory do not benefit from exposure therapy.

The main treatments for people with PTSD include psychotherapy, medications, non-invasive stimulation or a combination thereof. Many different types of therapy have been utilized in the treatment of PTSD, including, for example, prolonged exposure therapy, cognitive processing therapy, cognitive behavioral therapy, eye movement and desensitization therapy, acceptance and commitment therapy, and interpersonal psychotherapy. Types of non-invasive stimulation include TMS, including rTMS.

Treatment with rTMS is comprised of multiple sessions (either daily across days or multiple times per day and across days) wherein TMS is delivered repetitively in a pattern that is intended to induce plasticity (defined as a change in brain activity). This plasticity could increase or decrease the activity of the brain region that is targeted. In embodiments the rTMS would be a “high frequency” protocol, involving stimulation at >5 Hz. In embodiments the rTMS would be a “high frequency” protocol, involving stimulation at <=1 Hz. In embodiments the rTMS would be a “theta burst” protocol, involving stimulation with either a continuous or intermittent theta burst pattern. Theta burst refers to a pattern of stimulation generally having brief bursts at very high frequency (e.g. three pulses at 50 Hz) arranged in a lower frequency pattern (e.g. each burst being delivered at 5 Hz, which corresponds to the theta frequency range [4-8 Hz]). In embodiments, a continuous pattern of theta burst rTMS refers to stimulation with a patterns such as that above without pause. In embodiments, an intermittent theta burst pattern of rTMS refers to periodic stimulation with patterns such as the above pattern (e.g. theta burst stimulation for two seconds, followed by an eight second pause, and then resuming as a repeating cycle).

Antidepressant medications are the most commonly used medication in the treatment of PSTD. Sertraline (Zoloft®) and paroxetine (Paxil®), both of which are antidepressants, have been approved by the FDA for treating people with PTSD and are administered systemically, typically orally. Other example antidepressant medications include Abilify® (ariprazole), Adapin® (doxepin), Anafranil® (clomipramine), Aplenzin® (bupropion), Asendin® (amoxapine), Aventyl HCI® (nortriptyline), Brintellix® (vortioxetine), Celexa® (citalopram), Cymbalta® (duloxetine), Desyrel® (trazodone), Effexor XR® (venlafaxine), Emsam® (selegiline), Etrafon® (perphenazine and amitriptyline), Elavil® (amitriptyline), Endep® (amitriptyline), Fetzima® (levomilnacipran), Khedezla® (desvenlafaxine), Latuda® (lurasidone), Lamictal® (lamotrigine), Lexapro® (escitalopram), Limbitrol® (amitriptyline and chlordiazepoxide), Marplan® (isocarboxazid), Nardil® (phenelzine), Norpramin® (desipramine), Oleptro® (trazodone), Pamelor® (nortriptyline), Parnate® (tranylcypromine), Paxil® (paroxetine), Pexeva® (paroxetine), Prozac® (fluoxetine), Pristiq® (desvenlafaxine), Remeron® (mirtazapine), Sarafem® (fluoxetine), Seroquel XR® (quetiapine), Serzone® (nefazodone), Sinequan® (doxepin), Surmontil® (trimipramine), Symbyax® (fluoxetine and the atypical antipsychotic drug olanzapine), Tofranil® (imipramine), Triavil® (perphenazine and amitriptyline), Viibryd® (vilazodone), Vivactil® (protriptyline), Wellbutrin® (bupropion), Zoloft® (sertraline), and Zyprexa® (olanzapine)

The most common side effects of antidepressants like sertraline and paroxetine, also administered systemically, include: headache, nausea, agitation, sexual problems, and/or sleeplessness or drowsiness. Other types of systemically-administered medications may also be prescribed for people suffering from PTSD, such as benzodiazepines, antipsychotics, or other antidepressants. There is little information on how well these medications work for people with PTSD.

Provided herein relates to systems and methods for the treatment of PTSD. The systems and methods allow for the tailoring of PTSD treatment for maximizing successful outcomes. A treatment algorithm is provided herein wherein a patient may be evaluated and assessed for a presence or absence of a biomarker. A biomarker may be a pattern of neural functioning (e.g. a neural network connectivity or efficiency), an evoked response (e.g. a potential elicited by non-invasive brain stimulation), a pattern of behavioral or cognitive functioning (e.g. a performance on a memory deficit test), or a genetic or molecular marker or a combination of thereof. A biomarker may be used to classify a subset of a disease population. For example, right ECN PTSD patients are a subset of PTSD patients exhibiting excessive right ECN rebound activity inhibition (e.g. upon stimulation of the right ECN, patients having right ECN PTSD will display a measurable suppression of rebound activity.) In embodiments, patients with right ECN PTSD have impaired memory. A treatment protocol is guided by a presence or absence of a biomarker and the treatment algorithm provided herein provides a mechanism to identify patient subpopulations for which a certain therapy would be most beneficial.

In embodiments, patient evaluation for a biomarker indicative of a PTSD patient subpopulation is multi-part. In embodiments, patients positive for a biomarker for the subpopulation are predicted to have poor outcomes with psychotherapy alone. In embodiments, patients are first screened for memory deficits. In embodiments, patients without memory deficits are candidates for psychotherapy. In embodiments, identification of memory deficits is followed by an evaluation of neural connectivity (e.g. by an imaging test, or by monitoring of an evoked response). In embodiments, where neural connectivity is assessed by monitoring of an evoked response, non-invasive brain stimulation may be applied to the right dorsolateral prefrontal cortex. In embodiments, patients are positive for a biomarker in which alternate PTSD therapies should be used alone, or in combination with psychotherapy, an evoked response exhibits excessive rebound inhibition. Excessive rebound inhibition is a neural response in which the “brakes” on runaway excitation that may lead to a seizure or other deranged pattern of brain activity elicited by the brain in response to non-invasive brain stimulation is excessive or outside a normal response. Those patients eliciting this excessive rebound inhibition activity may be deemed to have right ECN post-traumatic stress disorder. In embodiments, right ECN PTSD is treatable with TMS. In embodiments, TMS is rTMS administered to the right dorsolateral prefrontal cortex.

### Genetic Markers of PTSD

Disclosed herein also provides genetic markers for PTSD. A subject suspected of having or diagnosed with PTSD may be evaluated for particular genetic variations in addition to, or in the absence of the above described neural activity or behavioral assays. Genetic markers may suggest a predisposition to PTSD, or may be used in tailoring of patient specific treatment strategies. In some embodiments, genetic markers for predisposition to PTSD may be evaluated by comparing gene expression data from microarray data from specified brain regions to the numerical metrics of connectivity described above. In some embodiments, correlation between gene expression and cognitive region closeness centrality may identify a genetic marker for PTSD predisposition.

Genetic markers identified herein having inversely correlated gene expression and closeness centrality are CRHR1 (a receptor for the corticotrophin releasing hormone) and STMN1 (the microtubule regulating protein stathmin). In some embodiments, SNPs within a designated PTSD genetic marker may be correlated with PTSD predisposition. Example SNPs include rs110402 (SEQ ID NO: 2) and rs242924 (SEQ ID NO: 3) in CRHR1 (SEQ ID NO: 1), and rs182455 (SEQ ID NO: 5) in STMN1 (SEQ ID NO: 4).

### EXAMPLES

To ensure robustness and generalizability of results provided herein, it was examined whether these brain-behavior-diagnosis relationships replicated in a second, independent sample, to which directly applied cutoffs were determined from the first patient/control sample. Importantly, these two samples differed in diagnostic criteria, gender predominance, civilian/veteran composition, and site and data acquisition method. The relationship of network function and cognition to treatment outcome in PTSD with psychotherapy, the best-validated treatment available for this disorder²¹, were then examined. Finally, diagnostic specificity relative to traumatic brain injury (TBI), another common consequence of trauma is disclosed herein.

### Example 1: Methods

Participants

Study 1 included 95 unmedicated right-handed subjects in the primary component of this study, including 59 patients with PTSD, and 36 trauma-exposed healthy subjects (demographics and clinical characteristics in Table 1). All participants were recruited and scanned at Stanford University after signing an institutional review board-approved informed consent. Psychiatric diagnoses, or absence thereof for controls, were based on DSM-IV criteria using the Clinician-Administered PTSD Scale (CAPS)⁴⁹ for PTSD and the Structured Clinical Interview for the Diagnostic and Statistical Manual of Mental Disorders Axis I (SCID I) for other Axis I disorders⁵⁰. Intelligence quotient (IQ) was estimated using the Wechsler Abbreviated Scale of Intelligence (WASI)⁵¹. Participants were permitted to meet diagnostic criteria for comorbid mood and anxiety disorders secondary to PTSD. General exclusion criteria for both groups included the following: a history of psychotic, bipolar or substance dependence (within 3 months for patients and lifetime for controls), a history of a neurological disorder, greater than mild traumatic brain injury (i.e. >30 minutes loss of consciousness or >24 hour post-trauma amnesia), claustrophobia, and regular use of benzodiazepines, opiates, thyroid medications, or other CNS medication. Trauma-exposed healthy controls were required to have experienced a criterion A trauma, but not meet lifetime criteria for any Axis 1 psychiatric disorder, including PTSD. The groups did not differ with respect to age, education or gender (p's>0.35). Of these PTSD patients, 51 participated in the treatment study described below, along with 15 additional patients who were on stable doses of an antidepressant (demographic and clinical characteristics in Table 5).

TABLE 1
Demographic and clinical characteristics of participants with means and (standard
deviations) for the analyses of behavior, network topology and their relationships.

| | Study 1 brain/behavior | | | | |
|---|---|---|---|---|---|
| | sample | | Study 2 replication sample | | |
| | healthy | PTSD | Healthy | PTSD | |
| | (N = 36) | (N = 59) | (N = 23) | (N = 46) | TBI (N = 78) |
| Age (yr) | 34.6 (11.7) | 36.9 (11.1) | 30.7 (8.5) | 32.8 (7.3) | 32.7 (6.9) |
| Education (yr) | 15.3 (2.4) | 15.0 (2.5) | 14.4 (3.3) | 14.2 (3.4) | 15.0 (2.4) |
| Gender (% female) | 53% | 63% | 9% | 9% | 4% |
| WASI intelligence | 112.3 (13.1) | 111.3 (10.7) | 108.7 (10.7) | 102.5 (11.6) | 106.7 (10.7) |
| quotient (IQ) | | | | | |

self-report

| | | | | | |
|---|---|---|---|---|---|
| Beck Depression | 1.6 (1.9) | 22.6 (8.9) | 2.4 (3.7) | 18.9 (11.5) | 11.7 (10.5) |
| Inventory | | | | | |
| WHOQOL physical | 87.6 (11.4) | 54.1 (18.6) | 84.2 (14.1) | 60.0 (17.6) | 69.1 (17.6) |
| health | | | | | |
| WHOQOL | 75.5 (11.6) | 40.4 (15.3) | 79.0 (16.1) | 52.8 (19.0) | 60.2 (18.9) |
| psychological health | | | | | |
| WHOQOL social | 77.1 (19.8) | 35.9 (22.9) | 70.2 (18.1) | 46.2 (25.4) | 56.4 (22.1) |
| relationships | | | | | |
| WHOQOL | 79.4 (14.8) | 54.1 (20.7) | 72.9 (14.6) | 57.6 (17.0) | 63.5 (17.1) |
| environment | | | | | |

DSM-IV clinician scales

| | | |
|---|---|---|
| CAPS total | 2.4 (3.4) | 68.5 (16.1) |
| CAPS re- | .7 (1.5) | 17.9 (6.2) |
| experiencing | | |
| CAPS avoidance | .7 (1.6) | 28.5 (8.5) |
| CAPS hyperarousal | 1.0 (1.8) | 22.1 (5.7) |

DSM-5 clinician scales

| | | | |
|---|---|---|---|
| CAPS total | 2.9 (2.8) | 31.4 (10.6) | 9.0 (6.8) |
| CAPS re- | .8 (1.3) | 7.4 (3.6) | 1.9 (2.4) |
| experiencing | | | |
| CAPS avoidance | .18 (39) | 3.9 (1.5) | .65 (1.2) |
| CAPS hyperarousal | .24 (.56) | 10.5 (5.2) | 1.8 (2.8) |
| CAPS negative | 1.7 (1.6) | 9.6 (3.1) | 4.6 (3.7) |
| cognitions | | | |

TABLE 5
Biomarker characteristics of verbal memory and efficiency separately.

| Verbal | group | | |
|---|---|---|---|
| Memory | healthy | PTSD | TBI |
| Intact | 19 (90%) | 30 (65%) | 64 (83%) |
| memory | | | |
| Impaired | 2 (10%) | 16 (35%) | 13 (17%) |
| memory | | | |

PTSD versus healthy χ² = 4.68, p = .03
PTSD versus TBI χ² = 5.12, p = .024

| | group | | |
|---|---|---|---|
| Efficiency | healthy | PTSD | TBI |
| Good | 9 (41%) | 10 (22%) | 37 (47%) |
| efficiency | | | |
| Poor | 13 (59%) | 36 (78%) | 41 (53%) |
| efficiency | | | |

PTSD versus healthy χ² = 2.71, p = .1
PTSD versus TBI χ² = 8.12, p = .004

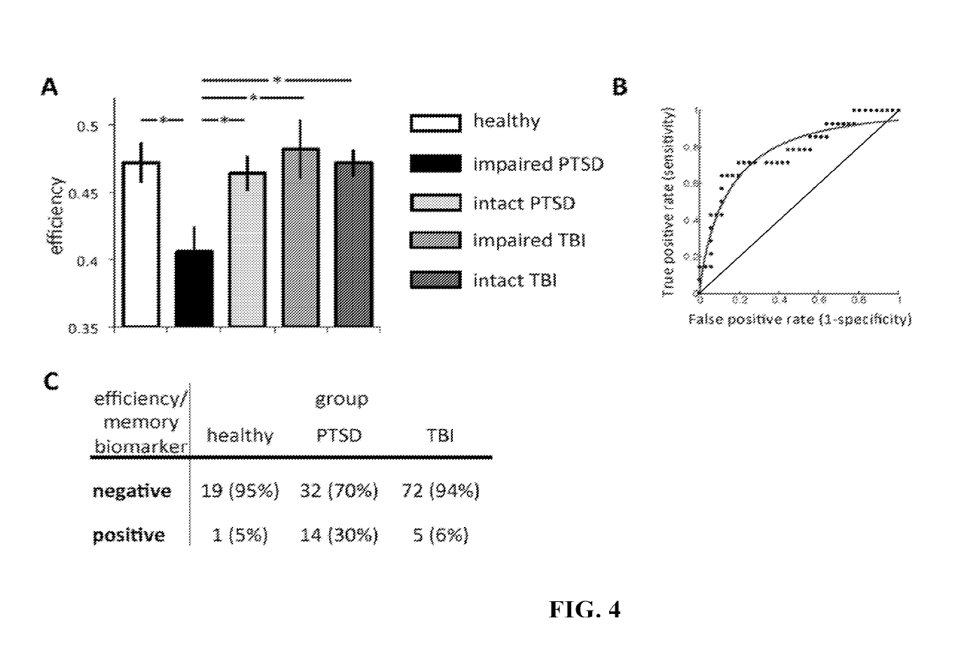

Study 2 involved 147 participants, including 46 patients with PTSD, 23 trauma-exposed healthy participants, and 78 participants with traumatic brain injury (TBI) and no PTSD (see Table 1). Participants were recruited and scanned at either Stanford University or New York University after signing an institutional review board-approved informed consent. All participants were combat veterans serving during the Operation Iraqi Freedom (Iraq), Operation Enduring Freedom (Afghanistan) and Operation New Dawn periods. Similar inclusion/exclusion and diagnostic criteria were used as above except that diagnoses were based on DSM-5⁵² criteria rather than DSM-IV⁵³. TBI was diagnoses based on loss of consciousness⁵⁴. Those participants with both PTSD and TBI were assigned to the PTSD group. A regular stable dose of psychotropic medication (primarily antidepressants) was used by 28% of the PTSD participants and 9% of the TBI participants. Medication use, however, did not confound the results (FIG. 4).

The effects of CRHR1 and STMN1 genotypes were assessed in a group of 119 participants (72 females; mean age 35.7 years (SD 11.1); mean education 15.5 years (SD 3.0), including 38 healthy participants and 81 patients meeting criteria for primary PTSD or major depression (diagnoses were entered as covariates in the analyses).

Participants across all studies also reported on quality of life across physical, psychological, social relationship and environmental domains using the World Health Organization's WHOQOL-BREF survey ⁵⁵. Participants in Study 2 reported on alcohol use with the Alcohol Use Disorders Identification Test (AUDIT)⁵⁶.

Treatment Randomization (Study 1 Treatment Sample)

Following completion of baseline clinical assessments and fMRI scan, participants were randomized with a 50/50 probability to one of two arms: 1) Immediate treatment with prolonged exposure therapy; or 2) Treatment waitlist. A total of 66 individuals were randomized, with 36 being randomized to immediate treatment, and 30 to treatment waitlist. If randomized to immediate treatment, participants commenced treatment with a clinical psychologist trained to deliver prolonged exposure therapy. If randomized to treatment waitlist, individuals were instructed they would have a 10 week waiting period after which they would undergo a second clinical assessment and fMRI scanning session. After completion of this second assessment, individuals on treatment waitlist were then assigned to a study therapist for completion of prolonged exposure therapy.

Treatment Frequency and Length

Treatment sessions occurred on either a once or twice-weekly basis, for a total of either 9 or 12 90-minute sessions. At sessions 2, 4, 6, and 8 individuals were administered the PTSD-Checklist Civilian Version for DSM-IV⁵⁸ as well as the Beck Depression Inventory-II⁵⁹ to track response to treatment. The benchmark used to establish adequacy of treatment response at Session 9 and subsequent termination was at least a 70% reduction in Session 8 PCL-C scores from the PCL-C total score at intake. If individuals met this benchmark, they were given the option to discontinue treatment after Session 9. If individuals did not meet this benchmark and/or wished to continue for an additional 3 sessions, treatment was terminated after Session 12. If treatment continued to 12 sessions, PCL and BDI measures were also administered at Sessions 10 and 12.

Therapist Competency and Supervision in Prolonged Exposure

All psychologists received training in delivery of prolonged exposure and were deemed to meet competence in delivery of the treatment by one of the treatment developers, consultant to the study, and clinician supervisor Barbara Rothbaum, Ph.D. Dr. Rothbaum provided weekly group supervision to study therapists and reviewed video recordings of treatment sessions to rate compliance with the treatment protocol. Dr. Rothbaum watched the entirety of the first three treatment sessions for each patient to ensure therapist familiarity and competence with all major components of the treatment (all delivered in the first three sessions), and she continued to review relevant portions of remaining sessions as directed by study therapists. All study therapists demonstrated good compliance with the therapy protocol and with no significant deviations, as demonstrated by good-to-excellent supervisor ratings of treatment session adherence.

Treatment Structure

Prolonged exposure therapy was delivered according to manualized procedures⁶⁰. All sessions were audio recorded on a digital voice recorder (entrusted to the patient to take home with them and for use in completing imaginal exposure homework assignments) as well as a digital video recorder (for the purposes of assessing treatment adherence, therapist competency, and clinical supervision). In brief, the structure and progression of treatment is as follows. Session 1 consisted of psychoeducation on posttraumatic stress disorder symptoms, the rationale for treatment, and treatment structure. It also involved additional assessment by the therapist of trauma history (including the index trauma, already established at intake), current symptoms, and current impairment. Breathing retraining was taught at the end of Session 1 and practiced collaboratively in session, which consisted of a normal inhalation and a controlled and slow exhalation with internal repetition of a calming word or phrase (e.g., “Calm”) and a pause between exhalation and next inhalation.

Session 2 consisted of homework review, self-report measures, a discussion of common reactions to trauma, a rationale for exposure as a treatment tool, construction of an exposure hierarchy for in-vivo exposure exercises, and selection of 2 to 3 hierarchy items for homework practice. Session 3 involved homework review, a brief rationale for imaginal exposure, and conduction of the first imaginal exposure in session for 45-60 minutes. This was followed by a processing portion in which the therapist and participant discussed the participant's experience of the exposure, any insights received through that process, and areas to be further addressed in future exposures. Homework was then assigned (including completion of in-vivo exposures and imaginal exposures daily, and practice of breathing retraining). Session 4 consisted of the same format as Session 3.

Beginning in Session 5, the concept of trauma memory “hotspots” was discussed with participants, which were points in the memory during which the participant expressed the highest level of distress. The in-session imaginal exposure began to shift towards emphasizing hotspots in the memory in Session 5, at earliest, and sometimes Session 6 if agreed to be clinically appropriate by the participant and therapist. Session 6, 7, and 8 involved a similar format, with homework review, imaginal exposure, processing, and homework assignment. For participant's reaching the PCL clinical benchmark in Session 8, and agreeing to end in 9 sessions, Session 9 consisted of homework review, a brief imaginal exposure of the entire trauma memory conducted in-session (20-30 minutes), a brief processing, and a final review of treatment progress and skills acquired. For participants not reaching the clinical benchmark and/or wishing to continue for an additional 3 sessions, Sessions 9-11 maintained the same format as Sessions 4-8. In this case, Session 12 served as the final session (which assumed the aforementioned format).

Post-Treatment Clinical Assessment

Approximately 4 weeks following the final treatment session, participants were invited to return to complete a post-treatment clinical assessment. A 4-week period was chosen to intercede between final session and post-treatment assessment in order to allow treatment changes to consolidate and symptom levels to equilibrate. Participants were administered the CAPS and SCID again at post-treatment to assess change in PTSD symptoms and comorbid diagnoses.

Behavioral Tasks

All cognitive tasks were performed on a computer, as previously described ^{61,62}. Behavioral data were available on 86 of participants in Study 1, 144 participants in Study 2 and 110 participants in the genotyping analysis.

Word Learning Task (Verbal Memory).

Participants are presented visually with 20 English words per learning block, one at a time, which they are asked to memorize. Words are closely matched on concreteness, number of letters and frequency. Immediate recall is assessed by presentation of 20 sets of 3 words, one of which was previously presented (which participants are asked to indicate). There are three of these learning and immediate recall blocks, with word combinations altered across blocks. A delayed recall test is done 10 minutes later with another 20 sets of 3 words.

Continuous Performance Test.

To assess sustained attention, a series of letters (B, C, D, or G) is presented to the participant on the computer screen (for 200 msec or ms), separated by an interval of 2.6 sec. Participants are asked to indicate with a key press if the same letter appears twice in a row, which occurs on 12 of the 63 trials.

Choice Reaction Time Task.

Participants are required to attend to the computer screen, and indicate with a key press which of two black circles turned green on that trial. A total of 20 pseudo-randomly presented trials are displayed with a jittered inter-trial interval of 2-4 seconds.

MRI Data Acquisition and Preprocessing

General Scan Parameters.

All resting-state fMRI scans were eight minutes in length. Imaging for the healthy subjects and PTSD patients in Study 1 was performed on a 3 T General Electric 750 scanner at Stanford University. 29 axial slices (4-mm slice thickness) were acquired covering the whole brain, using a T2-weighted gradient-echo spiral-pulse sequence (repetition time, 2000 ms; echo time, 30 ms; flip angle, 80 degrees; slice spacing, 0.5; field of view, 22 cm; matrix size, 64×64)⁶³. An automated high-order shimming method based on spiral acquisitions was used before acquiring fMRI scans ⁶⁴ in order to reduce blurring and signal loss arising from field inhomogeneities. In addition, for retrospective correction of physiological motion effects in fMRI, RETROICOR was used ⁶⁵. For Study 2, the Stanford site acquired 32 axial slices with 3.5 mm thickness using an echo-planar gradient-echo T2-weighted pulse sequence (repetition time, 2000 ms; echo time, 29 ms; flip angle, 90 degrees; slice spacing, 0; field of view, 20 cm; matrix size, 64×64). The NYU site acquired data on a Siemens 3 T Skyra scanner using functional parameters matching those at Stanford above.

Preprocessing.

The first 5 acquired volumes (10 seconds) were dropped, the data were then motion corrected using FSL's mcFLIRT (http://fsl.fmrib.ox.ac.uk/fsl/fslwiki/MCFLIRT). The non-linear registration to standard space was performed using FSL's FNIRT, registration from functional to T1-weighted structural images was estimated using FSL's implementation of boundary-based registration (fsl.fmrib.ox.ac.uk/fsl/fslwiki/FLIRT_BBR). The mean white matter (WM) and cerebral spinal fluid (CSF) signal was estimated from the time-series using an MNI space defined WM/CSF mask transformed to the native functional space. The functional time-series was residualized with respect to the estimated WM/CSF signal. The data were then spatially smoothed with full-width half-maximum (FWHM) Gaussian of 6 mm. The data were residualized with respect to six motion parameters (estimated from mcFLIRT) to further account for motion effects. A bandpass filter was applied to data using cut-off frequencies of 0.008 Hz-0.1 Hz. Only subjects with maximum root mean square motion <3 mm were included in analyses.

fMRI Data Analysis

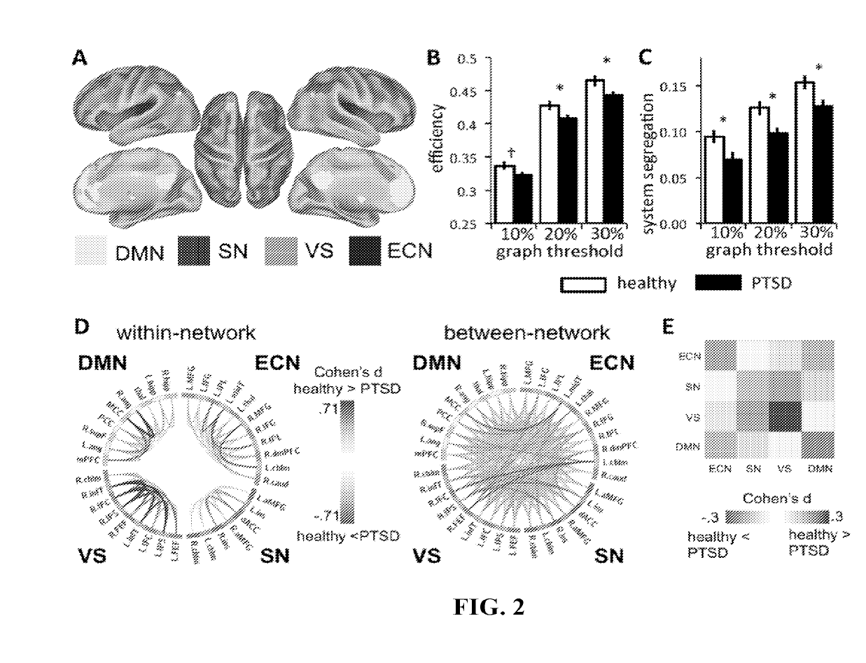

Regions of interest (ROIs) were defined based on a group independent component analysis (ICA) from an independent cohort ⁶⁶, wherein spatial maps derived from the ICA were used to define network communities or modules. The following modules were used for analysis based on their involvement in cognitive operations ¹⁸: the 1) default mode network (DMN), 2) salience network (SN), 3) visuospatial network (VS, also called the dorsal attention network), and 4) executive control network (ECN, also called the central executive or frontoparietal network). For each spatial map, each spatially disconnected region was defined as an ROI or node in the graph. Connectivity strength was estimated using Pearson's correlation coefficient between the mean signals from each pair of a priori ROIs, which were then converted to z-scores using Fisher's r-to-z transformation and converted to absolute values for graph analysis. Only those ROIs were included which had data from all participants. This resulted in 11 ECN nodes, 7 SN nodes, 9 VS nodes and 9 DMN nodes (FIG. 2A, Table 3).

TABLE 3
Region abbreviations

| Network | | |
|---|---|---|
| module | region name | region abbreviation |
| ECN | L middle frontal gyrus | L MFG |
| ECN | L inferior frontal gyrus | L IFG |
| ECN | L inferior parietal lobule | L IPL |
| ECN | L middle temporal gyrus | L midT |
| ECN | L thalamus | L thal |
| ECN | R middle frontal gyrus | R MFG |
| ECN | R inferior frontal gyrus | R IFG |
| ECN | R inferior parietal lobule | R IPL |
| ECN | R dorsomedial PFC | R dmPFC |
| ECN | L lateral cerebellum | L cblm |
| ECN | R caudate | R caud |
| SN | L anterior middle frontal gyrus | L aMFG |
| SN | L insula | L ins |
| SN | dorsal ACC | dACC |
| SN | R anterior middle frontal gyrus | R aMFG |
| SN | R insula | R ins |
| SN | L lateral cerebellum | L cblm |
| SN | R lateral cerebellum | R cblm |
| VS | L frontal eye fields | L FEF |
| VS | L intraparietal sulcus | L IPS |
| VS | L inferior frontal cortex | L IFC |
| VS | L inferior temporal gyrus | L infT |
| VS | R frontal eye fields | R FEF |
| VS | R intraparietal sulcus | R IPS |
| VS | R inferior frontal cortex | R IFC |
| VS | R inferior temporal gyrus | R infT |
| VS | R lateral cerebellum | R cblm |
| DMN | medial prefrontal cortex | mPFC |
| DMN | L angular gyrus | L ang |
| DMN | R superior frontal gyrus | R supF |
| DMN | posterior cingulate gyrus | PCC |
| DMN | mid-cingulate gyrus | MCC |
| DMN | R angular gyrus | R ang |
| DMN | thalamus | thal |
| DMN | L hippocampus | L hipp |
| DMN | R hippocampus | R hipp |

Graphs and Network Measures

Undirected graphs were constructed such that for each subject every node of the graph must be connected and that each graph contains an equal number of edges. A minimum spanning tree (MST) was first estimated to ensure that all nodes were connected. The MST is a tree connecting all nodes, such that the total edge weight is minimized. The motivation behind using the MST is that all regions of the brain should be connected in some way. From a mathematical standpoint, network metrics that rely on distance (e.g. path length) are ill-defined in the situation where a node of the graph is disconnected (infinite distance to all other nodes). For data provided herein, the MST guarantees that the path length from each node to every other node is finite. An important aspect of the thresholded graphs is that it set all edges larger than a threshold to zero, however the edge strengths are not binarized. Furthermore, since the metrics that are calculated are all weighted metrics, the addition of weak edges should have little impact on the network metrics.

Let αN be the N percentile node distance. Let MSTmin be the minimum node distance in the MST.

Two cases arise:
1. αN≥MSTmin: The MST is entirely encompassed within the percentile thresholded graph.
2. αN<MSTmin: The MST is not entirely contained within thresholded graph.
   In case 1, the use of the MST does not alter then threshold. In case 2, the M longest edges between nodes of the thresholded graph are replaced by the M edges of the MST that are not included in the graph. In other words, the subthreshold edges are swapped with the longest edges (smallest correlations) in the graph.

The MST was estimated using the Boost Graph Library's (BGL) implementation of the Kruskal's algorithm ⁶⁷. The strongest of the remaining edges were then added to the graph until the number of edges was equal to a specified percentage of the total number of possible edges. For each subject a graph was constructed setting the number of edges equal to 10%, 20%, and 30% of the total number of possible connections, as using different proportional thresholds ensures robustness of the findings to arbitrary methodological choices like graph thresholding^{68,69}. All graph metrics were calculated for each threshold and then averaged for the purpose of group comparisons since no interaction with graph threshold effect was found. The graph metrics examined were global efficiency, system segregation, participation coefficient, closeness centrality and degree. Weighted versions of each metric were used, as is described previously⁶⁹. Results were also examined in weighted unthresholded graphs.

Global efficiency is a single metric aimed at assessing overall connectivity by averaging efficiency over all network nodes. A node's efficiency is defined as the mean of the inverse-shortest-distance from that node to all other nodes. The global efficiency can be expressed as,

$${E}_{Global} = \frac{1}{n} {\sum }_{i ∈ N} \frac{{\sum }_{j ∈ N , i \neq j} \frac{1}{{d}_{ij}}}{n - 1 \\} , \qquad ( 1 )$$

where dᵢⱼ is the shortest distance between the i^{th} and j^{th} nodes, n is the number of nodes in the graph. Distances between two nodes are the inverse of their correlations, and the shortest distances were calculated using BGL's implementation of the Dijkstra's algorithm⁷⁰.

System segregation examines the community structure of a network, in particular, segregation was used to measure how well the networks follow an a priori community structure defined by ICA. System segregation is defined as the difference between the mean within-module and between-module connectivity relative to the mean within-module connectivity ⁴⁸. System segregation was calculated using,

$$S = \frac{{\overline{Z}}_{W} - {\overline{Z}}_{B}}{{\overline{Z}}_{W}} \qquad ( 2 )$$

where S is the system segregation measure, Z̅_{W} is the mean within-module connectivity and Z̅_{B} is the mean between-module connectivity. Z̅_{W} and Z̅_{B} are calculated by, respectively, averaging all the within-module and between module edge weights. The edge weights are Fisher's z-transformed Pearson's correlation estimates between each region.

The participation coefficient quantifies the degree to which a node serves as a communication hub between nodes outside its module and those within its module. The module definitions were implicit to the node definitions as they were derived from the ICA maps. Participation coefficient can be expressed as follows,

$${P}_{coeff} = 1 - {\sum }_{m ∈ M} {( \frac{{k}_{i} ( m )}{{k}_{i}} )}^{2} , \qquad ( 3 )$$

where M is the set of modules, kᵢ is the node degree and kᵢ(m) is the total connection strength from node i to module m.

Closeness centrality is defined for each node as inverse of the average shortest-distance. Closeness centrality, which is very tightly related to efficiency, is defined below.

$$CC = \frac{1}{L} = \frac{n - 1}{{\sum }_{j ∈ N , i \neq j} {d}_{i , j}} \qquad ( 4 )$$

Gene Expression Analyses

The AIBS has made public human microarray data on anatomically defined samples across multiple locations in the human brain, drawn from six post-mortem donors, two contributing both hemispheres and four contributing only the left hemisphere ⁷¹. Data was used only from left-sided samples in order to maximize potential overlap across donors, since the primary interest was differential gene expression across brain regions. Anatomical location was referenced to a post-mortem MRI scan, allowing determination of where the sample corresponded to in MNI coordinates. As the AIBS data set used in this study was the same as used in a prior study examining the relationship between a region's membership in large-scale functional connectivity networks and patterns of expression across the transcriptome ⁷², relevant methods are described briefly herein. As in this prior study, only cortical samples were used to avoid major transcriptional dissimilarities across brain regions due to different cellular makeup (e.g. cortex, basal ganglia, cerebellum etc.) and because the vast majority of regions within the cognition-related networks were cortical.

Microarray sample locations were aligned to the network regions by selecting those samples lying within the mask for that region or within a distance of one in-plane voxel of the outside edge of the mask, averaging samples if there were multiple matches for a region. The optimal microarray probe for each gene was determined as previously described ⁷², with the additional change that less variable probes (as measured by standard deviation divided by absolute value of median) are considered if the most variable probe does not satisfy other requirements (having an Entrez ID, and being called at least in one sample), in order to maximize inclusion of data from PTSD-related genes. This resulted in microarray data from 17,878 genes. This set of PTSD-related genes was identified as the set of available genes from those summarized in very a recent genome-wide association study (Table 4 in ⁷³).

Next, the closeness centrality of each network node (determined using data from healthy participants) was correlated against the expression level of each of the PTSD-related genes for all left-sided regions for which matching microarray data were found. As this was done separately for each donor, the combined correlation coefficient across donors was then calculated using Fisher's method and the combined p-value using Stouffer's method (yielding a one-sided p-value). After an FDR correction for multiple comparisons (one-sided q<0.025 threshold), two genes whose expression was significantly related to the closeness centrality of each network node were identified.

Genotyping Analyses

Two well-characterized SNPs in CRHR1 (SEQ ID NO: 1, GenBank Accession Number: NM_001145146.1) (SNPs: rs110402, SEQ ID NO: 2; and rs242924, SEQ ID NO: 3) and one in STMN1 (SEQ ID NO: 4, GenBank Accession Number: NM_001145454.2) (SNP: rs182455; SEQ ID NO: 5) were genotyped to determine whether polymorphisms in the genes identified in the gene expression analyses in turn are associated with altered network efficiency or delayed verbal memory recall. All of these genes have been shown to be associated with altered brain function ⁷⁴⁻⁷⁸, and hence are good candidate SNPs for determining the impact of CRHR1 and STMN1 on the brain and behavioral aspects of cognitive dysfunction. Since rs110402 and rs242924 are in high linkage disequilibrium at D′=0.99⁷⁹, and the genotyping reactions failed for several individuals at the rs242924 locus who had rs110402 genotypes, only the rs110402 SNP was analyzed to maximize sample size. Genotype frequencies and Hardy-Weinberg equilibrium p-values are in Table 10. Based on prior work, analyses were conducted by comparing G homozygotes to A carriers for rs110402 and T homozygotes to C carriers for rs182455.

TABLE 10
Hard-Weinberg equilibrium for the genotypes examined

| SNP | Hardy-Weinberg p-value | genotypes |
|---|---|---|
| rs110402 | .439 | GG: 28 (23.5%) |
| (CRHR1) | | AG: 52 (43.7%) |
| | | AA: 39 (32.8%) |
| rs182455 | .636 | CC: 41 (36.3%) |
| (STMN1) | | CT: 50 (44.2%) |
| | | TT: 22 (19.5%) |

>CRHR1, SEQ ID NO: 1, GenBank Accession Number: NM_001145146.1

| | |
|---|---|
| 1 | ggcgaggccg gcaagaggcg gccgcgggcc gggctgcgtc gggaaacggc ggccagactt |
| 61 | ccccgggaag gggcgagcga gagccgggcc gggccgggcc gggccgcggg gccgggaagc |
| 121 | gccgagccgg gcatctcctc accaggcagc gaccgaggag cccggccgcc caccccgtgc |
| 181 | cgcccgagcc cgcagccgcc cgccggtccc tctgggatgt ccgtaggacc cgggcattca |
| 241 | ggacggtagc cgagcgagcc cgaggatggg agggcacccg cagctccgtc tcgtcaaggc |
| 301 | ccttctcctt ctggggctga accccgtctc tgcctccctc caggaccagc actgcgagag |
| 361 | cctgtccctg gccagcaaca tctcaggact gcagtgcaac gcatccgtgg acctcattgg |
| 421 | cacctgctgg ccccgcagcc ctgcggggca gctagtggtt cggccctgcc ctgccttttt |
| 481 | ctatggtgtc cgctacaata ccacaaacaa tggctaccgg gagtgcctgg ccaatggcag |
| 541 | ctgggccgcc cgcgtgaatt actccgagtg ccaggagatc ctcaatgagg agaaaaaaag |
| 601 | caaggtgcac taccatgtcg cagtcatcat caactacctg ggccactgta tctccctggt |
| 661 | ggccctcctg gtggcctttg tcctctttct gcggctcagg ccaggctgca cccattgggg |
| 721 | tgaccaggca gatggagccc tggaggtggg ggctccatgg agtggtgccc catttcaggt |
| 781 | tcgaaggagc atccggtgcc tgcgaaacat catccactgg aacctcatct ccgccttcat |
| 841 | cctgcgcaac gccacctggt tcgtggtcca gctaaccatg agccccgagg tccaccagag |
| 901 | caacgtgggc tggtgcaggt tggtgacagc cgcctacaac tacttccatg tgaccaactt |
| 961 | cttctggatg ttcggcgagg gctgctacct gcacacagcc atcgtgctca cctactccac |
| 1021 | tgaccggctg cgcaaatgga tgttcatctg cattggctgg ggtgtgccct tccccatcat |
| 1081 | tgtggcctgg gccattggga agctgtacta cgacaatgag aagtgctggt ttggcaaaag |
| 1141 | gcctggggtg tacaccgact acatctacca gggccccatg atcctggtcc tgctgatcaa |
| 1201 | tttcatcttc cttttcaaca tcgtccgcat cctcatgacc aagctccggg catccaccac |
| 1261 | gtctgagacc attcagtaca ggaaggctgt gaaagccact ctggtgctgc tgcccctcct |
| 1321 | gggcatcacc tacatgctgt tcttcgtcaa tcccggggag gatgaggtct cccgggtcgt |
| 1381 | cttcatctac ttcaactcct tcctggaatc cttccagggc ttctttgtgt ctgtgttcta |
| 1441 | ctgtttcctc aatagtgagg tccgttctgc catccggaag aggtggcacc ggtggcagga |
| 1501 | caagcactcg atccgtgccc gagtggcccg tgccatgtcc atccccacct ccccaacccg |
| 1561 | tgtcagcttt cacagcatca agcagtccac agcagtctga gctggcaggt catggagcag |
| 1621 | cccccaaaga gctgtggctg gggggatgac ggccaggctc cctgaccacc ctgcctgtgg |
| 1681 | aggtgacctg ttaggtctca tgcccactcc cccaggagca gctggcactg acagcctggg |
| 1741 | ggggccgctc tccccctgca gccgtgcagg actctagctc atgagtggaa agtcacctac |
| 1801 | aggactgggc cgggcccagg gcctctggct tccctgccca atcctccctg gagaagggac |
| 1861 | atgggaatga attgaaatgg ggcgctggac acctacagca gcacgcatgt ccctccaagg |
| 1921 | ctgtcttctc ccagagcaca agaaggccag cccactgggc cctggggctg ccctcggcaa |
| 1981 | ccgtggggag gccatttgct gccctggggc atcatgggca actcgtgaca gcctctgact |
| 2041 | caccacgatg acgcctctgg acctcggtga tgccttccga caccactggg aaccaagggc |
| 2101 | cctcactcag gaaccctgga gacagaagtc aggtgtcatc atcagacttg cggccacagc |
| 2161 | actagagtca cccccccagg cctccagaac cttactggca ctgtggcact gccaccagca |
| 2221 | atgccctgcc ttgctgcctt caccctgaac atttagtacc ctgcaggcca ggccagcttc |
| 2281 | ccctcactta accaccccat accagtcacc tcctgctcct tttcctcttt tgtgagaaga |
| 2341 | tgggggctgg agggggcaga gtggcctgtg agcaagagcc aggggtgtcc cagtcccagc |
| 2401 | ctctggggca gagcttgtag ccctggatgg cctctggggc aggaccacta gctaagcaag |
| 2461 | ccaggagaag acccctgccc aagtggctct tgggacaacg tgctgcttac actccaggtg |
| 2521 | tggaccggcc gcagccccca ctgacctgcc catgtccaga gggactggac agccagggca |
| 2581 | gggctttggg gggcactaga agatgagggt gtcggctgtg aggcgggtgg ctggtataaa |
| 2641 | taatatttat cttttcaacc agcaaaaaaa aaaaaaaaaa a |

>rs110402, SEQ ID NO: 2
GGGGAGGGGG GCCTTCTCAT GTCTGCAACC CTCTGCCCAT GCAGTCTGTG CCACCCCCCA
CCCCTGGTCA CCTGGTCAAC TCTGCATTCT TCAGAGGCCA GTTCCAGGGC CCGTCTGCTT
AGTAGTACCT GCCCAACTCT CCAAAGCAAA GCAGCTCAGT TCTCCCTCCA ATGTACCCCT
TTTCCCAGTG CACTCTGTAC ACTCACTGGA CCCTGTGCCC GTGAGGCTCA CAAGTAGAGC
ACTTAGACCA GCCTAAGTGC ACGAAAACCA CCTCGGCACA AAGCACTTAG GACCCAAGAC
ACTAATTAGA TGGCAAATAT ACAGGCTATA AGTTCTTATT CTCGCATCTT AATGTCCTGG
CGTGCACAGA GGAGCAGAGC AAATCTTGAT TCGTCAGAAA ATGAAGCCAA ATTCCAGGCA
AAGGGCTAGT CTTCCCTTGG CTGCCTAGAA CCCTGACACT TGTGTCCCTC TCTTGAATGT
GATGGTTCAC ACAGGCATTT TCTAAACACA GAGGACTGGT GTTG
GTTATGCAAA GAAAAATGCT TCTTAAAATT CCCAAACCAA CCTTTCCTCT CTGGAAAGGT
CTGCTCTGTA GCAGAATCTG TGCCTGTGCC ACCTCTTGCT CTGTGGAAGG TTTGTGAGGG
CTTCTGCCTT ATTCCTGAAT CATACTTCCA TGAAAAGTCC AGAGATTTCC TGCATTTTTC
CTCCCACCAT CCGCTGATCT CCTCTTCACC ATCCGCTGAT CTCCTCTTCA AGCTCCACTG
TGGTTTTCTA TCTCCATCAA TAGGCAATTA CTGCACTGGC AGGAACAGGC CCCCAAAGTT
TCAAGGATGT TGTTTGTTTG CCCTTaaaat ggagaatttt tttgtttgtt t ttttttga
gacagtcttg ctctgtcgcc caggctagag tgcagtggtg cgatctccgc tcactccaac
ctctgtctcc tgggttcaag caattctcct gcctcagcct cccaagtagc tgggattaca
ggcctgcacc accatgccca gctaagtttt gtgttttcag tagagatgag gtttcaccct
gttggccagg ctggtctcaa actcctgact tcaagtgatc agcccgcctc ggcctcccaa
agtgctggga ttacaggagt gagccaccat gctgggctaa aacaacaaaa aacaaaacaa
acaaaaaaaa aaaCACGGAG AATTCTTAAC CTGGGGAGCG GGCAGTGTGA AGATTTGCAA
ATAAATGCAG TTGGTGTGTG TGATGAAAAC CATTTTCCCG GGGAGGGTTC TTGGCTTCCT
CCACTTCTCA CCAACAACTA GCACCCTGAA ACAGTGGGAA CTACAGTCGA AGCATATGGT
AAATGGGGTG AACTGAACAT GTCATCTTCT CTGAAATCAA ACCAAAGTGC TTTTGGCCTT
TCTCAGATGC CTCTTGCTCC TCCACTCCAT CACCAGTCCC AGCCACTGGC GCTCCTTTCA
GAACCAAGTT TTCAAGACTT CATGTGATTT ACTATCTTCC AACTAGTAGA GAAAATCAAA
ACGGCACTGA AGCTGAAGCT GGCAGAGCAC GAGGTTCACT CACTGGCTAA TGCCTCACTT
CCGTGCACAG CCTGGCAGCC CCAAGGCTCC AAATAAAACT TGCAGCTGAG CTACAAATCT
CTCCACTCAC CAAAGAGCCA AACCTTCAAG GTTAAAGTCT GAGGCCAAGG GTGCGTGCGG
CTCAATTATA GCATCGCGTA AAAGGATTTT CCAAAGATCT AGTGGGTCCC TGGGATGGAA
GGGAAAGCAG ACTGGGCGGG TATCATGCTC CCATTTTAAG TCTGGGGTAG TTGATGTACC
CAATGTCACA TAACTGTTTT TAGGAGCTGG GATTCAGTAA GCAGGTGCTC CTGCTGTCTT
TCAAGGAGAT GAACCATGAA GACAGTCGCG CCTTCTGGCT GGCCAGGCGC AGGTCAAGAC
AGCGCCTTGG AAGAAGGGAT GGGCAGTGGG GAGGAAGGTG GTGCAGGAGC CTGTGGAGGG
GGTGGAGGGC CAGGTTCTGA CATGGCCTGC TCCAGCCTGC AGCAGCTGCT ACCCACACCC
CCACCCCCAG GGCCAGCACA GAATGGGGCT GGAATGAAGC TGCCATCCCA ATTCCCAGTG
TGATTTTGGT ACAAAGGTTA ATCACACCCA GAAAATGCTT ATCAGAGACC ATAAATAACC
GTGTTGTTGA AGCTCAGTTT GGTCTTTAAG GCACAAAAGT GCAATTTCCT TTGATGTGAT
GATTCGGTAA TTAACCTTTT GGGGAGGGTC CTGAAGGCCA CTGGAGGGAG GGAGGGGTGG
CCTGGAGGCA GCTGAGGCAT CTCAGGCCTG GTTCCTACAA GTTCCTGGGG AACAGGGAAA
AGGGAGCCTG TTTTCTCTCT CCTTTCACAG CTAGCACTGA TGCTCAGCTT GGGAGAGTCC
CCAGAGCCCC CAGGGGTCAG GGACCGCACT GGCCTTCGGC AGAGCCACAG CTGGACAGCC
ATCCTTCAAC ACGGTCCCTC GCCCTTCTGC TGAGCCCCAA GGCAGTCCTG tcactggctg
tgtggctttc ggtgagtcac ctgactcctc tgagactcaa catcctcatc tgtgaagtgg
gggcaatGTG GTCTTTCTTA CCCACTTCAC TGGGAGATGG ACCATTTGTG GGGAACCAGG
AGACCTGCAT CTGCAATATA AAGTCCCTAC AAGCATCGGC AGGGTCATCA GCCCAGCTTG
GCTGTTCTGG GCCCCAGAAT GCACCTCCAC TGCAGAGGGC TGGCCTGGAG CAGCTTTCCT
GATGTCCCTT CCGGGCCCTG GGGGAGGCCC CGCAGGCTGG ACCTGGCCTC TCCTCATCTT
CCGCCAGGTC CTTCTGCCTT CTCAGGAAGC CTTGCTTTGG GAAGGAGTTT TTCCATTTAA
AAGGTAAGTT GCTTTTTAGG TTTGAAATCC ACTGGATTGA GTGGTGTCTC CAACCCCCTT
GGTGGTCTG
>rs242924, SEQ ID NO: 3
AAGGTGAGGT TCAAGAGGGG TTGAGGGTAT GCTCATCCCT GTGGCTACCA GTACCCACCC
CTAGCCGGGA CATGACCAGG CACCTTCTGT GAGCCAGCAC AGGGACCCAG TGTGCCTTCT
CCAAGTCCAG GGCTTGTTCC TCTGCCCCCA GGCTCTCCCT AGTGAAGGAA CAGCTGGCCA
GGAAGCAGCT ACCATGAGAT GTGGAGGCCT CCTGTTCCCA CAGAGCCACC TTCAGAAATG
AAGGCCTCCT GCCCGCCTGC AGACACTTGC GTCTGTTTCT TTGCAAGCAT GCGTCTTCCA
CTCTGCCTCA TCGCACATGC ATTTGTGGAT TCGATGCCCA GACTAGCCTG TGAGCCCCCT
TGTTTCCCCC AGTGCCCTGC GTGGGGCCTG CTGTGTAGTA GGTGCTTCAT GAAAGAGTTG
GTGTGAATCA AGGAGAGGCT TGAAGACTTA AATAGAAGGT CCACAAGCCT TCCAAAGACA
CTCAGGTGCA GGGACCCTCT
CATTTTTGCC CAGCAGCAGC CATGCCCAGG ACCACACCCA AAGTTTTAAA AGACATGCCC
TAGGCAGCCC AAACACCTAG ATTTGGGTCC CAGTCACTGC CATGTACTGG CTGCATGGTG
CCATGGGGGT TATGTAACTC TCCCATGCCT CAGTTTCCTC AACTGTACAA ACGTTCGGCT
TTACACTCTT GTGAGGATGA AACGAGATGA TTATGCAAAA GCACTCTGTA AATGGTAAAG
TGACACAGAT ATTAATAATA TTTGCAGCTA TGCTGTCTTT TCTATTGGAG AGATACAGAG
CACGGCGCTG GAGACCACAA CCAGAAGCTG GGCTGCCCGT GCTCCACTCA GGGCTCTGGC
GCTTACTAGT TCTCTGTGCC TCAGTTTTCT TATGTGTAAA ATGGGGGCAA CAGCACCTAC
TTCAGAGGGA CATTGTGCAA ATTGGGTTAA TACAGCTCCA GTCCTTGATC AGTGCCTGGC
ACGGGTTAGC ACCGTGGAGT
>STMN1, SEQ ID NO: 4, GenBank Accession Number: NM_001145454.2

| | |
|---|---|
| 1 | gctctcggcc aatgcggagc cccgcgcgga ggtcacgtgc ctctgtttgg cgcttttgtg |
| 61 | cgcgcccggg tctgttggtg ctcagagtgt ggtcaggcgg ctcggactga gcaggacttt |
| 121 | ccttatccca gttgattgtg cagaatacac tgcctgtcgc ttgtcttcta ttcaccatgg |
| 181 | cttcttctga tatccaggtg aaagaactgg agaagcgtgc ctcaggccag gcttttgagc |
| 241 | tgattctcag ccctcggtca aaagaatctg ttccagaatt ccccctttcc cctccaaaga |
| 301 | agaaggatct ttccctggag gaaattcaga agaaattaga agctgcagaa gaaagacgca |
| 361 | agtcccatga agctgaggtc ttgaagcagc tggctgagaa acgagagcac gagaaagaag |
| 421 | tgcttcagaa ggcaatagaa gagaacaaca acttcagtaa aatggcagaa gagaaactga |
| 481 | cccacaaaat ggaagctaat aaagagaacc gagaggcaca aatggctgcc aaactggaac |
| 541 | gtttgcgaga gaagatgtac ttctggactc acgggcctgg ggcccaccca gcacagatct |
| 601 | ctgctgagca atcttgtctc cactctgttc ctgccctttg cccagccctg ggcctccaat |
| 661 | ctgcattgat tacctggtct gatctctctc accatcacta ggtacttaat aaatatttgc |
| 721 | tgttgatgat agcaatgacc ttgagactga tgaacagtct ggccaagagg atccttgatg |
| 781 | tggaagatag aaaaagcctt tggggtcagg cagacttgga ttctaatacc agccagttct |
| 841 | gcttgctgtg tctgagcctc agtttactca tctgtgaaga ggaggtagca agaatgaaaa |
| 901 | tgcctgcctt gtggtttgtt gtaaggacag acactgccaa cgtagagggc ccagcagctc |
| 961 | acagaccagt tgctctgaga gcagaccact cttgccttga tggtagggaa ctatttttgt |
| 1021 | gcgtggcaag tgggacctta ggaaggaagg caactgtgag gcttctgaga aggaccctac |
| 1081 | acaagggagt ttcctcccag ggcaggtgaa tggagagggt ggcagaagcc tacgggaagg |
| 1141 | ggtcacaggg atcagctaga gagtgccacc acccttcctg gggaatgcag ggcaaggtcc |
| 1201 | ctggtgggag ttttcctggg aagccaaaga agcgcccaac aaagacagaa tcaacatttg |
| 1261 | ggtacctttg gtacccagag gcagcaatgc caactacaac cacactggaa gaagaagacc |
| 1321 | ttctccgcat agattctctg atctcttcct ccttcatggc accagccctg gggaaccagc |
| 1381 | atggtgggga aataatgaag ctggaataca accacttaca gacttcacaa cctcctcctg |
| 1441 | tagataccaa agggatttta ggatcacatt ttatttctca cctgagcaag aaaagctaca |
| 1501 | ggagcatctc aagcagaggg caggagtctc cagaggagtt caaggggctc tggcaagaaa |
| 1561 | aatcaagggg ctgtgttcaa gaactggctc ccttggtgat tgtattacga agcccatgtg |
| 1621 | tgctggatgc tgatgaaatt gctgccaaat gcctgtgcag ccttggcaag gccctttatt |
| 1681 | tctctgggtc tccatttctc tctctctttt tttttttttt ttttttttga ggcagagtct |
| 1741 | cactctgtcg cccaggctgg agggcagtgg cgtgatctcg gctcactgca agccccacct |
| 1801 | tctgagttca cgccattcta ctgcctcagc ctcccgagta gctgggacta caggcgccca |
| 1861 | ccaccacgcc cggcttattt tttgtatttt tagtagagac ggggtttcac cgcattagcc |
| 1921 | aagatggtct cgatctcctg acctcgtgat tcacccacct cagcctccca aagtgctggg |
| 1981 | attacaggca tgagccactg cgcccggcct gggtctccgt ttctctagct gtgaaatgac |
| 2041 | tgttctaaaa gagccctgcc ggactttggc agtctgtaag aagacctgag ttcttctctc |
| 2101 | agttccaagc aggaaaattg aacataccct gagcccagag cctgcaacaa actctgggca |
| 2161 | gcctcaggaa gtcaggcagt gaagtcggaa aaatgatctc ttctgtatag ggagaaaata |
| 2221 | aaagtttaaa aatttgtaaa aaaaaaaaaa a |

>rs182455; SEQ ID NO: 5
CTTCAGAAGA GAGTGGATCT TGAAGTTCGT TTTAGGAAGC TTTCGAGAAG GGCGATTCTG
CCCCTCTCCA AGGTGAAGTG GCAAAACTGT TGTCTCTCCT TACAGTTGAA AGCCCTTCCT
ATTCCAAGGC TTATATAGGA CACCCACAAA AGCTGACAAT AATGAGCTGT GATCTGGCTC
TTGGGAAGGC AGCCCCACCC TAAAGAACAA GGATCAGACT GGGTGTGGTG GCTCATGCCT
GTAATCCCAG CACTTTGGGA GGCTGAGACA AGAGGATTGC TTGAACCCAG GAGTTCGAGG
CCAACCTGGG CAACATAGTG AGACCCTGTC TCAATTTTTT TTAAGTCAAA TAATAATAAT
AATAATAATA ATAATAATAA TAATAATAAT AATAATAAAA GGACAGGAAT CCCACCTCAA
AAGCACCTTT TTATTCAAAG GCTCATTCTA GGGGAAACTT TAACCCAGCC CTCTCAGTCC
TCAAGCAGCT GTGAGGAGCT
GGAACATTGT CACAGACTGG CAGAACCAGG CATCTCCCAA CAAACCCAGA AGCCAAGTGG
CATTGCTAAA AAAGATTAAA AATGAACATG AGTTCTAACA TCTACGTTCC TTGCTATGGT
TTCCATGGAA ATAGACATCT GCTCTAGGGA GAAAGAAAAT GTGGGGGGAA GCAGTGTAAG
TGCTTTCAAA TTATCAGTGT TTTGAAAGAT TTATTTCTTT TTTCTGTCAC AACATTAATA
GTCAGGAATG TGAGACTTTT GTGTGGCAAT TTATTGTACT TAAAAAAATG CATTAAATGA
GTGGGACCTT CCTCTGTGAA CCTCCAGATC AAATACAAAG ACTTCAAGAT AAAAAGCCTG
TTCAGTGGGT AAGGACCTCT TGGGCTTGGG TTCCTTGCTC ATTTGTGTTG CCTGCCTATC
TGTCGCTTTC CTGAGGGGTC TCATGCCCAT CCTTCTCCAA CTTTTCTAAT GATGTCTGAG
ATTCTGTTCA GAAAAATGAC

Statistical Analyses

All statistical analyses were conducted in SPSS software (IBM Corporation, New York) using analyses of variance, t-tests and related general linear models. Linear mixed models were used to assess treatment-related changes and moderation of treatment symptom response in order to fully account for study drop-outs in an intent-to-treat framework.

Assessing Treatment-Related Changes

For each outcome, a random-intercept model with maximum likelihood estimation was used to assess differential change in measures over time. A fixed and random intercept, a fixed variable corresponding to time (coded as 0 at baseline and 1 at post-treatment), and an effects-coded fixed variable for group were entered into the analysis. Model terms included the effect of time and the group×time interaction. Group was not identified as a model effect consistent with randomization of baseline characteristics. The effect of interest was the Type III Sums of Squares F statistic corresponding to the group×time interaction.

Assessing Moderation of Treatment Changes on Symptoms

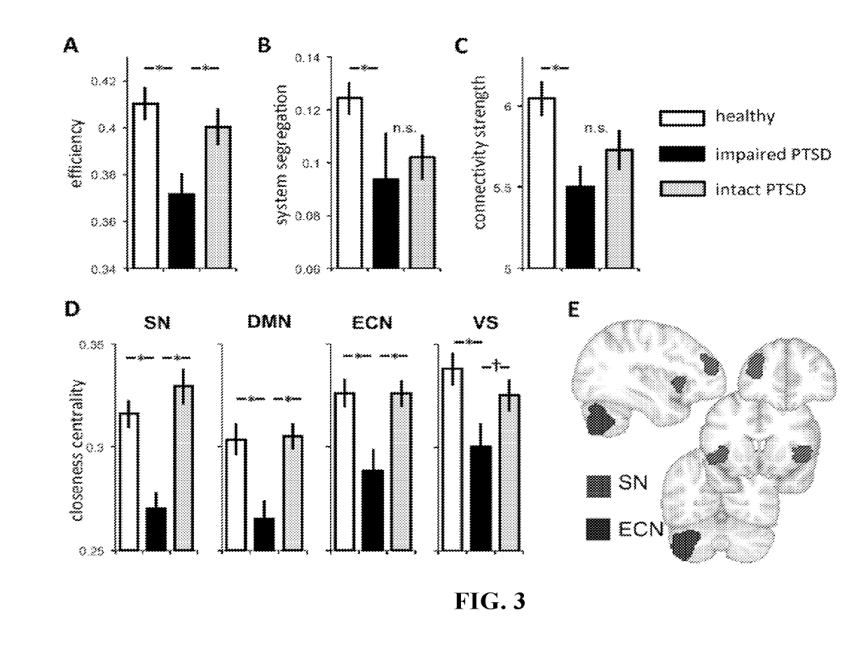

Efficiency and verbal memory, and their interaction, were entered as moderators of treatment effects on CAPS total scores. For each model, a random and fixed intercept, fixed variable corresponding to time, fixed variable corresponding to effects-coded group, and a fixed variable corresponding to the moderator(s) of interest were entered into the analysis and estimated using maximum likelihood. Verbal memory delayed recall performance was dichotomized as ≥ or <95% accuracy (the median value) due to a non-normal distribution (see FIG. 3), while efficiency was examined as a continuous variable. The following effects were specified in the model: time, group×time, verbal memory, efficiency, time×verbal memory, time×efficiency, group×time×verbal memory, group×time×efficiency, and group×time×verbal memory×efficiency. The Type III Sums of Squares F statistic corresponding to this latter 4-way interaction was used to assess a conjoint moderation effect of verbal memory and network efficiency on differential symptom change by treatment group. A similar analysis was used for the combined efficiency/memory biomarker (effects-coded), resulting in a model of time, group×time, biomarker, time×biomarker, and group×time×biomarker.

### Example 2: Establishing Brain-Behavior-Diagnosis Relationships

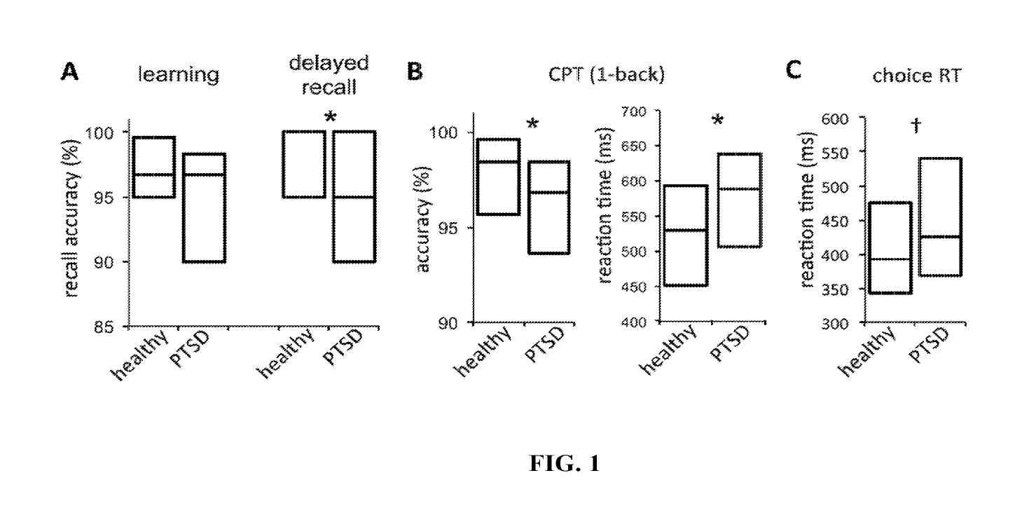

Three tasks were administered to participants in Study 1 to characterize the neurocognitive functions consistently impaired in PTSD² (Table 1, 2)—a word list learning task assessing verbal learning and delayed recall, a 1-back continuous performance test (CPT) assessing attention/working memory, and a choice reaction time (RT) task assessing processing speed. In Study 1, the PTSD group (N=59) showed deficits in delayed recall, speed and accuracy in the CPT task, relative to trauma-exposed healthy participants (N=36; FIG. 1 and brief description for statistics).

TABLE 2
Trauma type details for Studies 1 and 2 (percent of PTSD
participants endorsing a category for their index trauma).

| Civilian/military trauma type | Study 1 |
|---|---|
| Natural Disaster/Fire/Explosion | 8.5 |
| Physical Assault | 23.7 |
| Assault with Weapon | 6.8 |
| Sexual Assault | 33.9 |
| Combat/War Zone Exposure | 10.2 |
| Injury/Illness/Suffering/Death | 16.9 |
| military trauma type | Study 2 |
| Intentional Use of Force (Torture, Physical Assault) | 13 |
| Accident (Vehicle - Rollover, Collision) | 2.2 |
| Perceived Elevated Threat of Attack | 10.9 |
| Small Arms Fire/Fire Fight | 15.2 |
| Sexual Assault | 2.2 |
| Killed in Action | 4.3 |
| Wounded or Dead Bodies/Body Parts | 8.7 |
| Explosion | 43.5 |

Next, rsfMRI was used data to construct undirected weighted graphs based on correlation strengths between regions within cognitive networks. Multiple percentile thresholds (see Methods) maximized graph robustness²² and ensured equal connection density across participants. Global efficiency (i.e. the average inverse shortest weighted path length) quantified overall capacity for network integration, while the system segregation index (i.e. the degree to which nodes can be parsed into clearly delineated groups) quantified network segregation. The latter was done based on widely-accepted a priori definitions for the ECN, DMN, SN, and VS network modules (FIG. 2A, Table 3)⁹. Patients with PTSD had lower average network efficiency (FIG. 2B) and network segregation (FIG. 2C) across the three graph thresholds. Moreover, the tight inverse relationship between integration and segregation normally seen in healthy participants is not evident in patients. Average connectivity strength across the network was reduced in PTSD (t=2.73, p=0.008). There was greater reduction in within rather than between-network module connectivity, with the location of connectivity decreases relating differentially to efficiency and system segregation (FIG. 2D, E brief description).

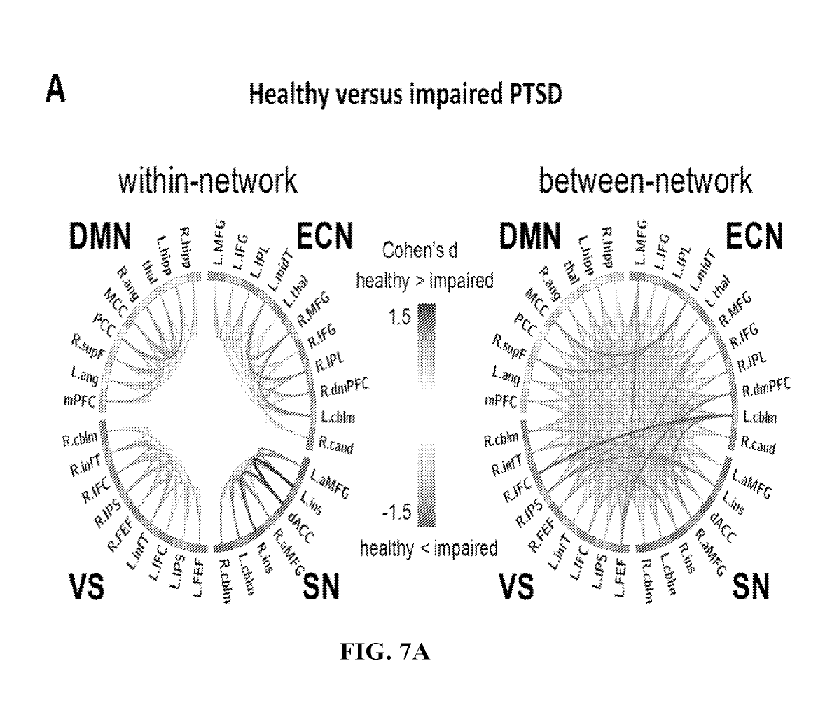

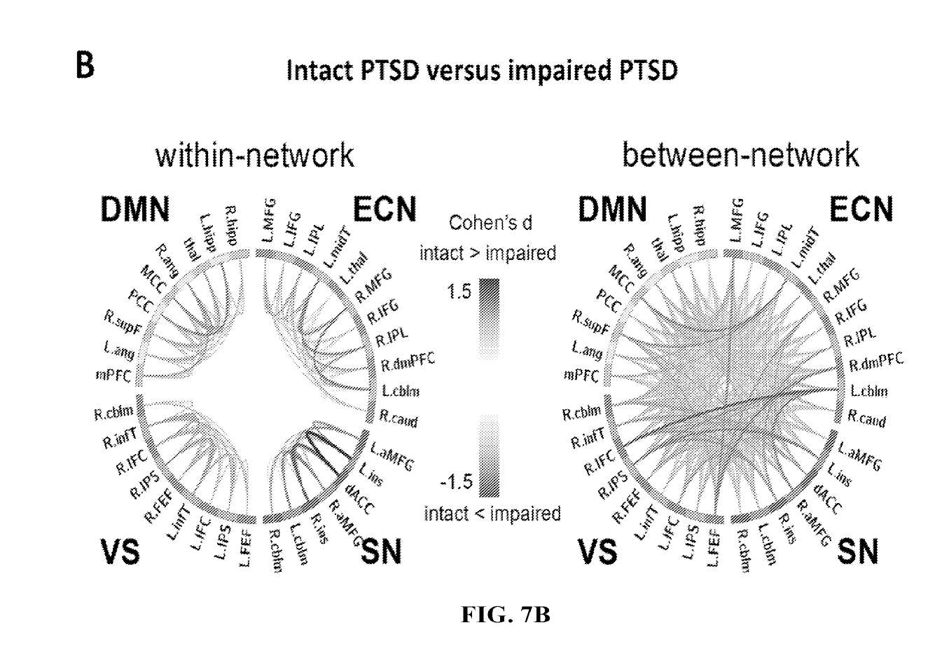

Next the relationship between cognitive deficits and network abnormalities were examined. It was predicted that impairments in mental processes requiring more interactions between networks (i.e. verbal learning and memory) would be most strongly associated with lower network integration or greater segregation^{9,23,24}. Analyses revealed a strong and specific relationship between impairments in memory and network integration. Specifically, only patients with impaired delayed recall showed decreased cognitive network efficiency with respect to healthy subjects or patients with intact memory (FIG. 3A), even if unthresholded graphs were used (FIG. 7). No significant memory-based grouping effects were found for system segregation and average network connectivity strength (FIG. 3B,C). Importantly, controlling for either system segregation or overall connectivity strength did not affect the relationship between memory and efficiency (p's<0.008). Controlling for age, IQ, or motion also did not eliminate the relationship between group and network efficiency (p's<0.0⁴⁴), despite the patient subgroups' ages differing by ˜10 years within a non-geriatric range (Table 4). Notably, performance on the CPT task was not related to either efficiency or system segregation.

TABLE 4
Demographic and clinical characteristics of participants by memory-based
groupings. The only variable that differed between groups was age.

| | Study 1: memory-based grouping | | |
|---|---|---|---|
| | healthy | impaired PTSD | intact PTSD |
| Age (yr) | 34.6 (11.7) | 44.4 (12.7) | 33.9 (8.4) |
| Education (yr) | 15.3 (2.4) | 15.1 (2.6) | 14.9 (2.6) |
| Gender (% female) | 53% | 64% | 62% |
| WASI intelligence | 112.3 (13.1) | 107.5 (13.4) | 112.0 (9.4) |
| quotient (IQ) | | | |
| self-report | | | |
| Beck Depression | 1.6 (1.9) | 23.9 (10.1) | 21.7 (8.4) |
| Inventory | | | |
| WHOQOL physical | 87.6 (11.4) | 47.7 (22.4) | 57.1 (17.0) |
| health | | | |
| WHOQOL | 75.5 (11.6) | 41.7 (40.1) | 40.1 (15.7) |
| psychological health | | | |
| WHOQOL social | 77.1 (19.8) | 36.9 (26.9) | 34.6 (22.1) |
| relationships | | | |
| WHOQOL | 79.4 (14.8) | 55.6 (24.0) | 52.6 (20.0) |
| environment | | | |
| DSM-IV | | | |
| clinician scales | | | |
| CAPS total | 2.4 (3.4) | 69.1 (19.3) | 66.6 (14.2) |
| CAPS re-experiencing | .7 (1.5) | 17.8 (6.6) | 17.7 (6.0) |
| CAPS avoidance | .7 (1.6) | 29.2 (8.9) | 27.2 (8.0) |
| CAPS hyperarousal | 1.0 (1.8) | 22.1 (6.7) | 21.7 (5.3) |

Age: impaired versus intact PTSD p = .01

| | Study 2: memory-based grouping | | |
|---|---|---|---|
| | healthy | impaired PTSD | intact PTSD |
| Age (yr) | 30.7 (8.5) | 35.6 (6.9) | 31.2 (7.1) |
| Education (yr) | 14.4 (3.3) | 14.5 (3.0) | 14.0 (3.6) |
| Gender (% female) | 9% | 7% | 10% |
| WASI intelligence | 108.7 (10.7) | 102.3 (11.5) | 102.6 (11.9) |
| quotient (IQ) | | | |
| using antidepressants | 0% | 31% | 27% |
| (% yes) | | | |
| self-report | | | |
| Beck Depression | 2.4 (3.7) | 21.6 (11.9) | 17.3 (11.2) |
| Inventory | | | |
| WHOQOL physical | 84.2 (14.1) | 58.0 (16.8) | 61.2 (18.3) |
| health | | | |
| WHOQOL | 79.0 (16.1) | 45.5 (18.8) | 56.8 (18.3) |
| psychological health | | | |
| WHOQOL social | 70.2 (18.1) | 45.5 (34.6) | 46.5 (19.6) |
| relationships | | | |
| WHOQOL | 72.9 (14.6) | 54.9 (13.8) | 59.1 (18.6) |
| environment | | | |
| DSM-5 clinician scales | | | |
| CAPS total | 2.9 (2.8) | 32.4 (10.1) | 30.8 (11.0) |
| CAPS re-experiencing | .8 (1.3) | 8.4 (3.3) | 6.9 (3.7) |
| CAPS avoidance | .18 (39) | 4.1 (1.7) | 3.7 (1.3) |
| CAPS hyperarousal | .24 (.56) | 10.5 (4.9) | 10.5 (5.4) |
| CAPS negative | 1.7 (1.6) | 9.4 (3.3) | 9.7 (3.1) |
| cognitions | | | |

Age: impaired versus intact PTSD p = .05

To understand network integration at the brain regional level closeness centrality (inverse of the weighted path length between that region and all others) was quantified for each region, since this measure is both mathematically similar to efficiency and correlates highly with it (r=0.942, p<0.001). An effect of the memory-based grouping on average closeness centrality across regions within the ECN, SN and DMN network modules was found, and a trend for the VS module (FIG. 3D). These relationships remained significant after controlling for participation coefficient, which a the nodal metric of segregation closely related to the overall system segregation index used above (p's<0.008)⁹. After false discovery rate (FDR) correction, the greatest reductions in closeness centrality were in the left anterior insula and left anterior middle frontal gyrus (SN) and left lateral cerebellum (ECN, FDR q's<0.05) with a trend for the right insula (SN; q=0.054; FIG. 3E).

### Example 3: Replicating Brain-Behavior-Diagnosis Relationships and Use as a Biomarker

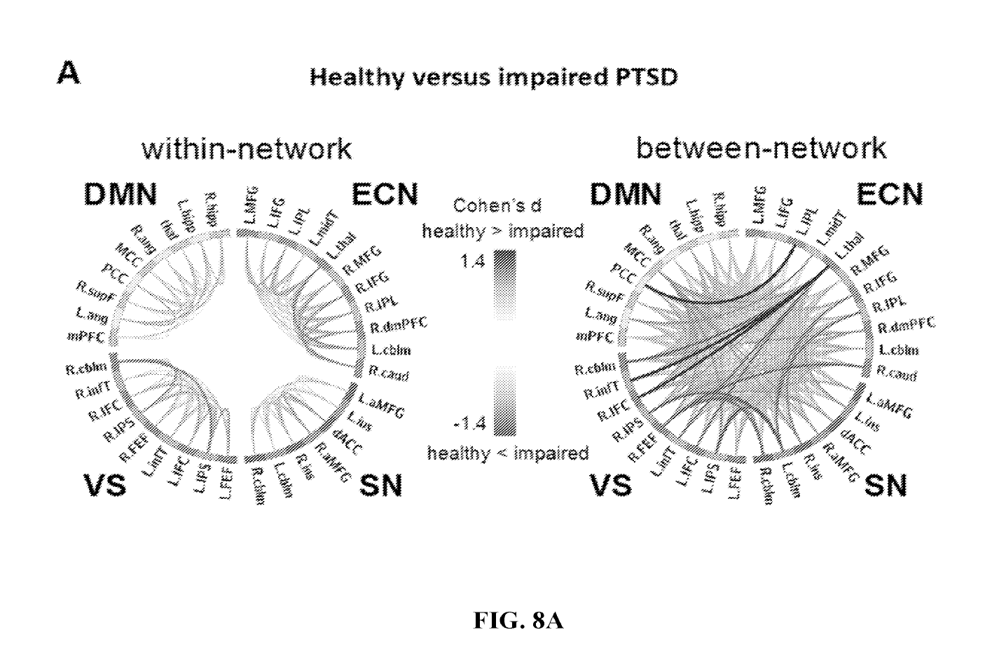

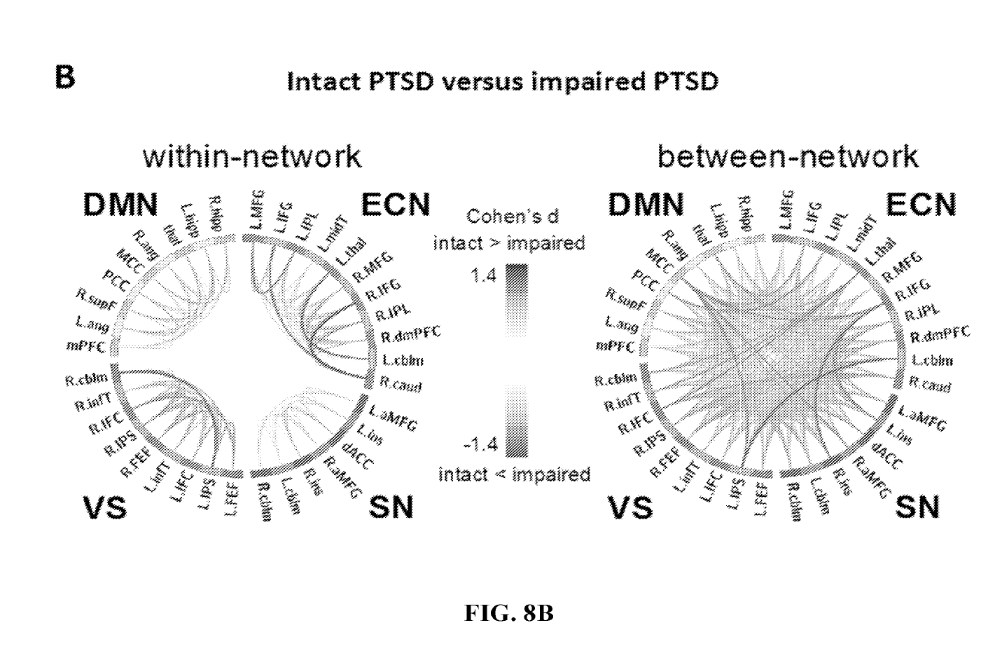

Participants in Study 2 (a two-site study; Table 1, 2) were divided into healthy controls (N=23), PTSD participants with impaired delayed recall (N=16) and PTSD participants with intact memory (N=30) by applying the cutoffs determined based on Study 1. As in Study 1, a significant effect of group on network efficiency was found (FIG. 4A), controlling for study site and age (since impaired patients were slightly but significantly older; Table 4), wherein only cognitively impaired PTSD participants had lower cognitive network efficiency. Similar effects were found for unthresholded graphs (FIG. 8). No effect was found on system segregation (F=0.07, p=0.934) and controlling for either system segregation or medication use did not affect the impact of group on efficiency (F's>3.93, p's<0.025). Thus, Study 2 directly replicated Study 1, despite substantial differences between them in diagnostic criteria (DSM-IV versus DSM-5), gender preponderance (female versus male), sample population (largely civilian versus combat veteran), site (Stanford versus Stanford and NYU) and data acquisition method (spiral at one site versus echo-planar imaging at two sites). Strikingly, when applying the same memory cutoff to patients who had TBI but not PTSD, there was no difference in cognitive network efficiency (FIG. 4A). Thus, efficiency is not low simply when memory is impaired. Rather, there is specificity for the combination of memory and network integration impairments in PTSD.

The clinical utility of the findings was tested. A receiver operating characteristic (ROC) analysis was used to find the optimal efficiency cut-point for discriminating between the healthy and impaired memory groups in Study 1 (FIG. 4B), which occurred at the 61^{st} percentile in the healthy participants' efficiency distribution. Applying these cut-points to Study 2, impaired memory or efficiency each alone distinguished between PTSD and/or TBI, but only at modest specificity (sensitivity is less relevant for a subgroup biomarker; Table 5). This was due in part to the fact that memory impairment in TBI, unlike PTSD, was not associated with decreased efficiency (FIG. 4A). By contrast, a combination biomarker of memory and efficiency was significantly more likely to detect the PTSD group than either healthy or TBI participants with a striking specificity of 94-95% (FIG. 4C). The combined biomarker defined a subgroup of 30% of PTSD participants (i.e. 30% sensitivity) who can be characterized as having cognitive dysfunction across both network and behavioral levels. Symptom levels between PTSD participants positive and negative on this biomarker were then compared, and no differences in symptoms of PTSD, depression, alcohol use or quality of life in either study were found (Table 6). Thus, the stratification biomarker identifies an objectively-defined PTSD phenotypic subgroup that is not confounded by clinical illness severity.

TABLE 6
Clinical comparison of efficiency/memory subtype positive
to subtype negative PTSD patients (uncorrected p-values).

| | |
|---|---|
| Study 1 | Study 2 |

self-report

| | | |
|---|---|---|
| Beck Depression Inventory | .951 | .393 |
| WHOQOL physical health | .163 | .997 |
| WHOQOL psychological health | .947 | .744 |
| WHOQOL social relationships | .337 | .294 |
| WHOQOL environment | .917 | .581 |
| AUDIT | | .431 |

DSM-IV clinician scales

| | |
|---|---|
| CAPS total | .944 |
| CAPS re-experiencing | .631 |
| CAPS avoidance | .683 |
| CAPS hyperarousal | .787 |

DSM-5 clinician scales

| | |
|---|---|
| CAPS total | .807 |
| CAPS re-experiencing | .208 |
| CAPS avoidance | .592 |
| CAPS hyperarousal | .774 |
| CAPS negative cognitions | .720 |

### Example 4: Prediction of treatment outcome

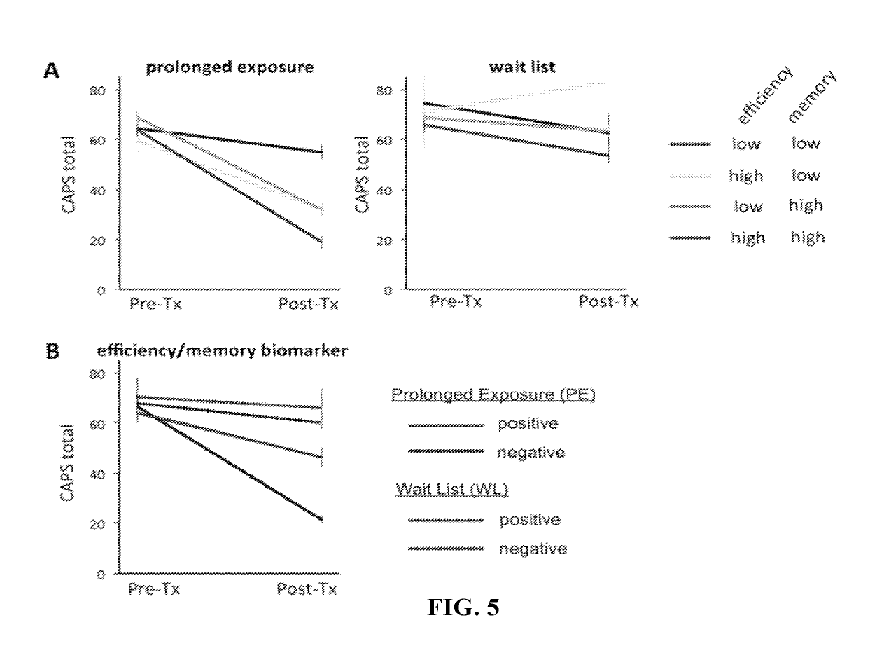

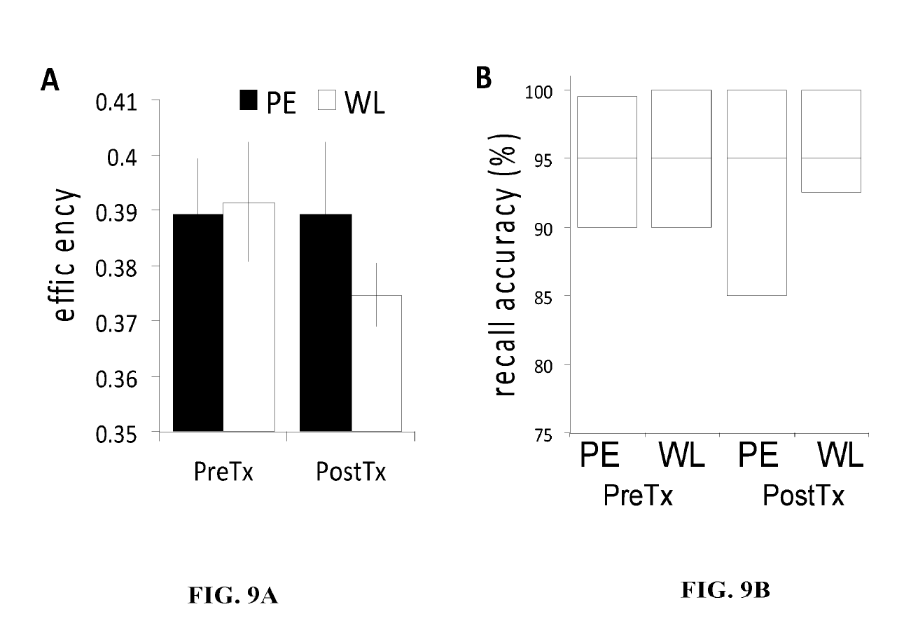

To establish further the clinical relevance of impairments in rsfMRI-derived cognitive network efficiency and memory it was examined whether these metrics could predict treatment outcome. Many of the PTSD patients in Study 1, as well as a small number of patients taking antidepressants, took part in a treatment study in which they were randomized to receive either prolonged exposure psychotherapy (N=36) or a wait-list minimal attention intervention (N=30; see Methods; Table 7). Trauma-focused psychotherapy, such as prolonged exposure, is considered the best treatment for PTSD and centrally involves memory-related processes^{21,25}. Analyses were conducted with linear mixed models to achieve a full intent-to-treat framework (see Methods). A significant interaction between treatment arm, memory impairment and efficiency was found in predicting treatment outcome (FIG. 5A). Outcome with exposure was worse in individuals with impairments in network efficiency and/or memory, but these metrics showed no relationship to outcome after wait list. Using the combined efficiency/memory biomarker definition above, a significant treatment arm×biomarker interaction was also found (FIG. 5B). This was driven by a large and significant difference between exposure and wait list for patients negative for this cognitive dysfunction biomarker, but failure of exposure therapy to differentiate from wait list in biomarker positive patients. By contrast to the prediction findings, exposure therapy did not alter either efficiency or memory (FIG. 9), further demonstrating the invariance of this core biological phenotype. In other words, network/memory-impaired patients neither benefitted symptomatically nor neurobiologically from treatment, while intact patients benefitted symptomatically and were indistinguishable from controls in terms of network architecture and cognitive function.

TABLE 7
Demographic and clinical characteristics of participants
with means and (standard deviations) for the intent-to-treat
analysis of treatment outcome and its moderation by
memory and efficiency.

| | Study 1 treatment sample | |
|---|---|---|
| | prolonged | |
| | exposure (N = 36) | wait list (N = 30) |
| Age (yr) | 34.4 (10.2) | 39.0 (10.4) |
| Education (yr) | 14.7 (2.2) | 15.2 (2.8) |
| Gender (% female) | 64% | 50% |
| WASI intelligence quotient (IQ) | 109.0 (9.1) | 112.8 (11.6) |
| using antidepressants (% yes) | 17% | 20% |

self-report

| | | |
|---|---|---|
| Beck Depression Inventory | 23.7 (8.7) | 23.2 (8.6) |
| WHOQOL physical health | 52.9 (18.7) | 52.7 (19.5) |
| WHOQOL psychological health | 37.7 (14.3) | 42.7 (14.6) |
| WHOQOL social relationships | 35.7 (25.4) | 33.0 (21.9) |
| WHOQOL environment | 51.9 (21.7) | 54.9 (21.1) |

DSM-IV clinician scales

| | | |
|---|---|---|
| CAPS total | 66.3 (15.2) | 71.4 (15.0) |
| CAPS re-experiencing | 17.5 (6.4) | 18.7 (6.0) |
| CAPS avoidance | 27.0 (7.9) | 28.8 (8.9) |
| CAPS hyperarousal | 21.9 (6.3) | 23.9 (4.9) |

### Example 5: Relationship of Regional Gene Expression to Network Integration

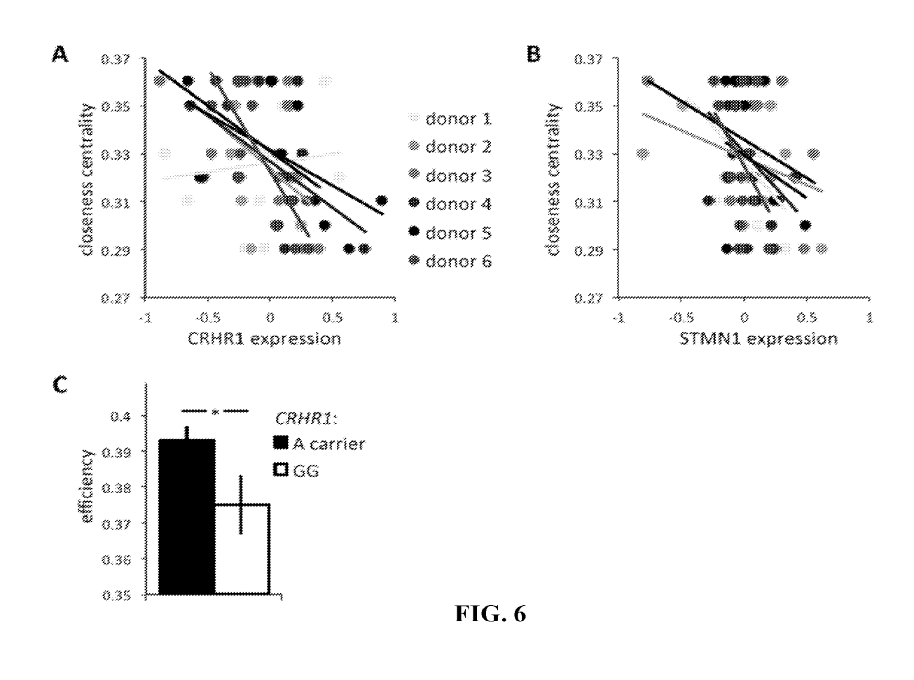

Next, it was examined whether genes related to PTSD (variants of which may contribute to cognitive dysfunction) are expressed in normal brain samples across multiple cortical regions in a manner that reflects that region's contribution to network efficiency¹⁵. Using expression data from many cortical regions across six healthy post-mortem humans in the Allen Institute for Brain Science (AIBS) data set^{13,26}, each microarray sample location was aligned to network regions (see Methods). Then, for each donor separately, the correlation between gene expression and each region's closeness centrality (derived from healthy participants) was calculated as was a combined p-value across donors. After FDR correction, two genes whose expression was inversely correlated with closeness centrality were identified: CRHR1 (a receptor for the corticotrophin releasing hormone) and STMN1 (the microtubule regulating protein stathmin; FIG. 6, Table 8). These correlations held even after controlling for regional participation coefficient values (a region-level measure of network segregation) and were evident in at least five of the six donors individually (FIG. 6 brief description). In a similar analysis, no previously identified functional-network related gene¹³ correlated with closeness centrality, even considering the greater number of genes in this set than the PTSD set (FDR q's>0.36; Table 9).

TABLE 8
Correlation of nodal expression of PTSD-related genes
with resting-state fMRI-derived nodal closeness centrality
(one-sided p-values, ***= FDR significant)

| gene | r | p (unc) | q (FDR) |
|---|---|---|---|
| ADCYAP1R1 | 0.16 | 0.093 | 0.241 |
| COMT | −0.02 | 0.438 | 0.456 |
| CRHR1*** | −0.51 | 0.00003 | 0.0007 |
| DRD2 | 0.07 | 0.289 | 0.395 |
| FKBP5 | 0.17 | 0.086 | 0.241 |
| APOE | 0.12 | 0.184 | 0.307 |
| CHRNA5 | −0.14 | 0.148 | 0.305 |
| DTNBP1 | −0.14 | 0.171 | 0.305 |
| GABRA2 | −0.08 | 0.332 | 0.410 |
| HTR2A | −0.30 | 0.011 | 0.095 |
| NR3C1 | 0.15 | 0.128 | 0.292 |
| RGS2 | −0.06 | 0.300 | 0.395 |
| SLC18A2 | 0.20 | 0.096 | 0.241 |
| SLC6A3 | 0.14 | 0.171 | 0.305 |
| SLC6A4 | −0.10 | 0.251 | 0.393 |
| SRD5A2 | 0.04 | 0.394 | 0.448 |
| STMN1*** | −0.39 | 0.0007 | 0.009 |
| TPH1 | −0.01 | 0.465 | 0.465 |
| WWC1 | 0.05 | 0.345 | 0.410 |
| PRTFDC1 | 0.19 | 0.085 | 0.241 |
| ANK3 | −0.20 | 0.070 | 0.241 |
| DRD4 | −0.28 | 0.015 | 0.096 |
| RORA | −0.05 | 0.412 | 0.448 |
| TPH2 | 0.18 | 0.080 | 0.241 |
| TLL1 | −0.08 | 0.283 | 0.395 |

TABLE 9
Correlation of nodal expression of functional network-
related genes (Richiardi et al., 2015) with resting-state
fMRI-derived nodal closeness centrality (one-sided p-values)

| gene | r | p (unc) | p (FDR) |
|---|---|---|---|
| ADAM23 | 0.09 | 0.247 | 0.443 |
| ALOX12 | 0.11 | 0.210 | 0.443 |
| AMDHD1 | 0.08 | 0.264 | 0.443 |
| ANKRD6 | −0.24 | 0.038 | 0.356 |
| ASGR2 | −0.10 | 0.239 | 0.443 |
| ATP6V1C2 | −0.21 | 0.080 | 0.356 |
| BAIAP3 | −0.24 | 0.039 | 0.356 |
| C3orf55 | 0.00 | 0.500 | 0.501 |
| CALB1 | −0.16 | 0.126 | 0.379 |
| CARTPT | −0.08 | 0.302 | 0.443 |
| CCBE1 | −0.05 | 0.396 | 0.468 |
| CCDC39 | 0.11 | 0.240 | 0.443 |
| CD163L1 | −0.15 | 0.148 | 0.388 |
| CD6 | −0.16 | 0.130 | 0.379 |
| CD70 | −0.02 | 0.437 | 0.470 |
| CDK1 | −0.14 | 0.156 | 0.388 |
| CDR2L | 0.03 | 0.440 | 0.470 |
| CNTN6 | −0.12 | 0.178 | 0.417 |
| COL5A2 | 0.01 | 0.453 | 0.470 |
| CPLX1 | 0.19 | 0.080 | 0.356 |
| CRYBA2 | 0.16 | 0.119 | 0.379 |
| CTXN3 | 0.08 | 0.240 | 0.443 |
| CXXC11 | −0.15 | 0.151 | 0.388 |
| CYP2C18 | −0.10 | 0.253 | 0.443 |
| DMRT3 | 0.00 | 0.458 | 0.470 |
| ENPP6 | 0.11 | 0.247 | 0.443 |
| EPN3 | 0.08 | 0.284 | 0.443 |
| FAM163A | −0.23 | 0.048 | 0.356 |
| FEZF1 | 0.19 | 0.079 | 0.356 |
| FZD7 | −0.02 | 0.448 | 0.470 |
| GABRA5 | −0.11 | 0.231 | 0.443 |
| GAL | 0.05 | 0.356 | 0.465 |
| GALP | −0.21 | 0.059 | 0.356 |
| GLRA3 | −0.19 | 0.075 | 0.356 |
| GMPR | 0.06 | 0.273 | 0.443 |
| GNA14 | 0.21 | 0.075 | 0.356 |
| GNGT2 | 0.11 | 0.208 | 0.443 |
| GOLT1A | −0.05 | 0.359 | 0.465 |
| GPR20 | −0.02 | 0.442 | 0.470 |
| GPR26 | −0.09 | 0.293 | 0.443 |
| GPR88 | −0.21 | 0.051 | 0.356 |
| GPX3 | −0.02 | 0.461 | 0.470 |
| GRP | −0.23 | 0.056 | 0.356 |
| HOXD1 | 0.07 | 0.376 | 0.468 |
| HPCA | −0.32 | 0.006 | 0.356 |
| HPCAL1 | −0.28 | 0.023 | 0.356 |
| HSD11B1 | 0.05 | 0.391 | 0.468 |
| IL13RA2 | −0.12 | 0.214 | 0.443 |
| IL33 | −0.07 | 0.293 | 0.443 |
| IQCJ | −0.21 | 0.074 | 0.356 |
| ISCU | −0.04 | 0.368 | 0.467 |
| KANK4 | 0.14 | 0.160 | 0.388 |
| KCNA1 | 0.19 | 0.084 | 0.356 |
| KCNA3 | 0.05 | 0.349 | 0.461 |
| KCNA5 | −0.08 | 0.302 | 0.443 |
| KCNC1 | 0.07 | 0.306 | 0.443 |
| KCTD15 | −0.12 | 0.235 | 0.443 |
| KLK1 | 0.01 | 0.424 | 0.470 |
| KLK8 | −0.06 | 0.327 | 0.446 |
| KRT1 | −0.17 | 0.108 | 0.379 |
| KRT31 | −0.03 | 0.455 | 0.470 |
| LAIR2 | 0.03 | 0.402 | 0.468 |
| LGR6 | −0.15 | 0.114 | 0.379 |
| LINC00238 | −0.16 | 0.124 | 0.379 |
| LINC00617 | −0.03 | 0.410 | 0.470 |
| LMOD3 | −0.02 | 0.400 | 0.468 |
| LRRC38 | 0.02 | 0.441 | 0.470 |
| LYPLA2 | −0.13 | 0.188 | 0.434 |
| MGP | −0.08 | 0.266 | 0.443 |
| MS4A8 | −0.09 | 0.260 | 0.443 |
| MYBPC1 | −0.13 | 0.160 | 0.388 |
| MYH7 | −0.14 | 0.157 | 0.388 |
| MYLK3 | 0.06 | 0.294 | 0.443 |
| NEB | 0.04 | 0.393 | 0.468 |
| NECAB2 | −0.27 | 0.023 | 0.356 |
| NEFH | 0.21 | 0.055 | 0.356 |
| NEUROD6 | 0.05 | 0.323 | 0.446 |
| NEXN | 0.14 | 0.133 | 0.379 |
| NGFR | 0.06 | 0.322 | 0.446 |
| NKAIN4 | −0.02 | 0.442 | 0.470 |
| NOL4 | −0.25 | 0.042 | 0.356 |
| NOV | −0.15 | 0.152 | 0.388 |
| NPBWR2 | 0.13 | 0.157 | 0.388 |
| NRP1 | 0.06 | 0.299 | 0.443 |
| NUPR1L | −0.17 | 0.129 | 0.379 |
| ONECUT2 | 0.06 | 0.386 | 0.468 |
| ONECUT3 | 0.20 | 0.077 | 0.356 |
| OR51E2 | −0.02 | 0.445 | 0.470 |
| PCP4 | 0.20 | 0.071 | 0.356 |
| PIRT | −0.11 | 0.215 | 0.443 |
| PLCH1 | 0.05 | 0.335 | 0.450 |
| PNMT | −0.23 | 0.046 | 0.356 |
| PRR15 | −0.18 | 0.096 | 0.368 |
| PRSS35 | −0.10 | 0.227 | 0.443 |
| PTGS1 | −0.04 | 0.397 | 0.468 |
| PVALB | 0.12 | 0.204 | 0.443 |
| PYDC1 | −0.20 | 0.086 | 0.356 |
| RBP4 | −0.17 | 0.120 | 0.379 |
| RBPMS2 | −0.07 | 0.276 | 0.443 |
| RHOBTB2 | 0.20 | 0.070 | 0.356 |
| RSPH9 | −0.19 | 0.093 | 0.368 |
| RTP1 | −0.23 | 0.046 | 0.356 |
| SCARA5 | −0.25 | 0.040 | 0.356 |
| SCN1B | 0.09 | 0.262 | 0.443 |
| SCN4B | 0.28 | 0.021 | 0.356 |
| SEMA3C | 0.06 | 0.297 | 0.443 |
| SEMA7A | 0.25 | 0.025 | 0.356 |
| SH3RF2 | 0.08 | 0.306 | 0.443 |
| SHD | 0.05 | 0.380 | 0.468 |
| SHISA9 | −0.24 | 0.040 | 0.356 |
| SIX3-AS1 | 0.00 | 0.501 | 0.501 |
| SLC16A5 | −0.06 | 0.322 | 0.446 |
| SLC16A6 | 0.03 | 0.422 | 0.470 |
| SLC20A2 | 0.23 | 0.043 | 0.356 |
| SLC22A10 | −0.22 | 0.042 | 0.356 |
| SLC39A12 | 0.07 | 0.289 | 0.443 |
| SLN | −0.15 | 0.132 | 0.379 |
| SNAP25 | −0.07 | 0.309 | 0.443 |
| SOST | 0.07 | 0.310 | 0.443 |
| SPHKAP | 0.05 | 0.328 | 0.446 |
| SV2C | 0.19 | 0.081 | 0.356 |
| SYT10 | −0.16 | 0.134 | 0.379 |
| SYT2 | 0.20 | 0.081 | 0.356 |
| TDO2 | −0.16 | 0.103 | 0.378 |
| TGFBI | −0.02 | 0.463 | 0.470 |
| TINCR | −0.12 | 0.204 | 0.443 |
| TLX2 | −0.17 | 0.097 | 0.368 |
| TMEM52 | −0.12 | 0.198 | 0.443 |
| TNNT2 | −0.04 | 0.342 | 0.456 |
| TRIM29 | 0.11 | 0.178 | 0.417 |
| TSHZ3 | −0.12 | 0.219 | 0.443 |
| TSPAN8 | 0.03 | 0.432 | 0.470 |
| WISP1 | 0.03 | 0.365 | 0.467 |
| WISP2 | −0.05 | 0.402 | 0.468 |
| WNT4 | 0.01 | 0.427 | 0.470 |
| ZCCHC18 | −0.27 | 0.028 | 0.356 |

Finally, it was assessed whether previously-characterized brain function-impacting polymorphisms in the two genes identified above (CRHR1, STMN1) were associated with reduced network efficiency or memory (see Methods). For CRHR1 the GG form of a two-polymorphism haplotype (rs110402 and rs242924) has been associated with impairments in cortisol, working memory (in interaction with early life stress), and fMRI responses to emotional stimuli²⁷⁻²⁹. Though less studied, a polymorphism in STMN1 (rs182455) has been associated with altered amygdala volume and cortical event related potentials^{30,31}. In this data, the carriers of the GG CRHR1 risk allele had lower network efficiency (FIG. 6C), which held even after controlling for system segregation (F=5.06, p=0.026). No significant effects of the STMN1 genotype were found on efficiency, nor were there significant effects of either genotype on memory (p's>0.3).

### Example 6: Breakdown in the Integration/Segregation Relationship in PTSD

In healthy controls, efficiency and system segregation in Study 1 were negatively correlated with each other as previously reported (r=−0.678, p<0.001) ⁴⁸, but this relationship was not observed in patients (r=−0.003, p=0.981; difference in correlation strength's: Fisher's Z=3.75, p=0.0002). This indicated a breakdown of the expected relationship between network integration and segregation in patients, allowing us to examine their distinct contributions to cognitive task performance and cognitive symptoms.

### Example 7: Brain-Behavior-Diagnosis Relationship with Unthresholded Graphs

To determine whether the efficiency finding somehow related to graph thresholding, the effect of memory-based grouping on unthresholded graph efficiency was examined. It was found that a significant effect of group (F=3.17, p=0.047), driven by lower efficiency in the impaired group than either healthy participants (t=2.81, p=0.007) or intact memory PTSD patients (t=2.11, p=0.022) but no difference between healthy participants and intact memory patients (t=1.03, p=0.308). See FIG. 7 for visualization of connectivity across all within- and between-network connections for the contrasts of impaired memory PTSD patients to healthy participants and impaired memory PTSD patients.

### Example 8: Lack of Brain-Behavior-Diagnosis Relationships for CPT Performance

Reaction times on the CPT were not related to either efficiency or system segregation (r's<−0.061, p's>0.581). There was no effect of group determined through a median split of accuracy in the CPT on either efficiency (F=2.28, p=0.108) or system segregation (F=2.68, p=0.075). The latter trend effect was related to significantly greater segregation in controls than intact patients (t=2.43, p=0.018) and a numerical advantage of healthy controls over impaired patients (t=1.72, p=0.138), but no difference between patient groups (t=0.158, p=0.875).

### Example 9: Efficiency of Unthresholded Graphs in Study 2

Similar to the results from Study 1, it was found that an effect of memory-based grouping on unthresholded graph efficiency in Study 2 (F=3.97, p=0.024), driven by lower efficiency in impaired memory PTSD participants than either healthy (F=5.73, p=0.022) or intact memory PTSD participants (F=5.49, p=0.024) but no difference between healthy participants and intact memory patients (F=0.21, p=0.609). See FIG. 8 for visualization of connectivity across all within- and between-network connections for the contrasts of impaired memory PTSD participants to healthy participants and impaired memory PTSD participants.

Examples provided herein disclose, for the first time, a central and specific role for network integration in cognitive dysfunction associated with PTSD, in a manner that replicated across clinical population, disorder definition, gender, fMRI acquisition method and site. Moreover, a combined biomarker incorporating impaired delayed verbal memory delayed recall and poor cognitive network efficiency (cutoffs defined in study Study 1 and tested in Study 2) demonstrated 94-95% specificity in detecting cognitively-impaired PTSD participants compared to either healthy or TBI participants. Furthermore PTSD patients with impaired memory and network efficiency saw no benefit from exposure therapy, the best-validated treatment for PTSD, compared to a wait-list control intervention. Cognitively intact patients had a robust response to therapy with many reaching remission (CAPS≤20)³². Finally, brain regional expression of one PTSD-related gene, CRHR1, was specifically related to a region's contribution to network integration, and the risk-associated polymorphism of CRHR1 in turn was associated with lower network integration. Taken together, these results advance a unified and mechanistic view of the network underpinnings of cognition and its impairment in PTSD, providing a robust brain circuitry-based biomarker for a novel cognitive subtype of PTSD.

Few resting-state network studies have addressed network topological mechanisms of cognitive impairments in neuropsychiatric disorders^{3,33}. Greater cognitive effort in healthy individuals has been associated with a more globally efficient network configuration³⁴, with more long-distance synchronization between brain regions²³. Greater network efficiency in healthy subjects has also been related to higher IQ^{24,35}. Another study found that greater network segregation predicted better memory⁹. However, prior studies have rarely investigated integration and segregation in tandem, have often looked across the entire brain and not focused on the organization of cognitive networks, nor related network topology to cognitive dysfunction, or have examined populations with more global cognitive deficits (e.g. schizophrenia, autism). Similarly, two prior resting-state graph theory analyses of PTSD have yielded conflicting results^{36,37}, with neither providing data on cognition nor cognitive networks specifically.

It was found that a specific mapping between the capacity to integrate across cognitive control network modules and the more complex behavioral deficit (memory), which furthermore characterized a subgroup within the broader clinical category of PTSD. By contrast, deficits in simpler behavior (attention and information processing speed) may reflect either other aspects of network function, may not relate to connectivity of cognitive control networks (e.g. sensorimotor systems) or quite simply may not be associated with a pattern of connectivity evident at rest.

As a potential molecular mechanism, a specific association between a region's contribution to integration and CRHR1 expression was identified, as well as a selective reduction in network efficiency in individuals carrying the CRHR1 risk-related polymorphism. In humans, a CRHR1 polymorphism that is associated with larger cortisol responses to the dexamethasone/CRH hormone test ^{27,28} is also associated with greater early life stress-related cognitive impairments in a 2-back working memory task that is more difficult than the CPT used here²⁷. In rodents, stress impairs memory and leads to dendritic remodeling³⁸. This effect is mediated by CRHR1, which is expressed on pyramidal cell dendrites³⁹, and deletion of forebrain CRHR1 protects animals from both stress-related memory deficits and dendritic remodeling³⁸. CRHR1 has effects on glutamatergic transmission^{40,41}, which may furthermore contribute to cognitive deficits and dendritic remodeling. It is speculated that these dendritic changes may contribute to the microstructural basis of alterations in network integration observed through rsfMRI. As CRHR1 was least abundant in regions with greater hub function (higher closeness) and CRHR1 is thought to be over-expressed in stress-related disorders^{42,43}, hubs and consequent cognitive functioning may be particularly susceptible to negative effects of increased CRHR1 expression.

While STMN1 expression followed a similar pattern in the brain, a less well-characterized polymorphism in this gene did not alter network efficiency. Given the role of stathmin in stress⁴⁴, emotional memory⁴⁵ and dendritic length regulation⁴⁶, a more thorough characterization of its genetic regulation may reveal a role in network integration. Overall, however, gene expression findings disclosed herein inform in a more fundamental way an understanding of the molecular basis of network function in humans, wherein expression patterns correspond to a region's role in network topology. This compliments recent findings of greater similarity in expression of other genes (not related to topology discuss herein) between functionally interconnected brain regions¹³.

Finally, the biomarker properties of impaired network integration and memory in PTSD are particularly striking. The field presently lacks an objective method for diagnosing psychiatric disorders, systems and methods described herein which indicate direct replication in Studies 1/2 and ˜95% specificity (comparing PTSD to healthy or TBI participants) as a biomarker for a novel cognitive dysfunction subtype of PTSD. The clinical relevance of this biomarker for patient stratification is further underscored by its ability to specifically predict psychotherapy outcome (noting that other imaging treatment studies rarely employ randomized control arms), and the fact that it is not confounded by illness severity. These findings are thus in line with a more mechanistic approach towards psychiatric disease pathophysiology, such as the National Institute of Mental Health's Research Domain Criteria⁴⁷, which aims to integrate across measurement domains. While further replication of the treatment prediction effects will be necessary, this biomarker may have direct clinical relevance in personalized treatment selection.

### Example 9: System for PTSD Diagnosis and Treatment Strategy

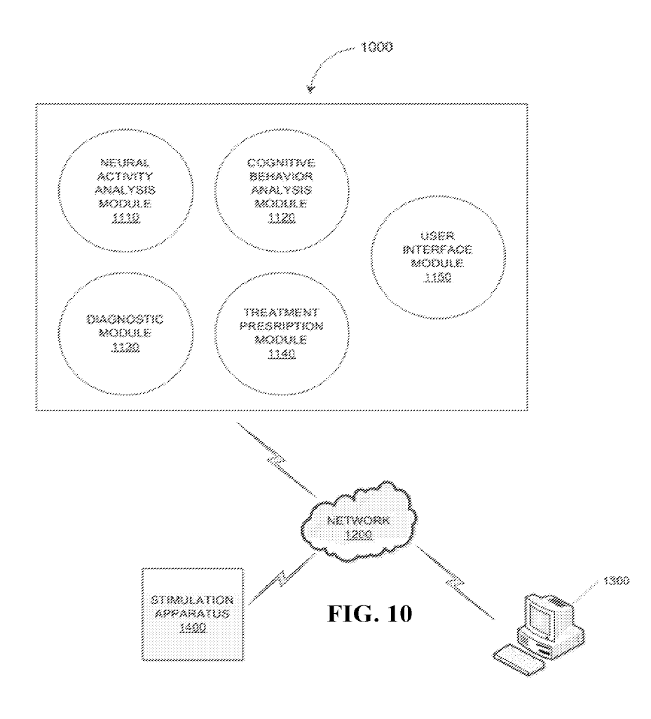

FIG. 10 is a system diagram illustrating a system 1000 for treating post-traumatic stress disorder, in accordance with some example embodiments. Referring to FIG. 10, in some example embodiments, the system 1000 may be realized in digital electronic circuitry, integrated circuitry, specially designed application specific integrated circuits (ASICs), field programmable gate arrays (FPGAs) computer hardware, firmware, software, and/or combinations thereof. The system 1000 may be configured to communicate with one or more devices (e.g., personal computers, workstations, tablet personal computers, and/or smartphones) via a wired and/or wireless network 1200. For example, as shown, the system 1000 may communicate with a device 1300.

In some example embodiments, the system 1000 may include one or more processors that implement a plurality of modules including a neural activity analysis module 1110, a cognitive behavior analysis module 1120, a diagnostic module 1130, a treatment prescription module 1140, a user interface module 1150, and a data collection module 1160. The system 1000 may include additional and/or different modules without departing from the scope of the present disclosure.

The neural activity analysis module 1110 may be configured to determine connectivity efficiency between different cognitive regions within a subject's brain by performing one or more neural activity analysis including, but not limited to, blood flow analysis, functional connectivity analysis, and structural connectivity analysis. According to some example embodiments, connectivity efficiency is part of a combined efficiency/memory biomarker usable in the diagnosis and treatment of PTSD.

In some example embodiments, the neural activity analysis module 1110 may use fMRI and/or NIRS to perform blood flow analysis and determine functional connectivity. Alternately or in addition, functional connectivity may also be determined using EEG and/or MEG. The structural connectivity analysis may be performed through a diffusion-weighted structural connectivity analysis.

In some example embodiments, the neural activity analysis module 1110 may be further configured to collect connectivity data for individual subjects. The connectivity data for a subject may be used to administer non-invasive brain stimulation on subjects requiring such treatments. As such, the neural activity analysis module 1110 may be configured to provide, over the wired and/or wireless network 1200, the connectivity data to a treatment apparatus 1400. A treatment apparatus may, for example, be adapted to administer non-invasive brain stimulation.

The cognitive behavior analysis module 1120 may be configured to analyze a subject's cognitive behavior. According to some example embodiments, cognitive behavior, particularly those related to memory, is part of a combined efficiency/memory biomarker usable in the diagnosis and treatment of PTSD.

In some example embodiments, the cognitive behavior analysis module 1120 may administer one or more cognitive and/or behavioral tasks adapted to allow determination of a complex cognitive behavioral deficiency (e.g., memory) in a subject. The cognitive and/or behavioral tasks may include, but not limited to, word learning, continuous performance, and choice reaction time. The cognitive behavior analysis module 1120 may administer cognitive and/or behavior tasks via, for example, the device 1300. For example, the device 1300 may provide a user interface for the subject to interact with the cognitive behavior analysis module 1120 and perform one or more cognitive and/or behavioral tasks. Alternately or in addition, the cognitive behavior analysis module 1120 may administer the cognitive and/or behavioral tasks through the user interface module 1150.

The diagnostic module 1130 may be configured to provide a diagnosis for a subject based on results from the neural activity analysis module 1110 and/or the cognitive behavior analysis module 1120. In some example embodiments, the diagnostic module 1130 may be configured to detect post-traumatic stress disorder in a subject based on the subject's combined efficiency/memory biomarker as determined by the neural activity analysis module 1110 and the cognitive behavior analysis module 1120. For example, the diagnostic module 1130 may determine that the subject suffers from post-traumatic stress disorder when the subject's biomarker indicates abnormal connectivity and/or cognitive behavior deficiencies.

In some example embodiments, the diagnostic module 1130 may provide the diagnosis via the user interface module 1150. Alternately or in addition, the diagnostic module 1130 may be configured to provide the diagnosis via the device 1300. For instance, the device 1300 may provide a user interface for a physician to interact with the diagnostic module 1130 including receiving and viewing the diagnosis provided by the diagnostic module 1130.

The treatment prescription module 1140 may be configured to provide a treatment plan for subject based on a diagnosis provided by the diagnostic module 1130. For example, the treatment prescription module 1140 may identify one or more treatments and/or devise a treatment strategy for a subject diagnosed with post-traumatic stress disorder (e.g., by the diagnostic module 1130).

According to some example embodiments, the treatment prescription module 1140 may further devise the treatment plan based on a subject's combined efficiency/memory biomarker as determined by the neural activity analysis module 1110 and the cognitive behavior analysis module 1120. In some example embodiments, the treatment prescription module 1140 may determine whether the treatment plan should include one or more types of treatments (e.g., psychotherapy, medication, non-invasive brain stimulation) based on the subject's combined efficiency/memory biomarker. In particular, the treatment plan may exclude psychotherapy for subjects exhibiting the efficiency/memory biomarker as such subjects tend to not benefit from first-line treatments such as psychotherapy. For those subjects, the treatment prescription module 1140 may provide alternate and/or advanced treatments including, but not limited to, medication and/or non-invasive brain stimulation.

In some example embodiments, the treatment prescription module 1140 may provide the treatment plan via the user interface module 1150. Alternately or in addition, the treatment prescription module 1140 may be configured to provide the treatment plans via the device 1300. For instance, the device 1300 may provide a user interface for a physician to interact with the treatment prescription module 1140 including receiving, viewing, and editing a treatment plan.

The user interface module 1150 may be configured to generate a user interface through which a user (e.g., a physician and/or a subject) may interact with the system 1000. For example, the user interface module 1150 may provide one or more user interfaces, such as graphic user interfaces adapted to provide visual outputs and/or receive inputs, for the administration of cognitive and/or behavioral tasks, the provision of diagnoses, and the provision of treatment plans.

In some example embodiments, the data collection module 1160 may be configured to collect data from the neural activity analysis module 1110 and/or the cognitive behavior analysis module 1120. For example, the data collection module 1160 may collect connectivity efficiency and/or complex cognitive behavioral deficiency data for one or more subjects. The data collection module 1160 may further collect diagnostic data from the diagnostic module 1130 and/or treatment plan data from the treatment prescription module 1140. According to some example embodiments, the data collection module 1160 may be communicatively coupled to a data store 1165 and may store some or all of the collected data in the data store 1165.

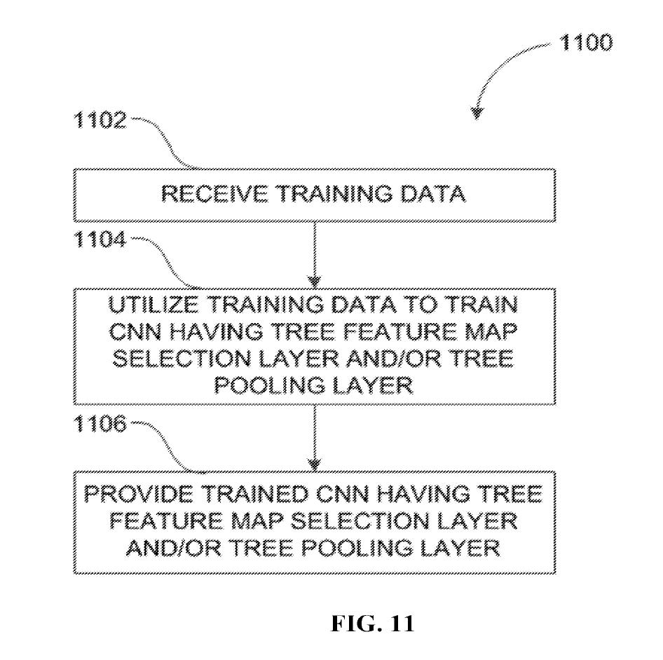

FIG. 11 is a flowchart illustrating a process 1100 for treating post-traumatic stress disorder, in accordance with some example embodiments. Referring to FIGS. 10-11, the process 1100 may be performed by the system 1000.

At 1102, the system 1000 may determine connectivity efficiency between a first cognitive region and a second cognitive region within a brain of a subject and/or determine a complex cognitive behavioral deficiency in the subject. For example, the system 1000 (e.g., the neural activity analysis module 1110) may determine a subject's structural and/or functional connectivity. Alternately or in addition, the system 1000 (e.g., the cognitive behavior analysis module 1120) may analyze the subject's cognitive behavior in order to identify a complex cognitive behavioral deficiency in the subject. For example, the system 1000 may administer cognitive and/or behavioral tasks (e.g., word learning, word learning, continuous performance, and choice reaction time) adapted to assess the subject's memory.

In some example embodiments, the subject's structural and/or functional connectivity as well as the subject's cognitive behavior may correspond to a biomarker. That is, the subject may be associated with a biomarker that includes the poor cognitive network efficiency and deficiency in the subject's cognitive behavior (e.g., memory deficiency). Thus, the system 1000 may determine whether the subject exhibits the biomarker by determining the connectivity between different cognitive regions in the brain of the subject and complex cognitive behavioral deficiencies exhibited by the subject. For instance, in some example embodiments, the system 1000 may be configured to screen the subject for memory deficiency. In the event that the result of the screening indicates that the subject exhibits memory deficiency, the system 1000 may then perform an imaging test, such as fMRI and/or MEG, on the brain of the subject in order to assess the subject's cognitive network efficiency. Alternately and/or additionally, the system 1000 may determine the subject's cognitive network efficiency by administering TMS. For example, the system 1000 can monitor, via EEG, the responses evoked by administering TMS to a right ECN of the subject.

At 1104, the system 1000 may provide a diagnosis for the subject based on the connectivity and/or the complex cognitive behavioral deficiency of the subject. For example, the system 1000 (e.g., the diagnostic module 1130) may determine that the subject suffers from post-traumatic stress disorder based on abnormal connectivity and/or complex cognitive behavioral deficiency present in the subject. In some example embodiments, the system 1000 may provide the diagnosis via the user interface module 1150 and/or through a user interface available on the device 1300.

At 1106, the system 1000 may generate a treatment plan based on the diagnosis for the subject. For example, the system 1000 (e.g., the treatment prescription module 1140) may identify treatments and/or devise a treatment strategy for a subject who has been diagnosed with post-traumatic stress disorder. The treatment plan may include one or more of psychotherapy, medication, and non-invasive brain stimulation.

In some example embodiments, the treatment plan may be generated based on the presence of the biomarker for poor cognitive network efficiency and complex cognitive behavioral deficiency. For example, the treatment plan may include non-invasive brain stimulation, which can be administered based on the connectivity between the different cognitive regions within the brain of a subject determined to exhibit the biomarker. According to some example embodiments, the treatment plan may also exclude certain treatments that are not effective for patients determined to have the biomarker. For instance, the treatment plan may exclude psychotherapy, which may be ineffective for a subject exhibiting the biomarker for poor cognitive network efficiency and complex cognitive behavioral deficiency. Alternately, the treatment plan for a subject exhibiting the biomarker may include at least one treatment in addition to psychotherapy.

At 1108, the system 1000 may provide the treatment plan. For example, the system 1000 (e.g., the treatment prescription module 1140) may provide the treatment plan via the user interface module 1150 or through a user interface available on the device 1300.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

Implementations of certain embodiments can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive track pads, voice recognition hardware and software, optical scanners, optical pointers, digital image capture devices and associated interpretation software, and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims

### Example 10: Identifying Causal Connectomic Abnormalities in PTSD Using spTMS/EEG Mapping

One way to address the limitations in using imaging to transition from a descriptive approach to mental illness to a circuit-based mechanistic one that could be used to directly guide much-needed novel interventions is to directly and non-invasively stimulate pre-defined cortical regions within the network using single pulses of transcranial magnetic stimulation (spTMS) while recording consequent brain activity, thereby establishing causality of signal flow. Using focal non-invasive transcranial magnetic brain stimulation (TMS) coupled with electroencephalography (EEG) a specific causal circuit dysfunction was identified that contributed to this phenotype's connectomic disturbances—implicating an excessive inhibitory rebound to stimulation in an executive control region of the right dorsolateral prefrontal cortex.

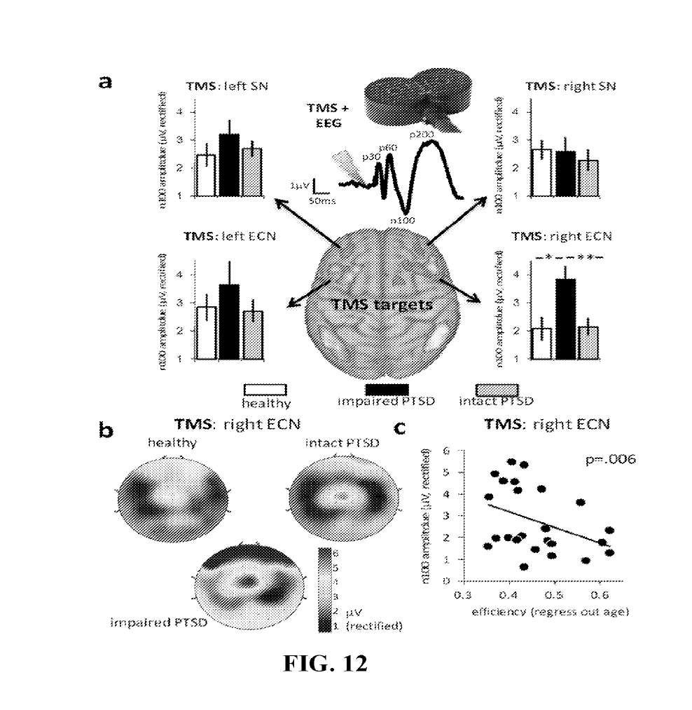

When measuring neural activity with electroencephalography (EEG), spTMS evokes a series of potentials (FIG. 12A inset). Early potentials (e.g. at 30 ms; p30) likely reflect evoked excitatory activity, while later potentials likely reflect a slow inhibitory rebound to stimulation unfolding over several hundred milliseconds ⁹²⁻⁹⁵. In humans, these later potentials (e.g. 60 ms, 100 ms, 200 ms; p60, n100, p200) appear distinct likely because they arise from different brain sources, and also have different pharmacological properties⁸¹. By stimulating various cortical regions with concurrent spTMS/EEG one can therefore reveal changes in causal signal flow, thereby mapping the “causal connectome.” Importantly, it is presently unknown how fMRI-measured connectivity relates to these components of causal signal flow evident during spTMS/EEG stimulation of network nodes.

In an attempt to decompose the network-level abnormalities in verbal memory impaired PTSD patients, spTMS/EEG connectomic mapping was applied to a subset of participants. It was examined whether causal abnormalities in evoked neural activity resulting from stimulation at several network regions were evident in memory-impaired PTSD participants, and whether these responses indexed their network efficiency as measured by resting fMRI. Stimulation was directed at four regions within the middle frontal gyrus bilaterally, with the posterior target being part of the executive control network (ECN) and the anterior target being part of the salience network (SN; FIG. 12A). These regions were chosen due to involvement of the middle frontal gyrus in memory ⁸²⁻⁸⁶, their membership in two different intrinsic networks ^{87,88}, and the prior concurrent spTMS/fMRI data demonstrating that stimulation to these regions evokes distinct patterns of network connectivity ⁸⁰. Targeting of these regions was also done in a manner consistent with how network regions of interest were defined for the resting fMRI analyses.

An omnibus repeated measures model including group, TMS site, potential and EEG electrode cluster revealed a TMS site by potential by group interaction, meaning that evoked activity differed across groups and stimulation sites (FIG. 12). Since TMS-evoked potentials differ with respect to biological mechanisms, then effects for each potential were separately examined. Significant TMS site by group interactions were observed for the p60 and n100 potentials. Examination of specific effects driving these interactions revealed most notably an increased n100 response to right ECN TMS stimulation in memory-impaired participants relative to both healthy and memory-intact PTSD participants (FIG. 12A). Right ECN TMS n100 differences were not moderated by electrode cluster, but were most evident in fronto-central electrodes (FIG. 12B). Resting fMRI-measured global efficiency was then correlated across PTSD participants with their right ECN TMS-evoked n100 responses, and found that those PTSD participants with the lowest resting fMRI efficiency had the largest n100 (FIG. 12C). Several other differences were found across stimulation sites and groups arising from group by TMS site by electrode cluster interactions for the p60 and n100 potentials, however none of these separated memory-impaired PTSD participants from both healthy and memory-intact PTSD participants and correlated with resting fMRI network efficiency (see supplemental results).

Prior work has shown a relationship between the magnitude of prefrontal TMS-evoked n100 potentials and the degree of neural inhibition⁸⁹. Thus, these findings suggest that impairments in information processing important for network efficiency may be at least in part mediated by excessive inhibition of signal flow through the right dorsolateral prefrontal node of the ECN—which could only be revealed through causal circuit interrogation with spTMS/EEG.

Methods

Anatomical targets for spTMS stimulation were determined based on an independent components analysis (ICA) of resting fMRI data from a separate group of individuals. The targets were placed on each participant's T1-weighted anatomical MRI for neuronavigation using the Visor2 LT 3D neuronavigation system (ANT Neuro, Netherlands). Each of four target sites were stimulated with 60 pulses (biphasic TMS pulses, 120% of resting motor threshold), interleaved at a random interval of 3±0.3 seconds using a MCF-B65 butterfly coil and a MagPro R30 TMS stimulator (MagVenture, Denmark). TMS sites included the ECN and SN nodes within the middle frontal gyrus bilaterally, as previously described⁸⁰. For the stimulation, the TMS coil was placed in a posterior to anterior direction, with an angle of 45 degrees to the nasion-inion axis. Participants were instructed to relax and to fixate at a cross located on the opposing wall during each stimulation.

64-channel EEG data were recorded using two 32-channel TMS-compatible BrainAmp DC amplifiers and the Easy EEG cap with extra flat, freely rotatable electrodes designed specifically for TMS applications (BrainProducts GmbH, Germany). Electrode impedances were kept below 5 kohms. EEG data were sampled at 5 kHz and an electrode attached to the tip of the nose was used as the reference. The electrodes were digitized relative to the scalp at the end of the spTMS-EEG session using the neuronavigation system. To avoid the artifact introduced by the coil recharge, the recharge time was delayed by 1500 ms.

The spTMS/EEG data were cleaned and analyzed using custom MATLAB (Mathworks, Natick, Mass.) scripts. The initial 10 ms data segment following TMS was discarded to remove the large stimulation-induced electric artifact. Non-physiological slow drifts in the EEG recordings were removed using a 0.01 Hz high-pass filter. The 60 Hz AC line noise artifact was identified via the Thompson F-statistic and removed by a multi-taper regression technique (https://www.nitrc.org/projects/cleanline/). The spectrally filtered EEG data were then epoched with respect to the TMS pulse (−500˜1500 ms), and re-referenced to the common average. Bad channels, trials, and remaining artifacts were subsequently removed using an in-house fully automated artifact rejection algorithm based on multiple runs of ICA. More specifically, the first ICA run rejected bad channels by quantifying and thresholding the surface Laplacian of each channel across independent components (ICs). The EEG signals of rejected channels were then interpolated from the adjacent channels using the spline interpolation, and the resulting EEG data were then re-referenced to the common average so as to prevent the reference from skewing towards the rejected bad channels. The second ICA run rejected bad trials by thresholding the magnitude of each trial across ICs. In the third ICA run, ICs related to the scalp muscle artifact, ocular artifact, ECG artifact, were rejected using a pattern classifier trained on expert-labeled ICs from another independent spTMS-EEG data sets.

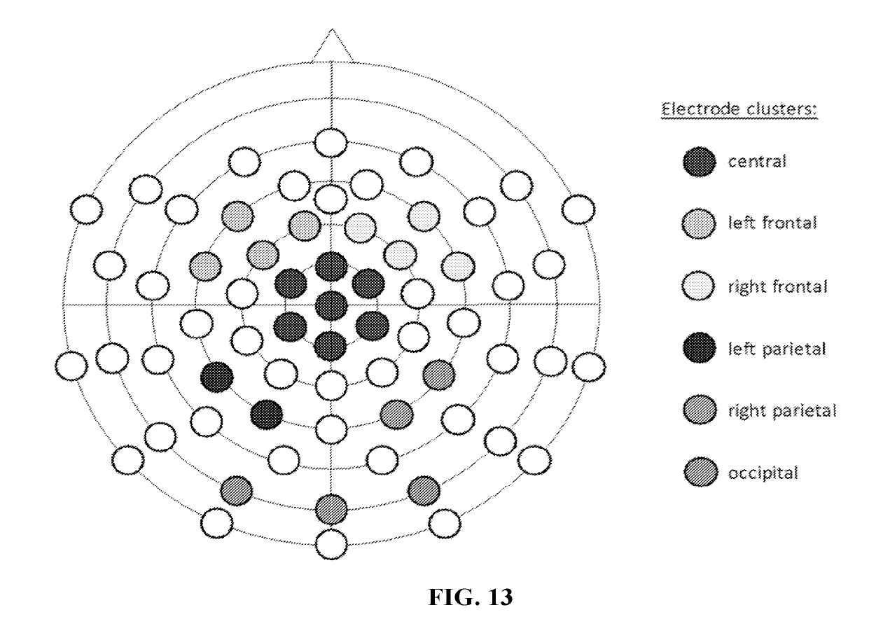

For each subject and stimulation site, the TMS-evoked potential was computed at each channel by averaging the clean EEG signal across trials. Average rectified potentials were quantified as the maximum response within the windows following time windows for each potential: p30 (25-35 ms), p60 (45-70 ms), n100 (100-150 ms), p200 (175-225 ms). Rectified magnitudes of each potential were entered into a repeated measures generalized estimating equation after averaging within each electrode clusters shown in FIG. 13 (electrode clusters were determined based on spatial proximity to and broad representation of the cortical network ROIs). Data from one intact memory PTSD participant were excluded from the analyses, as they were an outlier for three of the four TMS sites.

Results

TMS site by group by electrode cluster interactions were found for each of the four potentials analyzed (χ²'s>208, p's<0.001). Unlike for the n100 (FIG. 12), group differences were not found for the other potentials when collapsing across electrode clusters. Group by electrode cluster interactions were examined separately for each TMS site and potential to understand the factors driving the three-way interaction above. Significant interactions were seen for the n100 with left ECN TMS stimulation (χ²=23.3, p=0.01) and right SN stimulation (χ²=37.3, p<0.001), as well as for the p60 with left (χ²=18.7, p=0.044) and right (χ²=22.6, p=0.012) ECN TMS stimulation.

The n100 left ECN TMS group by electrode cluster interaction was driven by smaller n100's in both patient groups relative to healthy participants in left parietal electrodes (means: healthy (2.26), impaired (1.03), intact (1.17); post-hoc healthy vs. intact p=0.004, healthy vs. impaired p=0.013, intact vs. impaired p=0.713). The right SN TMS group by electrode cluster interaction was driven by significantly lower n100 responses to right SN TMS stimulation in parietal electrodes only in memory-impaired PTSD participants relative to either the healthy or memory-intact PTSD participants (means: healthy (1.8), impaired (0.39), intact (1.77); post-hoc impaired vs. healthy p<0.001, impaired vs. intact p<0.001, intact vs. healthy p=0.946). None of the effects described above correlated significantly with resting fMRI-determined network efficiency (χ²'s>1.57, p's>0.21).

The p60 left ECN TMS group by electrode cluster interaction was driven by smaller p60's in both patient groups relative to healthy participants in left frontal electrodes (means: healthy (2.32), impaired (0.77), intact (1.09); post-hoc healthy vs. intact p=0.026, healthy vs. impaired p=0.003, intact vs. impaired p=0.365). The p60 right ECN TMS group by electrode cluster interaction was driven by smaller p60's only in intact-memory PTSD participants relative to healthy and impaired-memory PTSD participants in right frontal electrodes (means: healthy (2.11), impaired (2.01), intact (1.09); post-hoc healthy vs. intact p=0.042, healthy vs. impaired p=0.858, intact vs. impaired p=0.018). None of the effects described above correlated significantly with resting fMRI-determined network efficiency (χ²'s>1.8, p's>0.18).

Moreover, direct circuit interrogation with concurrent TMS/EEG identified a prefrontal ECN network node wherein stimulation causally evokes abnormal fronto-central neural responses that likely signify an excessive inhibitory response to stimulation (i.e. a stronger rebound from network perturbation). This abnormality was evident only in network/memory-impaired PTSD participants and in a manner related to individual differences in resting fMRI-measured network efficiency. Using spTMS/fMRI in healthy individuals, it was previously found that stimulation to this right ECN node resulted in stronger ECN-default mode interactions than did stimulation to the right SN node ⁸⁰, consistent with its implication in network efficiency here.

Intriguingly, the vast majority of repetitive TMS treatment studies for PTSD have targeted a right-sided prefrontal region in the vicinity of the ECN node ^{90, 91}, demonstrating efficacy for both low and high frequency stimulation as one-size-fits-all interventions. However, since these studies examined only clinical outcome, the mechanism of action for the treatment remains unknown, as does who responds best and why (which may differ between treatment protocols). The results therefore provide a platform to transition from correlation-based neuroimaging to a causal-connectomic approach that identifies a putative target for repetitive TMS treatment (right executive network dorsolateral prefrontal cortex) and a physiological signal to track for normalization (100 ms TMS-induced potential) in a specifically-defined form of PTSD. It is anticipated that the optimal TMS treatment protocol would differ between those with and without the network/memory phenotype, but that the protocol could be guided by knowledge of the physiological target of treatment at an individual patient level. More generally, the spTMS/EEG results also provide a perspective on which elements of causal signal flow within a network relate to fMRI-measured network connectivity, and establish TMS as a causal mapping tool for understanding the neural basis of these common network measures.

### Example 11: Establishing the Impact of the Network/Memory Phenotype on Treatment Outcome

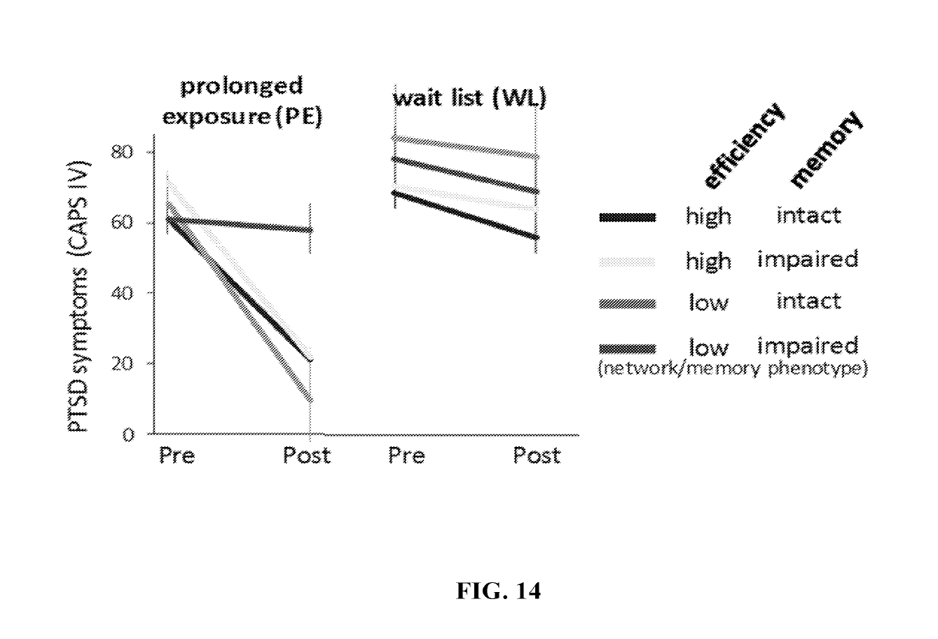

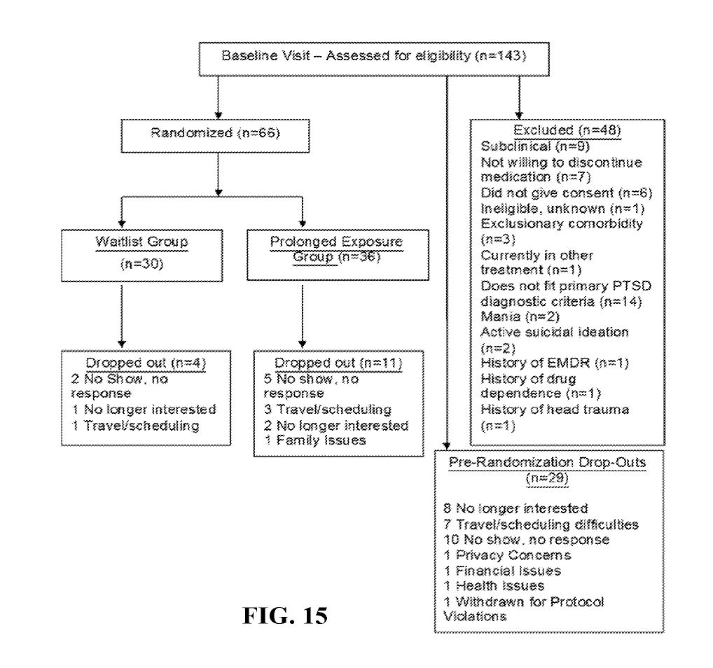

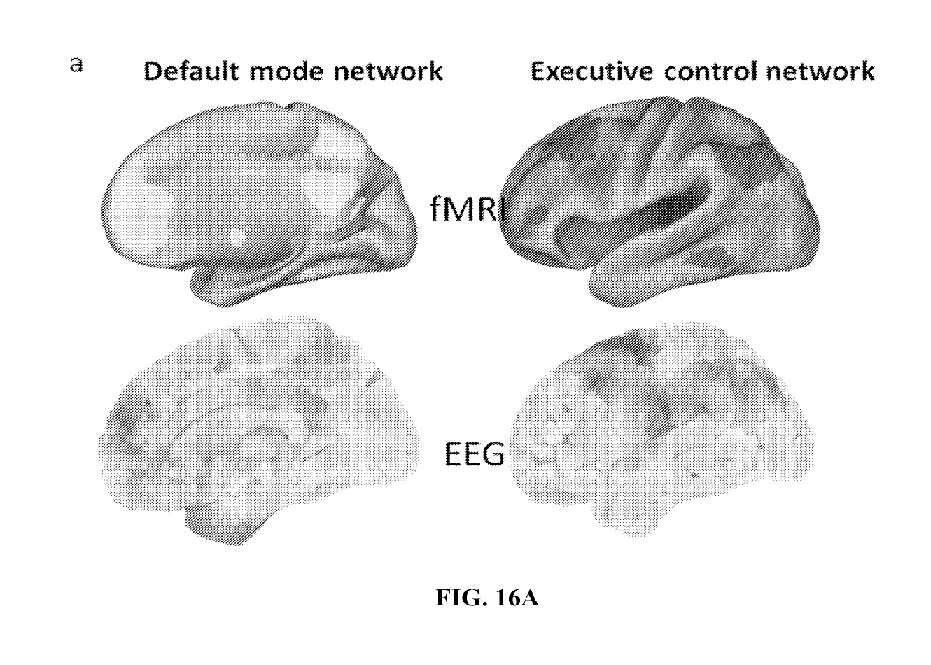

To test the clinical relevance of the identified impairments in network efficiency and memory for PTSD, it was examined how these impairments influenced response to treatment. Trauma-focused psychotherapy, such as prolonged exposure, is considered the gold-standard treatment for PTSD (better than medications) and centrally involves learning and memory ^{21,25}. PTSD patients (n=66, largely overlapping with study 1) were randomized to receive either prolonged exposure psychotherapy or a wait list minimal attention control (see FIG. 15 and table 4). An intent-to-treat analysis (i.e. including all participants as they were randomized in a mixed model, including drop-outs) revealed a robust group by time interaction on clinician-assessed PTSD symptoms, and pre-specified primary outcome (FIG. 14). This interaction was driven, as expected, by greater improvement in symptoms with exposure therapy than the wait-list arm.

It was next examined whether impairments in memory and global efficiency moderated the effect of treatment (compared to wait-list), such that a difference in outcome across arms is seen in relation to the network/memory phenotype delineated above. Including both memory and efficiency in the mixed model, a highly significant interaction was found between network efficiency and memory with group and time (FIG. 14). Patients with both impaired verbal memory and reduced global efficiency (i.e. those with the network/memory phenotype) saw no improvement in symptoms after treatment—similar to the wait-list control. By contrast, patients with intact memory and/or efficiency frequently remitted with therapy (but did not with wait-list; remission is CAPS-IV≤20). Use of the binary network/memory-impairment phenotype biomarker resulted in similarly robust moderation effects (FIG. 14). Importantly, neither verbal memory nor network efficiency alone moderated outcome (FIG. 14). Thus, the network/memory phenotype, despite being equally symptomatic as PTSD patients without this phenotype, was nonetheless associated with profound treatment resistance.

### Example 12: Measurement of Spatial Connectivity Maps

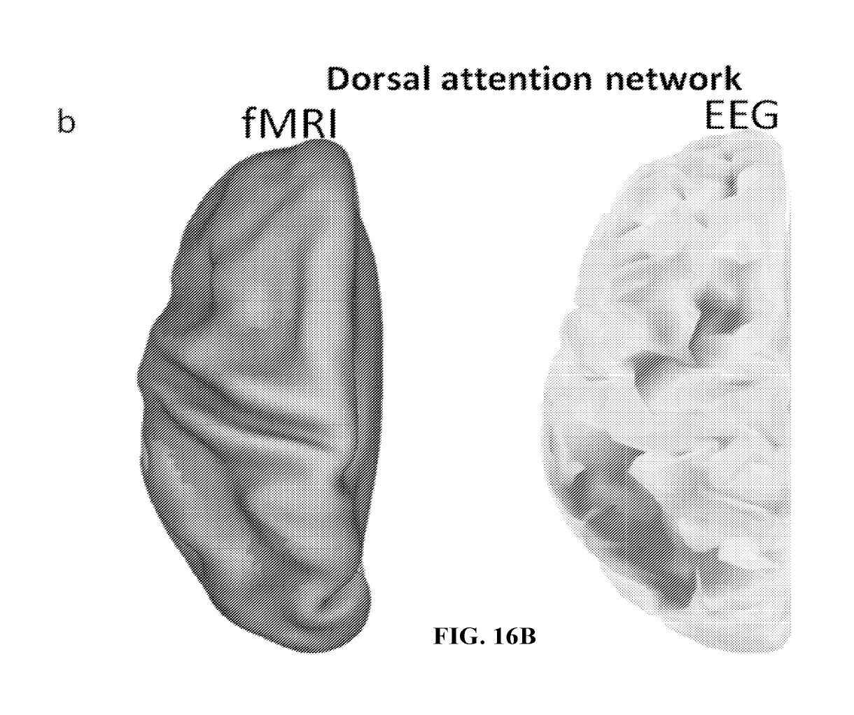

FIGS. 16A and 16B demonstrate implementation of one method for assessing EEG connectivity. Of note, this method used in these figures yields spatial connectivity maps that closely resemble fMRI connectivity maps, demonstrating the utility of EEG for assessing fMRI cognitive network connectivity. This method is based on a report using MEG by Hipp et al. (Hipp J F, Hawellek D J, Corbetta M, Siegel M, Engel A K. “Large-scale cortical correlation structure of spontaneous oscillatory activity”, Nature Neuroscience, 15 (6): 884-890; 2013). Specifically, at one frequency (here 10 Hz was used), the ongoing amplitude of the instantaneous power of the EEG signal at this frequency is calculated (termed the power envelope at the 10 Hz carrier frequency). The time course of the power envelope signal for two or more brain regions then serves as the basis on which correlations are calculated (indicating EEG connectivity). As demonstrated by the figures, seeding the time course of specific example cognitive network regions and examining power envelope correlations across the brain yields patterns of connectivity that closely resemble those seen when similarly seeding connectivity in an fMRI scan. Hipp and colleagues have also shown that in MEG this method yields connectivity estimates that correlate with the same individual's fMRI connectivity (Hipp J F, Siegel M. “BOLD fMRI Correlation Reflects Frequency-Specific Neuronal Correlation”, Current Biology 25: 1368-1374; 2015).

### CERTAIN EMBODIMENTS

### Embodiment 1

A method of treating post-traumatic stress disorder in a subject in need thereof, said method comprising: (i) determining connectivity between a first cognitive region within the brain of said subject and a second cognitive region within the brain of said subject or determining a complex cognitive behavioral deficiency in said subject; and (ii) administering a post-traumatic stress disorder treatment to said subject.

### Embodiment 2

The method of Embodiment 1, wherein said first cognitive region and said second cognitive region are independently selected from the group consisting of left middle frontal gyms, left inferior frontal gyrus, left inferior parietal lobule, left middle temporal gyrus, left thalamus, right middle frontal gyrus, right inferior frontal gyrus, right inferior parietal lobule, right dorsomedial PFC, left lateral cerebellum, right caudate, left anterior middle frontal gyms, left insula, dorsal anterior cingulate cortex (ACC), right anterior middle frontal gyrus, right insula, left lateral cerebellum, right lateral cerebellum, left frontal eye fields, left intraparietal sulcus, left inferior frontal cortex, left inferior temporal gyrus, right frontal eye fields, right intraparietal sulcus, right inferior frontal cortex, right inferior temporal gyrus, right lateral cerebellum, medial prefrontal cortex, left angular gyrus, right superior frontal gyms, posterior cingulate gyrus, mid-cingulate gyrus, right angular gyrus, thalamus, left hippocampus, and right hippocampus; wherein said first cognitive region is different from said second cognitive region.

### Embodiment 3

The method of one of Embodiments 1 to 2, wherein said determining connectivity is performed using a functional connectivity analysis.

### Embodiment 4

The method of Embodiment 3, wherein said functional connectivity analysis is a blood flow analysis.

### Embodiment 5

The method of Embodiment 4, wherein said blood flow analysis is an fMRI analysis.

### Embodiment 6

The method of Embodiment 4, wherein said blood flow analysis is a near infrared spectroscopy (NIRS) analysis.

### Embodiment 7

The method of Embodiment 3, wherein said functional connectivity analysis is an electroencephalogram (EEG) analysis.

### Embodiment 8

The method of Embodiment 3, wherein said functional connectivity analysis is a magnetoencephalography (MEG) analysis.

### Embodiment 9

The method of any one of Embodiments 1-9, wherein said determining connectivity comprises administering a transcranial magnetic stimulation (TMS) thereby producing an evoked response.

### Embodiment 10

The method of Embodiment 9, wherein said TMS is administered to the right executive control network (ECN).

### Embodiment 11

The method of any one of Embodiments 9-10, wherein said evoked response is monitored by Electroencephalography (EEG).

### Embodiment 12

The method of Embodiment 12, wherein an amplitude of the evoked response is measured by EEG at about 30-250 ms after stimulation.

### Embodiment 13

The method of Embodiment 1 or 2, wherein said determining connectivity is performed using a structural connectivity analysis.

### Embodiment 14

The method of Embodiment 13, wherein said structural connectivity analysis is a diffusion-weighted structural connectivity analysis.

### Embodiment 15

The method of one of Embodiments 1 to 14 comprising determining a complex cognitive behavioral deficiency in said subject.

### Embodiment 16

The method of Embodiment 15, wherein said complex cognitive behavioral deficiency is a memory deficiency.

### Embodiment 17

The method of Embodiment 9, wherein said memory deficiency is a long term memory deficiency, a working memory deficiency, a short term memory deficiency, a delayed recall deficiency or an immediate recall deficiency.

### Embodiment 18

The method of one of Embodiments 15 to 17, wherein said determining is performed using a card sorting analysis, reward or punishment learning tests, planning test, or navigation test.

### Embodiment 19

The method of one of Embodiments 1 to 18, wherein said post-traumatic stress disorder treatment comprises psychotherapy.

### Embodiment 20

The method of any one of Embodiments 1 to 18 wherein said treatment comprises repetitive transcranial magnetic stimulation (rTMS).

### Embodiment 21

The method of Embodiment 20, wherein said rTMS is administered to the right executive control network (ECN).

### Embodiment 22

The method of Embodiment 19, wherein said psychotherapy is selected from the group consisting of prolonged exposure therapy, cognitive processing therapy, cognitive behavioral therapy, eye movement and desensitization therapy, acceptance and commitment therapy, and interpersonal psychotherapy.

### Embodiment 23

A method of determining connectivity between cognitive regions in a patient suffering from or suspected of suffering from a post-traumatic stress disorder, the method comprising determining connectivity between a first cognitive region within the brain of said subject and a second cognitive region within the brain of said subject or determining a complex cognitive behavioral deficiency in said subject.

### Embodiment 24

The method of Embodiment 23, wherein said patient is undergoing a course of treatment for a post-traumatic stress disorder.

### Embodiment 25

A system comprising: at least one processor; and at least one memory including program code which when executed by the at least one memory provides operations comprising: determining a connectivity between a first cognitive region and a second cognitive region within a brain of a subject and/or determining a complex cognitive behavioral deficiency in said subject; and providing, via a user interface, a post-traumatic stress disorder treatment plan for said subject.

### Embodiment 26

The system of Embodiment 26, wherein the connectivity between the first cognitive region and the second cognitive region within the brain of the subject and the complex cognitive behavioral deficiency in the subject comprises a biomarker associated with the subject.

### Embodiment 27

The system of Embodiment 27, further comprising determining a presence of the biomarker in the subject by at least: screening the subject for memory deficit; and performing, on the brain of the subject, an imaging test, when a result of the screening indicates that the subject exhibits memory deficit.

### Embodiment 28

The system of Embodiment 27, wherein the imaging test comprises functional magnetic resonance imaging (fMRI) and/or magnetoencephalography (MEG) or electroencephalography (EEG).

### Embodiment 29

The system of any one of Embodiments 27-28, further comprising determining, based on a presence of the biomarker in the subject, the post-traumatic stress disorder treatment plan for the subject.

### Embodiment 30

The system of Embodiment 29, wherein psychotherapy is excluded from the post-traumatic stress disorder treatment plan for the subject, when the biomarker is determined to be present in the subject.

### Embodiment 31

The system of Embodiment 29, wherein psychotherapy is excluded from the post-traumatic stress disorder treatment plan for the subject, when the subject exhibits abnormal connectivity and complex cognitive behavioral deficiency.

### Embodiment 32

The system of Embodiment 29, wherein psychotherapy is included in the post-traumatic stress disorder treatment plan for the subject, when the biomarker is determined to be not present in the subject.

### Embodiment 33

The system of Embodiment 29, wherein non-invasive brain stimulation is included the post-traumatic stress disorder treatment plan for the subject, when the biomarker is determined to be present in the subject.

### Embodiment 34

The system of Embodiments 30-32, wherein the treatment plan further includes one or more of psychotherapy, medication and non-invasive brain stimulation.

### Embodiment 35

The system of Embodiment 34, wherein the non-invasive brain stimulation is administered based at least in part on the connectivity between the first cognitive region and the second cognitive region within the brain of said subject.

### Embodiment 36

The system of Embodiment 25, wherein determining the connectivity between the first cognitive region and the second cognitive region within the brain of the subject comprises evoking a response by administering transcranial magnetic stimulation (TMS).

### Embodiment 37

The system of Embodiment 36, wherein the TMS is administered to a right executive control network (ECN).

### Embodiment 38

The system of Embodiments 36-37, further comprising monitoring, via Electroencephalography (EEG), the response evoked by the administration of the TMS.

### Embodiment 39

The system of Embodiment 36, wherein the monitoring of the response evoked by the administration of the TMS comprises measuring, by EEG, an amplitude of the response approximately 30-250 milliseconds (ms) subsequent to the administration of TMS.

### Embodiment 40

The system of Embodiment 25, wherein the system is further configured to perform operations comprising the method as recited in any of Embodiments 2-17.

### Embodiment 41

A non-transitory computer-readable storage medium including program code which when executed by at least one processor causes operations comprising: determining a connectivity between a first cognitive region and a second cognitive region within a brain of a subject and/or determining a complex cognitive behavioral deficiency in said subject; and providing, via a user interface, a post-traumatic stress disorder treatment plan for said subject.

### Embodiment 42

The computer-readable storage medium of Embodiment 41, wherein the connectivity between the first cognitive region and the second cognitive region within the brain of the subject and the complex cognitive behavioral deficiency in the subject comprises a biomarker associated with the subject.

### Embodiment 43

The computer-readable storage medium of Embodiment 42, further comprising determining a presence of the biomarker in the subject by at least: screening the subject for memory deficit; and performing, on the brain of the subject, an imaging test, when a result of the screening indicates that the subject exhibits memory deficit.

### Embodiment 44

The computer-readable storage medium of Embodiment 41, wherein the imaging test comprises functional magnetic resonance imaging (fMRI) and/or magnetoencephalography (MEG) or electroencephalography (EEG).

### Embodiment 45

The computer-readable storage medium of Embodiment 41, further comprising determining, based on a presence of the biomarker in the subject, the post-traumatic stress disorder treatment plan for the subject.

### Embodiment 46

The computer-readable storage medium of Embodiment 43, wherein psychotherapy is excluded from the post-traumatic stress disorder treatment plan for the subject, when the biomarker is determined to be present in the subject.

### Embodiment 47

The computer-readable storage medium of Embodiment 43, wherein psychotherapy is excluded from the post-traumatic stress disorder treatment plan for the subject, when the subject exhibits abnormal connectivity and complex cognitive behavioral deficiency.

### Embodiment 48

The computer-readable storage medium of Embodiment 43, wherein psychotherapy is included in the post-traumatic stress disorder treatment plan for the subject, when the biomarker is determined to be not present in the subject.

### Embodiment 49

The computer-readable storage medium of Embodiments 44-45, wherein the treatment plan further includes one or more of psychotherapy, medication and non-invasive brain stimulation.

### Embodiment 50

The computer-readable storage medium of Embodiment 46, wherein the non-invasive brain stimulation is administered based at least in part on the connectivity between the first cognitive region and the second cognitive region within the brain of said subject.

### Embodiment 51

The computer-readable storage medium of Embodiment 39, wherein determining the connectivity between the first cognitive region and the second cognitive region within the brain of the subject comprises evoking a response by administering transcranial magnetic stimulation (TMS).

### Embodiment 52

The computer-readable storage medium of Embodiment 49, wherein the TMS is administered to a right executive control network (ECN).

### Embodiment 53

The computer-readable storage medium of Embodiments 49-50, further comprising monitoring, via Electroencephalography (EEG), the response evoked by the administration of the TMS.

### Embodiment 54

The computer-readable storage medium of Embodiment 51, wherein the monitoring of the response evoked by the administration of the TMS comprises measuring, by EEG, an amplitude of the response approximately 30-250 milliseconds (ms) subsequent to the administration of TMS.

### Embodiment 55

The computer-readable storage medium of Embodiment 41, wherein the operations further comprise the method as recited in any of Embodiments 1-22.

### Embodiment 56

An apparatus comprising: means for determining a connectivity between a first cognitive region and a second cognitive region within a brain of a subject and/or means for determining a complex cognitive behavioral deficiency in said subject; and means for providing a post-traumatic stress disorder treatment plan for said subject.

### Embodiment 57

The apparatus of Embodiment 56, wherein the connectivity between the first cognitive region and the second cognitive region within the brain of the subject and the complex cognitive behavioral deficiency in the subject comprises a biomarker associated with the subject.

### Embodiment 58

The apparatus of Embodiment 57, wherein the apparatus is further configured to determine a presence of the biomarker in the subject by at least: screening the subject for memory deficit; and performing, on the brain of the subject, an imaging test, when a result of the screening indicates that the subject exhibits memory deficit.

### Embodiment 59

The apparatus of Embodiment 58, wherein the imaging test comprises functional magnetic resonance imaging (fMRI) and/or magnetoencephalography (MEG) or electroencephalography (EEG).

### Embodiment 60

The apparatus of Embodiment 58, wherein the apparatus is further configured to determine, based on a presence of the biomarker in the subject, the post-traumatic stress disorder treatment plan for the subject.

### Embodiment 61

The apparatus of Embodiment 60, wherein the apparatus is configured to exclude psychotherapy from the post-traumatic stress disorder treatment plan for the subject, when the biomarker is determined to be present in the subject.

### Embodiment 62

The apparatus of Embodiment 60, wherein the apparatus is configured to exclude psychotherapy from the post-traumatic stress disorder treatment plan for the subject, when the subject exhibits abnormal connectivity and complex cognitive behavioral deficiency.

### Embodiment 63

The apparatus of Embodiment 60, wherein the apparatus is configured to include psychotherapy in the post-traumatic stress disorder treatment plan for the subject, when the biomarker is determined to be not present in the subject.

### Embodiment 64

The apparatus of Embodiments 59-60, wherein the treatment plan further includes one or more of psychotherapy, medication and non-invasive brain stimulation.

### Embodiment 65

The apparatus of Embodiment 61, wherein the non-invasive brain stimulation is administered based at least in part on the connectivity between the first cognitive region and the second cognitive region within the brain of said subject.

### Embodiment 66

The apparatus of Embodiment 56, wherein the apparatus is configured to determine the connectivity between the first cognitive region and the second cognitive region within the brain of the subject by at least evoking a response by administering transcranial magnetic stimulation (TMS).

### Embodiment 67

The apparatus of Embodiment 69, wherein the TMS is administered to a right executive control network (ECN).

### Embodiment 68

The apparatus of Embodiments 66-67, wherein the apparatus is further configured to monitor, via Electroencephalography (EEG), the response evoked by the administration of the TMS.

### Embodiment 69

The apparatus of Embodiment 68, wherein the apparatus is configured to monitor the response evoked by the administration of the TMS by at least measuring, by EEG, an amplitude of the response approximately 30-250 milliseconds (ms) subsequent to the administration of TMS.

### Embodiment 70

The apparatus of Embodiment 56, further comprising means for performing the method as recited in any of Embodiments 1-22.

### Embodiment 71

A method of treating post-traumatic stress disorder in a subject in need thereof, said method comprising: determining a connectivity between a first cognitive region within a brain of said subject and a second cognitive region within the brain of said subject and/or determining a complex cognitive behavioral deficiency in said subject; and providing, via a user interface, a post-traumatic stress disorder treatment plan for said subject.

### Embodiment 72

The method of Embodiment 71, wherein the connectivity between the first cognitive region and the second cognitive region within the brain of the subject and the complex cognitive behavioral deficiency in the subject comprises a biomarker associated with the subject.

### Embodiment 73

The method of Embodiment 72, further comprising determining a presence of the biomarker in the subject by at least: screening the subject for memory deficit; and performing, on the brain of the subject, an imaging test, when a result of the screening indicates that the subject exhibits memory deficit.

### Embodiment 74

The method of Embodiment 72, wherein the imaging test comprises functional magnetic resonance imaging (fMRI) and/or magnetoencephalography (MEG).

### Embodiment 75

The method of Embodiment 72, further comprising determining, based on a presence of the biomarker in the subject, the post-traumatic stress disorder treatment plan for the subject.

### Embodiment 76

The method of Embodiment 75, wherein psychotherapy is excluded from the post-traumatic stress disorder treatment plan for the subject, when the biomarker is determined to be present in the subject.

### Embodiment 77

The method of Embodiment 75, wherein psychotherapy is excluded from the post-traumatic stress disorder treatment plan for the subject, when the subject exhibits abnormal connectivity and complex cognitive behavioral deficiency.

### Embodiment 78

The method of Embodiment 75, wherein psychotherapy is included in the post-traumatic stress disorder treatment plan for the subject, when the biomarker is determined to be not present in the subject.

### Embodiment 79

The method of Embodiments 76-78, wherein the treatment plan further includes one or more of psychotherapy, medication and non-invasive brain stimulation.

### Embodiment 80

The method of Embodiment 79, wherein the non-invasive brain stimulation is administered based at least in part on the connectivity between the first cognitive region and the second cognitive region within the brain of said subject.

### Embodiment 81

The method of Embodiment 71, wherein determining the connectivity between the first cognitive region and the second cognitive region within the brain of the subject comprises evoking a response by administering transcranial magnetic stimulation (TMS).

### Embodiment 82

The method of Embodiment 81, wherein the TMS is administered to a right executive control network (ECN).

### Embodiment 83

The method of Embodiments 81-82, further comprising monitoring, via Electroencephalography (EEG), the response evoked by the administration of the TMS.

### Embodiment 84

The method of Embodiment 83, wherein the monitoring of the response evoked by the administration of the TMS comprises measuring, by EEG, an amplitude of the response approximately 30-250 milliseconds (ms) subsequent to the administration of TMS.

### Embodiment 85

The method of Embodiment 71, further comprising the method as recited in any of Embodiments 1-22.

### Embodiment 86

A method of treating a subject having or suspected of having inhibitory right ECN post-traumatic stress disorder, the method comprising administering TMS.

### Embodiment 87

The method of Embodiment 86, wherein TMS is administered to the right ECN.

### Embodiment 88

The method of any of Embodiments 86-87, wherein TMS is delivered repetitively in a pattern that is intended to induce plasticity.

### Embodiment 89

The method of any of Embodiments 86-87, wherein TMS is rTMS.

### Embodiment 90

The method of Embodiment 89, wherein rTMS comprises stimulation at greater than 5 Hz.

### Embodiment 91

The method of Embodiment 89, wherein rTMS comprises stimulation at less than or equal to 1 Hz.

### Embodiment 92

The method of Embodiment 89, wherein rTMS comprises either a continuous or an intermittent theta burst pattern.

### REFERENCES

1. Etkin, A., Gyurak, A. & O'Hara, R. A neurobiological approach to the cognitive deficits of psychiatric disorders. Dialogues Clin Neurosci 15, 419-429 (2013).
2. Scott, J. C., et al. A quantitative meta-analysis of neurocognitive functioning in posttraumatic stress disorder. Psychological bulletin 141, 105-140 (2015).
3. Sheffield, J. M., et al. Fronto-parietal and cingulo-opercular network integrity and cognition in health and schizophrenia. Neuropsychologia 73, 82-93 (2015).
4. Chen, A. C. & Etkin, A. Hippocampal network connectivity and activation differentiates post-traumatic stress disorder from generalized anxiety disorder. Neuropsychopharmacology 38, 1889-1898 (2013).
5. Zhang, Y., et al. Intranetwork and internetwork functional connectivity alterations in post-traumatic stress disorder. J Affect Disord 187, 114-121 (2015).
6. Latora, V. & Marchiori, M. Efficient behavior of small-world networks. Phys Rev Lett 87, 198701 (2001).
7. Bullmore, E. & Sporns, O. The economy of brain network organization. Nature reviews. Neuroscience 13, 336-349 (2012).
8. Sporns, O. Network attributes for segregation and integration in the human brain. Current opinion in neurobiology 23, 162-171 (2013).
9. Chan, M. Y., Park, D. C., Savalia, N. K., Petersen, S. E. & Wig, G. S. Decreased segregation of brain systems across the healthy adult lifespan. Proc Natl Acad Sci USA 111, E4997-5006 (2014).
10. Smith, S. M., et al. Correspondence of the brain's functional architecture during activation and rest. Proc Natl Acad Sci USA 106, 13040-13045 (2009).
11. Laird, A. R., et al. Behavioral interpretations of intrinsic connectivity networks. J Cogn Neurosci 23, 4022-4037 (2011).
12. Shirer, W. R., Ryali, S., Rykhlevskaia, E., Menon, V. & Greicius, M. D. Decoding subject-driven cognitive states with whole-brain connectivity patterns. Cereb Cortex 22, 158-165 (2012).
13. Richiardi, J., et al. BRAIN NETWORKS. Correlated gene expression supports synchronous activity in brain networks. Science 348, 1241-1244 (2015).
14. Rubinov, M., Ypma, R. J., Watson, C. & Bullmore, E. T. Wiring cost and topological participation of the mouse brain connectome. Proc Natl Acad Sci USA 112, 10032-10037 (2015).
15. Nievergelt, C. M., et al. Genomic predictors of combat stress vulnerability and resilience in U.S. Marines: A genome-wide association study across multiple ancestries implicates PRTFDC1 as a potential PTSD gene. Psychoneuroendocrinology 51, 459-471 (2015).
16. APA. Diagnostic and statistical manual of mental disorders, (American Psychiatric Press, Washington D.C., 1994).
17. APA. Diagnostic and statistical manual of mental disorders, (American Psychiatric Press, Washington D.C., 2013).
18. Galatzer-Levy, I. R. & Bryant, R. A. 636,120 Ways to Have Posttraumatic Stress Disorder. Perspectives on psychological science: a journal of the Association for Psychological Science 8, 651-662 (2013).
19. Hoge, C. W., Riviere, L. A., Wilk, J. E., Herrell, R. K. & Weathers, F. W. The prevalence of post-traumatic stress disorder (PTSD) in US combat soldiers: a head-to-head comparison of DSM-5 versus DSM-IV-TR symptom criteria with the PTSD checklist. The lancet. Psychiatry 1, 269-277 (2014).
20. Insel, T. R. & Cuthbert, B. N. Endophenotypes: bridging genomic complexity and disorder heterogeneity. Biol Psychiatry 66, 988-989 (2009).
21. IOM. Treatment of posttraumatic stress disorder: An assessment of the evidence, (The National Academies Press, Washington, D C, 2008).
22. Garrison, K. A., Scheinost, D., Finn, E. S., Shen, X. & Constable, R. T. The (in)stability of functional brain network measures across thresholds. Neuroimage 118, 651-661 (2015).
23. Kitzbichler, M. G., Henson, R. N., Smith, M. L., Nathan, P. J. & Bullmore, E. T. Cognitive effort drives workspace configuration of human brain functional networks. J Neurosci 31, 8259-8270 (2011).
24. van den Heuvel, M. P., Stam, C. J., Kahn, R. S. & HulshoffPol, H. E. Efficiency of functional brain networks and intellectual performance. J Neurosci 29, 7619-7624 (2009).
25. Rothbaum, B. O. & Davis, M. Applying learning principles to the treatment of post-trauma reactions. Annals of the New York Academy of Sciences 1008, 112-121 (2003).
26. Hawrylycz, M. J., et al. An anatomically comprehensive atlas of the adult human brain transcriptome. Nature 489, 391-399 (2012).
27. Fuge, P., et al. Interaction of early life stress and corticotropin-releasing hormone receptor gene: effects on working memory. Biol Psychiatry 76, 888-894 (2014).
28. Tyrka, A. R., et al. Interaction of childhood maltreatment with the corticotropin-releasing hormone receptor gene: effects on hypothalamic-pituitary-adrenal axis reactivity. Biol Psychiatry 66, 681-685 (2009).
29. Hsu, D. T., et al. Variation in the corticotropin-releasing hormone receptor 1 (CRHR1) gene influences fMRI signal responses during emotional stimulus processing. J Neurosci 32, 3253-3260 (2012).
30. Ehlis, A. C., et al. Influence of a genetic variant of the neuronal growth associated protein Stathmin 1 on cognitive and affective control processes: an event-related potential study. Am J Med Genet B Neuropsychiatr Genet 156B, 291-302 (2011).
31. Stjepanovic, D., Lorenzetti, V., Yucel, M., Hawi, Z. & Bellgrove, M. A. Human amygdala volume is predicted by common DNA variation in the stathmin and serotonin transporter genes. Transl Psychiatry 3, e283 (2013).
32. Davidson, J. R. Remission in post-traumatic stress disorder (PTSD): effects of sertraline as assessed by the Davidson Trauma Scale, Clinical Global Impressions and the Clinician-Administered PTSD scale. International clinical psychopharmacology 19, 85-87 (2004).
33. Caeyenberghs, K., et al. Altered structural networks and executive deficits in traumatic brain injury patients. Brain structure & function 219, 193-209 (2014).
34. Crossley, N. A., et al. Cognitive relevance of the community structure of the human brain functional coactivation network. Proc Natl Acad Sci USA 110, 11583-11588 (2013).
35. Santarnecchi, E., Galli, G., Polizzotto, N. R., Rossi, A. & Rossi, S. Efficiency of weak brain connections support general cognitive functioning. Hum Brain Mapp 35, 4566-4582 (2014).
36. Spielberg, J. M., McGlinchey, R. E., Milberg, W. P. & Salat, D. H. Brain Network Disturbance Related to Posttraumatic Stress and Traumatic Brain Injury in Veterans. Biol Psychiatry (2015).
37. Lei, D., et al. Disrupted Functional Brain Connectome in Patients with Posttraumatic Stress Disorder. Radiology, 141700 (2015).
38. Wang, X. D., et al. Nectin-3 links CRHR1 signaling to stress-induced memory deficits and spine loss. Nat Neurosci 16, 706-713 (2013).
39. Ivy, A. S., et al. Hippocampal dysfunction and cognitive impairments provoked by chronic early-life stress involve excessive activation of CRH receptors. J Neurosci 30, 13005-13015 (2010).
40. Refojo, D., et al. Glutamatergic and dopaminergic neurons mediate anxiogenic and anxiolytic effects of CRHR1. Science 333, 1903-1907 (2011).
41. Sheng, H., et al. Corticotropin-releasing hormone (CRH) depresses n-methyl-D-aspartate receptor-mediated current in cultured rat hippocampal neurons via CRH receptor type 1. Endocrinology 149, 1389-1398 (2008).
42. Dunlop, B. W., et al. Evaluation of a corticotropin releasing hormone type 1 receptor antagonist in women with posttraumatic stress disorder: study protocol for a randomized controlled trial. Trials 15, 240 (2014).
43. Plotsky, P. M., et al. Long-term consequences of neonatal rearing on central corticotropin-releasing factor systems in adult male rat offspring. Neuropsychopharmacology 30, 2192-2204 (2005).
44. Mallei, A., et al. Synaptoproteomics of learned helpless rats involve energy metabolism and cellular remodeling pathways in depressive-like behavior and antidepressant response. Neuropharmacology 60, 1243-1253 (2011).
45. Martel, G., et al. Murine GRPR and stathmin control in opposite directions both cued fear extinction and neural activities of the amygdala and prefrontal cortex. PLoS One 7, e30942 (2012).
46. Chauvin, S. & Sobel, A. Neuronal stathmins: a family of phosphoproteins cooperating for neuronal development, plasticity and regeneration. Prog Neurobiol 126, 1-18 (2015).
47. Insel, T. R. The NIMH Research Domain Criteria (RDoC) Project: precision medicine for psychiatry. Am J Psychiatry 171, 395-397 (2014).
48. Chan, M. Y., Park, D. C., Savalia, N. K., Petersen, S. E. & Wig, G. S. Decreased segregation of brain systems across the healthy adult lifespan. Proc Natl Acad Sci USA 111, E4997-5006 (2014).
49. Blake, D. D., et al. The development of a Clinician-Administered PTSD Scale. J Trauma Stress 8, 75-90 (1995).
50. First, M. B. A practical prototypic system for psychiatric diagnosis: the ICD-11 Clinical Descriptions and Diagnostic Guidelines. World Psychiatry 11, 24-25 (2012).
51. Wechsler, D. Wechsler Abbreviated Scale of Intelligence. (The Psychological Corporation: Harcourt Brace & Company, New York, N.Y., 1999).
52. APA. Diagnostic and statistical manual of mental disorders, (American Psychiatric Press, Washington D.C., 2013).
54. APA. Diagnostic and statistical manual of mental disorders, (American Psychiatric Press, Washington D.C., 1994).
55. Corrigan, J. D. & Bogner, J. Initial reliability and validity of the Ohio State University TBI Identification Method. The Journal of head trauma rehabilitation 22, 318-329 (2007).
56. Skevington, S. M., Lotfy, M., O'Connell, K. A. & Group, W. The World Health Organization's WHOQOL-BREF quality of life assessment: psychometric properties and results of the international field trial. A report from the WHOQOL group. Quality of life research: an international journal of quality of life aspects of treatment, care and rehabilitation 13, 299-310 (2004).
57. Babor, T. F., de la Fuente, J. R., Saunders, J. & Grant, M. AUDIT: The Alcohol Use Disorders Identification Test. Guidelines for use in primary health care, (World Health Organization, Geneva, Switzerland, 1992).
58. Weathers, F., Litz, B., Herman, D., Huska, J. & Keane, T. The PTSD Checklist (PCL): reliability, validity and diagnostic utility. in The International Society For Traumatic Stress Studies (San Antonio, Tex., 1993).
59. Beck, A. T., Steer, R. A. & Brown, G. K. Manual for Beck Depression Inventory-II, (Psychological Corporation, San Antonio, Tex., 1996).
60. Foa, E. B., Hembree, E. A. & Rothbaum, B. O. Prolonged exposure therapy for PTSD, (Oxford University Press, New York, N.Y., 2007).
61. Silverstein, S. M., et al. Development and validation of a World-Wide-Web-based neurocognitive assessment battery: WebNeuro. Behav Res Methods 39, 940-949 (2007).
62. Etkin, A., et al. A cognitive-emotional biomarker for predicting remission with antidepressant medications: a report from the iSPOT-D trial. Neuropsychopharmacology 40, 1332-1342 (2015).
63. Glover, G. H. & Lai, S. Self-navigated spiral fMRI: interleaved versus single-shot. Magn Reson Med 39, 361-368 (1998).
64. Kim, D. H., Adalsteinsson, E., Glover, G. H. & Spielman, D. M. Regularized higher-order in vivo shimming. Magn Reson Med 48, 715-722 (2002).
65. Glover, G. H., Li, T. Q. & Ress, D. Image-based method for retrospective correction of physiological motion effects in fMRI: RETROICOR. Magn Reson Med 44, 162-167 (2000).
66. Shirer, W. R., Ryali, S., Rykhlevskaia, E., Menon, V. & Greicius, M. D. Decoding subject-driven cognitive states with whole-brain connectivity patterns. Cereb Cortex 22, 158-165 (2012).
67. Jackson, T. S. & Read, N. Theory of minimum spanning trees. I. Mean-field theory and strongly disordered spin-glass model. Phys Rev E Stat Nonlin Soft Matter Phys 81, 021130 (2010).
68. Garrison, K. A., Scheinost, D., Finn, E. S., Shen, X. & Constable, R. T. The (in)stability of functional brain network measures across thresholds. Neuroimage 118, 651-661 (2015).
69. Rubinov, M. & Sporns, O. Complex network measures of brain connectivity: uses and interpretations. Neuroimage 52, 1059-1069 (2010).
70. Ghidoni, S., Nanni, L., Brahnam, S. & Menegatti, E. Texture Descriptors Based on Dijkstra's Algorithm for Medical Image Analysis. Stud Health Technol Inform 207, 74-82 (2014).
71. Hawrylycz, M. J., et al. An anatomically comprehensive atlas of the adult human brain transcriptome. Nature 489, 391-399 (2012).
72. Richiardi, J., et al. BRAIN NETWORKS. Correlated gene expression supports synchronous activity in brain networks. Science 348, 1241-1244 (2015).
73. Nievergelt, C. M., et al. Genomic predictors of combat stress vulnerability and resilience in U.S. Marines: A genome-wide association study across multiple ancestries implicates PRTFDC1 as a potential PTSD gene. Psychoneuroendocrinology 51, 459-471 (2015).
74. Fuge, P., et al. Interaction of early life stress and corticotropin-releasing hormone receptor gene: effects on working memory. Biol Psychiatry 76, 888-894 (2014).
75. Tyrka, A. R., et al. Interaction of childhood maltreatment with the corticotropin-releasing hormone receptor gene: effects on hypothalamic-pituitary-adrenal axis reactivity. Biol Psychiatry 66, 681-685 (2009).
76. Hsu, D. T., et al. Variation in the corticotropin-releasing hormone receptor 1 (CRHR1) gene influences fMRI signal responses during emotional stimulus processing. J Neurosci 32, 3253-3260 (2012).
77. Ehlis, A. C., et al. Influence of a genetic variant of the neuronal growth associated protein Stathmin 1 on cognitive and affective control processes: an event-related potential study. Am J Med Genet B Neuropsychiatr Genet 156B, 291-302 (2011).
78. Stjepanovic, D., Lorenzetti, V., Yucel, M., Hawi, Z. & Bellgrove, M. A. Human amygdala volume is predicted by common DNA variation in the stathmin and serotonin transporter genes. Transl Psychiatry 3, e283 (2013).
79. Bradley, R. G., et al. Influence of child abuse on adult depression: moderation by the corticotropin-releasing hormone receptor gene. Archives of general psychiatry 65, 190-200 (2008).
80. Chen, A. C. et al. Causal interactions between fronto-parietal central executive and default-mode networks in humans. Proc Natl Acad Sci USA 110, 19944-19949, doi:10.1073/pnas.1311772110 (2013).
81. Premoli, I. et al. TMS-EEG signatures of GABAergic neurotransmission in the human cortex. J Neurosci 34, 5603-5612, doi:10.1523/JNEUROSCI.5089-13.2014 (2014).
82. Schedlbauer, A. M., Copara, M. S., Watrous, A. J. & Ekstrom, A. D. Multiple interacting brain areas underlie successful spatiotemporal memory retrieval in humans. Sci Rep 4, 6431, doi:10.1038/srep06431 (2014).
83. Kim, H. Dissociating the roles of the default-mode, dorsal, and ventral networks in episodic memory retrieval. Neuroimage 50, 1648-1657, doi:10.1016/j.neuroimage.2010.01.051 (2010).
84. Rossi, S. et al. Prefrontal and parietal cortex in human episodic memory: an interference study by repetitive transcranial magnetic stimulation. Eur J Neurosci 23, 793-800, doi:10.1111/j.1460-9568.2006.04600.x (2006).
85. Sandrini, M., Cappa, S. F., Rossi, S., Rossini, P. M. & Miniussi, C. The role of prefrontal cortex in verbal episodic memory: rTMS evidence. J Cogn Neurosci 15, 855-861, doi:10.1162/089892903322370771 (2003).
86. Turriziani, P., Smirni, D., Oliveri, M., Semenza, C. & Cipolotti, L. The role of the prefrontal cortex in familiarity and recollection processes during verbal and non-verbal recognition memory: an rTMS study. Neuroimage 52, 348-357, doi:10.1016/j.neuroimage.2010.04.007 (2010).
87. Seeley, W. W. et al. Dissociable intrinsic connectivity networks for salience processing and executive control. J Neurosci 27, 2349-2356, doi:10.1523/JNEUROSCI.5587-06.2007 (2007).
88. Shirer, W. R., Ryali, S., Rykhlevskaia, E., Menon, V. & Greicius, M. D. Decoding subject-driven cognitive states with whole-brain connectivity patterns. Cereb Cortex 22, 158-165, doi:10.1093/cercor/bhr099 (2012).
89. Rogasch, N. C., Daskalakis, Z. J. & Fitzgerald, P. B. Cortical inhibition of distinct mechanisms in the dorsolateral prefrontal cortex is related to working memory performance: a TMS-EEG study. Cortex 64, 68-77, doi:10.1016/j.cortex.2014.10.003 (2015).
90. Berlim, M. T. & Van Den Eynde, F. Repetitive transcranial magnetic stimulation over the dorsolateral prefrontal cortex for treating posttraumatic stress disorder: an exploratory meta-analysis of randomized, double-blind and sham-controlled trials. Can J Psychiatry 59, 487-496 (2014).
91. Clark, C., Cole, J., Winter, C., Williams, K. & Grammer, G. A Review of Transcranial Magnetic Stimulation as a Treatment for Post-Traumatic Stress Disorder. Curr Psychiatry Rep 17, 83, doi:10.1007/s11920-015-0621-x (2015).
92. Hill, A. T., Rogasch, N. C., Fitzgerald, P. B. & Hoy, K. E. TMS-EEG: A window into the neurophysiological effects of transcranial electrical stimulation in non-motor brain regions. Neurosci Biobehav Rev 64, 175-184, doi:10.1016/j.neubiorev.2016.03.006 (2016).
93. Chung, S. W., Rogasch, N. C., Hoy, K. E. & Fitzgerald, P. B. Measuring Brain Stimulation Induced Changes in Cortical Properties Using TMS-EEG. Brain Stimul 8, 1010-1020, doi:10.1016/j.brs.2015.07.029 (2015).
94. Rogasch, N. C. & Fitzgerald, P. B. Assessing cortical network properties using TMS-EEG. Hum Brain Mapp 34, 1652-1669, doi:10.1002/hbm.22016 (2013).
95. Kozyrev, V., Eysel, U. T. & Jancke, D. Voltage-sensitive dye imaging of transcranial magnetic stimulation-induced intracortical dynamics. Proc Natl Acad Sci USA 111, 13553-13558, doi:10.1073/pnas.1405508111 (2014).
96. Hipp J F, Hawellek D J, Corbetta M, Siegel M, Engel A K. “Large-scale cortical correlation structure of spontaneous oscillatory activity”, Nature Neuroscience, 15 (6): 884-890; 2013.
97. Hipp J F, Siegel M. “BOLD fMRI Correlation Reflects Frequency-Specific Neuronal Correlation”, Current Biology 25: 1368-1374; 2015.

What is claimed is:

1. A method of treating post-traumatic stress disorder in a subject in need thereof, said method comprising:
(i) determining connectivity between a first cognitive region within the brain of said subject and a second cognitive region within the brain of said subject or determining a complex cognitive behavioral deficiency in said subject; and
(ii) administering a post-traumatic stress disorder treatment to said subject.

2. The method of claim 1, wherein said first cognitive region and said second cognitive region are independently selected from the group consisting of left middle frontal gyrus, left inferior frontal gyrus, left inferior parietal lobule, left middle temporal gyrus, left thalamus, right middle frontal gyrus, right inferior frontal gyrus, right inferior parietal lobule, right dorsomedial PFC, left lateral cerebellum, right caudate, left anterior middle frontal gyrus, left insula, dorsal anterior cingulate cortex (ACC), right anterior middle frontal gyrus, right insula, left lateral cerebellum, right lateral cerebellum, left frontal eye fields, left intraparietal sulcus, left inferior frontal cortex, left inferior temporal gyrus, right frontal eye fields, right intraparietal sulcus, right inferior frontal cortex, right inferior temporal gyrus, right lateral cerebellum, medial prefrontal cortex, left angular gyrus, right superior frontal gyrus, posterior cingulate gyrus, mid-cingulate gyrus, right angular gyrus, thalamus, left hippocampus, and right hippocampus;
wherein said first cognitive region is different from said second cognitive region.

3. The method of claim 1, wherein said determining connectivity is performed using a functional connectivity analysis.

4. The method of claim 3, wherein said functional connectivity analysis is a blood flow analysis.

5. The method of claim 4, wherein said blood flow analysis is an fMRI analysis.

6. The method of claim 4, wherein said blood flow analysis is a near infrared spectroscopy (NIRS) analysis.

7. The method of claim 3, wherein said functional connectivity analysis is an electroencephalogram (EEG) analysis.

8. The method of claim 3, wherein said functional connectivity analysis is a magnetoencephalography (MEG) analysis.

9. The method of claim 1, wherein said determining connectivity comprises administering a transcranial magnetic stimulation (TMS) thereby producing an evoked response.

10. The method of claim 9, wherein said TMS is administered to the right executive control network (ECN).

11. The method of claim 9, wherein said evoked response is monitored by Electroencephalography (EEG).

12. The method of claim 12, wherein an amplitude of the evoked response is measured by EEG at about 30-250 ms after stimulation.

13. The method of claim 1, wherein said determining connectivity is performed using a structural connectivity analysis.

14. The method of claim 13, wherein said structural connectivity analysis is a diffusion-weighted structural connectivity analysis.

15. The method of claim 1 comprising determining a complex cognitive behavioral deficiency in said subject.

16. The method of claim 15, wherein said complex cognitive behavioral deficiency is a memory deficiency.

17. The method of claim 9, wherein said memory deficiency is a long term memory deficiency, a working memory deficiency, a short term memory deficiency, a delayed recall deficiency or an immediate recall deficiency.

18. The method of claim 15, wherein said determining is performed using a card sorting analysis, reward or punishment learning tests, planning test, or navigation test.

19. The method of claim 1, wherein said post-traumatic stress disorder treatment comprises psychotherapy.

20. The method of claim 1 wherein said treatment comprises repetitive transcranial magnetic stimulation (rTMS).

21. The method of claim 20, wherein said rTMS is administered to the right executive control network (ECN).

22. The method of claim 19, wherein said psychotherapy is selected from the group consisting of prolonged exposure therapy, cognitive processing therapy, cognitive behavioral therapy, eye movement and desensitization therapy, acceptance and commitment therapy, and interpersonal psychotherapy.

23. A method of determining connectivity between cognitive regions in a patient suffering from or suspected of suffering from a post-traumatic stress disorder, the method comprising determining connectivity between a first cognitive region within the brain of said subject and a second cognitive region within the brain of said subject or determining a complex cognitive behavioral deficiency in said subject.

24. The method of claim 23, wherein said patient is undergoing a course of treatment for a post-traumatic stress disorder.

25. A system comprising:
at least one processor; and
at least one memory including program code which when executed by the at least one memory provides operations comprising:
determining a connectivity between a first cognitive region and a second cognitive region within a brain of a subject and/or determining a complex cognitive behavioral deficiency in said subject; and
providing, via a user interface, a post-traumatic stress disorder treatment plan for said subject.

26. The system of claim 26, wherein the connectivity between the first cognitive region and the second cognitive region within the brain of the subject and the complex cognitive behavioral deficiency in the subject comprises a biomarker associated with the subject.

27. The system of claim 27, further comprising determining a presence of the biomarker in the subject by at least:
screening the subject for memory deficit; and
performing, on the brain of the subject, an imaging test, when a result of the screening indicates that the subject exhibits memory deficit.

28. The system of claim 27, wherein the imaging test comprises functional magnetic resonance imaging (fMRI) and/or magnetoencephalography (MEG) or electroencephalography (EEG).

29. The system of claim 27, further comprising determining, based on a presence of the biomarker in the subject, the post-traumatic stress disorder treatment plan for the subject.

30. The system of claim 29, wherein psychotherapy is excluded from the post-traumatic stress disorder treatment plan for the subject, when the biomarker is determined to be present in the subject.

31. The system of claim 29, wherein psychotherapy is excluded from the post-traumatic stress disorder treatment plan for the subject, when said subject exhibits abnormal connectivity and complex cognitive behavioral deficiency.

32. The system of claim 29, wherein psychotherapy is included in the post-traumatic stress disorder treatment plan for the subject, when the biomarker is determined to be not present in the subject.

33. The system of claim 29, wherein non-invasive brain stimulation is included the post-traumatic stress disorder treatment plan for the subject, when the biomarker is determined to be present in the subject.

34. The system of claim 30, wherein the treatment plan further includes one or more of psychotherapy, medication and non-invasive brain stimulation.

35. The system of claim 34, wherein the non-invasive brain stimulation is administered based at least in part on the connectivity between the first cognitive region and the second cognitive region within the brain of said subject.

36. The system of claim 25, wherein determining the connectivity between the first cognitive region and the second cognitive region within the brain of the subject comprises evoking a response by administering transcranial magnetic stimulation (TMS).

37. The system of claim 36, wherein the TMS is administered to a right executive control network (ECN).

38. The system of claims 36-37, further comprising monitoring, via Electroencephalography (EEG), the response evoked by the administration of the TMS

39. The system of claim 36, wherein the monitoring of the response evoked by the administration of the TMS comprises measuring, by EEG, an amplitude of the response approximately 30-250 milliseconds (ms) subsequent to the administration of TMS.

40. The system of claim 25, wherein the system is further configured to perform operations comprising the method as recited in any of claims 2-17.

41. A non-transitory computer-readable storage medium including program code which when executed by at least one processor causes operations comprising:
determining a connectivity between a first cognitive region and a second cognitive region within a brain of a subject and/or determining a complex cognitive behavioral deficiency in said subject; and
providing, via a user interface, a post-traumatic stress disorder treatment plan for said subject.

42. The computer-readable storage medium of claim 41, wherein the connectivity between the first cognitive region and the second cognitive region within the brain of the subject and the complex cognitive behavioral deficiency in the subject comprises a biomarker associated with the subject.

43. The computer-readable storage medium of claim 42, further comprising determining a presence of the biomarker in the subject by at least:
screening the subject for memory deficit; and
performing, on the brain of the subject, an imaging test, when a result of the screening indicates that the subject exhibits memory deficit.

44. The computer-readable storage medium of claim 41, wherein the imaging test comprises functional magnetic resonance imaging (fMRI) and/or magnetoencephalography (MEG) or electroencephalography (EEG).

45. The computer-readable storage medium of claim 41, further comprising determining, based on a presence of the biomarker in the subject, the post-traumatic stress disorder treatment plan for the subject.

46. The computer-readable storage medium of claim 43, wherein psychotherapy is excluded from the post-traumatic stress disorder treatment plan for the subject, when the biomarker is determined to be present in the subject.

47. The computer-readable storage medium of claim 43, wherein psychotherapy is excluded from the post-traumatic stress disorder treatment plan for the subject, when said subject exhibits abnormal connectivity and complex cognitive behavioral deficiency.

48. The computer-readable storage medium of claim 43, wherein psychotherapy is included in the post-traumatic stress disorder treatment plan for the subject, when the biomarker is determined to be not present in the subject.

49. The computer-readable storage medium of claim 44, wherein the treatment plan further includes one or more of psychotherapy, medication and non-invasive brain stimulation.

50. The computer-readable storage medium of claim 46, wherein the non-invasive brain stimulation is administered based at least in part on the connectivity between the first cognitive region and the second cognitive region within the brain of said subject.

51. The computer-readable storage medium of claim 39, wherein determining the connectivity between the first cognitive region and the second cognitive region within the brain of the subject comprises evoking a response by administering transcranial magnetic stimulation (TMS).

52. The computer-readable storage medium of claim 49, wherein the TMS is administered to a right executive control network (ECN).

53. The computer-readable storage medium of claim 49, further comprising monitoring, via Electroencephalography (EEG), the response evoked by the administration of the TMS.

54. The computer-readable storage medium of claim 51, wherein the monitoring of the response evoked by the administration of the TMS comprises measuring, by EEG, an amplitude of the response approximately 30-250 milliseconds (ms) subsequent to the administration of TMS.

55. The computer-readable storage medium of claim 41, wherein the operations further comprise the method as recited in claim 1.

56. An apparatus comprising:
means for determining a connectivity between a first cognitive region and a second cognitive region within a brain of a subject and/or means for determining a complex cognitive behavioral deficiency in said subject; and
means for providing a post-traumatic stress disorder treatment plan for said subject.

57. The apparatus of claim 56, wherein the connectivity between the first cognitive region and the second cognitive region within the brain of the subject and the complex cognitive behavioral deficiency in the subject comprises a biomarker associated with the subject.

58. The apparatus of claim 57, wherein the apparatus is further configured to determine a presence of the biomarker in the subject by at least:
screening the subject for memory deficit; and
performing, on the brain of the subject, an imaging test, when a result of the screening indicates that the subject exhibits memory deficit.

59. The apparatus of claim 58, wherein the imaging test comprises functional magnetic resonance imaging (fMRI) and/or magnetoencephalography (MEG) or electroencephalography (EEG).

60. The apparatus of claim 58, wherein the apparatus is further configured to determine, based on a presence of the biomarker in the subject, the post-traumatic stress disorder treatment plan for the subject.

61. The apparatus of claim 60, wherein the apparatus is configured to exclude psychotherapy from the post-traumatic stress disorder treatment plan for the subject, when the biomarker is determined to be present in the subject.

62. The apparatus of claim 60, wherein the apparatus is configured to exclude psychotherapy from the post-traumatic stress disorder treatment plan for the subject, when said subject exhibits abnormal connectivity and complex cognitive behavioral deficiency.

63. The apparatus of claim 60, wherein the apparatus is configured to include psychotherapy in the post-traumatic stress disorder treatment plan for the subject, when the biomarker is determined to be not present in the subject.

64. The apparatus of claim 59, wherein the treatment plan further includes one or more of psychotherapy, medication and non-invasive brain stimulation.

65. The apparatus of claim 61, wherein the non-invasive brain stimulation is administered based at least in part on the connectivity between the first cognitive region and the second cognitive region within the brain of said subject.

66. The apparatus of claim 56, wherein the apparatus is configured to determine the connectivity between the first cognitive region and the second cognitive region within the brain of the subject by at least evoking a response by administering transcranial magnetic stimulation (TMS).

67. The apparatus of claim 69, wherein the TMS is administered to a right executive control network (ECN).

68. The apparatus of claim 66, wherein the apparatus is further configured to monitor, via Electroencephalography (EEG), the response evoked by the administration of the TMS.

69. The apparatus of claim 68, wherein the apparatus is configured to monitor the response evoked by the administration of the TMS by at least measuring, by EEG, an amplitude of the response approximately 30-250 milliseconds (ms) subsequent to the administration of TMS.

70. The apparatus of claim 56, further comprising means for performing the method as recited in claim 1.

71. A method of treating post-traumatic stress disorder in a subject in need thereof, said method comprising:
determining a connectivity between a first cognitive region within a brain of said subject and a second cognitive region within the brain of said subject and/or determining a complex cognitive behavioral deficiency in said subject; and
providing, via a user interface, a post-traumatic stress disorder treatment plan for said subject.

72. The method of claim 71, wherein the connectivity between the first cognitive region and the second cognitive region within the brain of the subject and the complex cognitive behavioral deficiency in the subject comprises a biomarker associated with the subject.

73. The method of claim 72, further comprising determining a presence of the biomarker in the subject by at least:
screening the subject for memory deficit; and
performing, on the brain of the subject, an imaging test, when a result of the screening indicates that the subject exhibits memory deficit.

74. The method of claim 72, wherein the imaging test comprises functional magnetic resonance imaging (fMRI) and/or magnetoencephalography (MEG) or electroencephalography (EEG).

75. The method of claim 72, further comprising determining, based on a presence of the biomarker in the subject, the post-traumatic stress disorder treatment plan for the subject.

76. The method of claim 75, wherein psychotherapy is excluded from the post-traumatic stress disorder treatment plan for the subject, when the biomarker is determined to be present in the subject.

77. The method of claim 75, wherein psychotherapy is excluded from the post-traumatic stress disorder treatment plan for the subject, when said subject exhibits abnormal connectivity and complex cognitive behavioral deficiency.

78. The method of claim 75, wherein psychotherapy is included in the post-traumatic stress disorder treatment plan for the subject, when the biomarker is determined to be not present in the subject.

79. The method of claim 76, wherein the treatment plan further includes one or more of psychotherapy, medication and non-invasive brain stimulation.

80. The method of claim 79, wherein the non-invasive brain stimulation is administered based at least in part on the connectivity between the first cognitive region and the second cognitive region within the brain of said subject.

81. The method of claim 71, wherein determining the connectivity between the first cognitive region and the second cognitive region within the brain of the subject comprises evoking a response by administering transcranial magnetic stimulation (TMS).

82. The method of claim 81, wherein the TMS is administered to a right executive control network (ECN).

83. The method of claim 81, further comprising monitoring, via Electroencephalography (EEG), the response evoked by the administration of the TMS.

84. The method of claim 83, wherein the monitoring of the response evoked by the administration of the TMS comprises measuring, by EEG, an amplitude of the response approximately 30-250 milliseconds (ms) subsequent to the administration of TMS.

85. The method of claim 71, further comprising the method as recited in claim 1.

86. A method of treating a subject having or suspected of having inhibitory right ECN post-traumatic stress disorder, the method comprising administering TMS.

87. The method of claim 86, wherein TMS is administered to the right ECN.

88. The method of claim 86, wherein TMS is delivered repetitively in a pattern that is intended to induce plasticity.

89. The method of claim 86, wherein TMS is rTMS.

90. The method of claim 89, wherein rTMS comprises stimulation at greater than 5 Hz.

91. The method of claim 89, wherein rTMS comprises stimulation at less than or equal to 1 Hz.

92. The method of claim 89, wherein rTMS comprises either a continuous or an intermittent theta burst pattern.


SEQUENCE LISTING (5 sequences)

SEQ ID NO: 1 - DNA, 2681 bp, Artificial Sequence
  Synthetic nucleotide
ggcgaggccggcaagaggcggccgcgggccgggctgcgtcgggaaacggcggccagacttccccgggaaggggcgagcgagagccgggccgggccgggccgggccgcggggccgggaagcgccgagccgggcatctcctcaccaggcagcgaccgaggagcccggccgcccaccccgtgccgcccgagcccgcagccgcccgccggtccctctgggatgtccgtaggacccgggcattcaggacggtagccgagcgagcccgaggatgggagggcacccgcagctccgtctcgtcaaggcccttctccttctggggctgaaccccgtctctgcctccctccaggaccagcactgcgagagcctgtccctggccagcaacatctcaggactgcagtgcaacgcatccgtggacctcattggcacctgctggccccgcagccctgcggggcagctagtggttcggccctgccctgcctttttctatggtgtccgctacaataccacaaacaatggctaccgggagtgcctggccaatggcagctgggccgcccgcgtgaattactccgagtgccaggagatcctcaatgaggagaaaaaaagcaaggtgcactaccatgtcgcagtcatcatcaactacctgggccactgtatctccctggtggccctcctggtggcctttgtcctctttctgcggctcaggccaggctgcacccattggggtgaccaggcagatggagccctggaggtgggggctccatggagtggtgccccatttcaggttcgaaggagcatccggtgcctgcgaaacatcatccactggaacctcatctccgccttcatcctgcgcaacgccacctggttcgtggtccagctaaccatgagccccgaggtccaccagagcaacgtgggctggtgcaggttggtgacagccgcctacaactacttccatgtgaccaacttcttctggatgttcggcgagggctgctacctgcacacagccatcgtgctcacctactccactgaccggctgcgcaaatggatgttcatctgcattggctggggtgtgcccttccccatcattgtggcctgggccattgggaagctgtactacgacaatgagaagtgctggtttggcaaaaggcctggggtgtacaccgactacatctaccagggccccatgatcctggtcctgctgatcaatttcatcttccttttcaacatcgtccgcatcctcatgaccaagctccgggcatccaccacgtctgagaccattcagtacaggaaggctgtgaaagccactctggtgctgctgcccctcctgggcatcacctacatgctgttcttcgtcaatcccggggaggatgaggtctcccgggtcgtcttcatctacttcaactccttcctggaatccttccagggcttctttgtgtctgtgttctactgtttcctcaatagtgaggtccgttctgccatccggaagaggtggcaccggtggcaggacaagcactcgatccgtgcccgagtggcccgtgccatgtccatccccacctccccaacccgtgtcagctttcacagcatcaagcagtccacagcagtctgagctggcaggtcatggagcagcccccaaagagctgtggctggggggatgacggccaggctccctgaccaccctgcctgtggaggtgacctgttaggtctcatgcccactcccccaggagcagctggcactgacagcctgggggggccgctctccccctgcagccgtgcaggactctagctcatgagtggaaagtcacctacaggactgggccgggcccagggcctctggcttccctgcccaatcctccctggagaagggacatgggaatgaattgaaatggggcgctggacacctacagcagcacgcatgtccctccaaggctgtcttctcccagagcacaagaaggccagcccactgggccctggggctgccctcggcaaccgtggggaggccatttgctgccctggggcatcatgggcaactcgtgacagcctctgactcaccacgatgacgcctctggacctcggtgatgccttccgacaccactgggaaccaagggccctcactcaggaaccctggagacagaagtcaggtgtcatcatcagacttgcggccacagcactagagtcacccccccaggcctccagaaccttactggcactgtggcactgccaccagcaatgccctgccttgctgccttcaccctgaacatttagtaccctgcaggccaggccagcttcccctcacttaaccaccccataccagtcacctcctgctccttttcctcttttgtgagaagatgggggctggagggggcagagtggcctgtgagcaagagccaggggtgtcccagtcccagcctctggggcagagcttgtagccctggatggcctctggggcaggaccactagctaagcaagccaggagaagacccctgcccaagtggctcttgggacaacgtgctgcttacactccaggtgtggaccggccgcagcccccactgacctgcccatgtccagagggactggacagccagggcagggctttggggggcactagaagatgagggtgtcggctgtgaggcgggtggctggtataaataatatttatcttttcaaccagcaaaaaaaaaaaaaaaaaa

SEQ ID NO: 2 - DNA, 2993 bp, Artificial Sequence
  Synthetic nucleotide
ggggaggggggccttctcatgtctgcaaccctctgcccatgcagtctgtgccaccccccacccctggtcacctggtcaactctgcattcttcagaggccagttccagggcccgtctgcttagtagtacctgcccaactctccaaagcaaagcagctcagttctccctccaatgtaccccttttcccagtgcactctgtacactcactggaccctgtgcccgtgaggctcacaagtagagcacttagaccagcctaagtgcacgaaaaccacctcggcacaaagcacttaggacccaagacactaattagatggcaaatatacaggctataagttcttattctcgcatcttaatgtcctggcgtgcacagaggagcagagcaaatcttgattcgtcagaaaatgaagccaaattccaggcaaagggctagtcttcccttggctgcctagaaccctgacacttgtgtccctctcttgaatgtgatggttcacacaggcattttctaaacacagaggactggtgttggttatgcaaagaaaaatgcttcttaaaattcccaaaccaacctttcctctctggaaaggtctgctctgtagcagaatctgtgcctgtgccacctcttgctctgtggaaggtttgtgagggcttctgccttattcctgaatcatacttccatgaaaagtccagagatttcctgcatttttcctcccaccatccgctgatctcctcttcaccatccgctgatctcctcttcaagctccactgtggttttctatctccatcaataggcaattactgcactggcaggaacaggcccccaaagtttcaaggatgttgtttgtttgcccttaaaatggagaatttttttgtttgtttgttttttgagacagtcttgctctgtcgcccaggctagagtgcagtggtgcgatctccgctcactccaacctctgtctcctgggttcaagcaattctcctgcctcagcctcccaagtagctgggattacaggcctgcaccaccatgcccagctaagttttgtgttttcagtagagatgaggtttcaccctgttggccaggctggtctcaaactcctgacttcaagtgatcagcccgcctcggcctcccaaagtgctgggattacaggagtgagccaccatgctgggctaaaacaacaaaaaacaaaacaaacaaaaaaaaaaacacggagaattcttaacctggggagcgggcagtgtgaagatttgcaaataaatgcagttggtgtgtgtgatgaaaaccattttcccggggagggttcttggcttcctccacttctcaccaacaactagcaccctgaaacagtgggaactacagtcgaagcatatggtaaatggggtgaactgaacatgtcatcttctctgaaatcaaaccaaagtgcttttggcctttctcagatgcctcttgctcctccactccatcaccagtcccagccactggcgctcctttcagaaccaagttttcaagacttcatgtgatttactatcttccaactagtagagaaaatcaaaacggcactgaagctgaagctggcagagcacgaggttcactcactggctaatgcctcacttccgtgcacagcctggcagccccaaggctccaaataaaacttgcagctgagctacaaatctctccactcaccaaagagccaaaccttcaaggttaaagtctgaggccaagggtgcgtgcggctcaattatagcatcgcgtaaaaggattttccaaagatctagtgggtccctgggatggaagggaaagcagactgggcgggtatcatgctcccattttaagtctggggtagttgatgtacccaatgtcacataactgtttttaggagctgggattcagtaagcaggtgctcctgctgtctttcaaggagatgaaccatgaagacagtcgcgccttctggctggccaggcgcaggtcaagacagcgccttggaagaagggatgggcagtggggaggaaggtggtgcaggagcctgtggagggggtggagggccaggttctgacatggcctgctccagcctgcagcagctgctacccacacccccacccccagggccagcacagaatggggctggaatgaagctgccatcccaattcccagtgtgattttggtacaaaggttaatcacacccagaaaatgcttatcagagaccataaataaccgtgttgttgaagctcagtttggtctttaaggcacaaaagtgcaatttcctttgatgtgatgattcggtaattaaccttttggggagggtcctgaaggccactggagggagggaggggtggcctggaggcagctgaggcatctcaggcctggttcctacaagttcctggggaacagggaaaagggagcctgttttctctctcctttcacagctagcactgatgctcagcttgggagagtccccagagcccccaggggtcagggaccgcactggccttcggcagagccacagctggacagccatccttcaacacggtccctcgcccttctgctgagccccaaggcagtcctgtcactggctgtgtggctttcggtgagtcacctgactcctctgagactcaacatcctcatctgtgaagtgggggcaatgtggtctttcttacccacttcactgggagatggaccatttgtggggaaccaggagacctgcatctgcaatataaagtccctacaagcatcggcagggtcatcagcccagcttggctgttctgggccccagaatgcacctccactgcagagggctggcctggagcagctttcctgatgtcccttccgggccctgggggaggccccgcaggctggacctggcctctcctcatcttccgccaggtccttctgccttctcaggaagccttgctttgggaaggagtttttccatttaaaaggtaagttgctttttaggtttgaaatccactggattgagtggtgtctccaacccccttggtggtctg

SEQ ID NO: 3 - DNA, 1000 bp, Artificial Sequence
  Synthetic nucleotide
aaggtgaggttcaagaggggttgagggtatgctcatccctgtggctaccagtacccacccctagccgggacatgaccaggcaccttctgtgagccagcacagggacccagtgtgccttctccaagtccagggcttgttcctctgcccccaggctctccctagtgaaggaacagctggccaggaagcagctaccatgagatgtggaggcctcctgttcccacagagccaccttcagaaatgaaggcctcctgcccgcctgcagacacttgcgtctgtttctttgcaagcatgcgtcttccactctgcctcatcgcacatgcatttgtggattcgatgcccagactagcctgtgagcccccttgtttcccccagtgccctgcgtggggcctgctgtgtagtaggtgcttcatgaaagagttggtgtgaatcaaggagaggcttgaagacttaaatagaaggtccacaagccttccaaagacactcaggtgcagggaccctctcatttttgcccagcagcagccatgcccaggaccacacccaaagttttaaaagacatgccctaggcagcccaaacacctagatttgggtcccagtcactgccatgtactggctgcatggtgccatgggggttatgtaactctcccatgcctcagtttcctcaactgtacaaacgttcggctttacactcttgtgaggatgaaacgagatgattatgcaaaagcactctgtaaatggtaaagtgacacagatattaataatatttgcagctatgctgtcttttctattggagagatacagagcacggcgctggagaccacaaccagaagctgggctgcccgtgctccactcagggctctggcgcttactagttctctgtgcctcagttttcttatgtgtaaaatgggggcaacagcacctacttcagagggacattgtgcaaattgggttaatacagctccagtccttgatcagtgcctggcacgggttagcaccgtggagt

SEQ ID NO: 4 - DNA, 2251 bp, Artificial Sequence
  Synthetic nucleotide
gctctcggccaatgcggagccccgcgcggaggtcacgtgcctctgtttggcgcttttgtgcgcgcccgggtctgttggtgctcagagtgtggtcaggcggctcggactgagcaggactttccttatcccagttgattgtgcagaatacactgcctgtcgcttgtcttctattcaccatggcttcttctgatatccaggtgaaagaactggagaagcgtgcctcaggccaggcttttgagctgattctcagccctcggtcaaaagaatctgttccagaattccccctttcccctccaaagaagaaggatctttccctggaggaaattcagaagaaattagaagctgcagaagaaagacgcaagtcccatgaagctgaggtcttgaagcagctggctgagaaacgagagcacgagaaagaagtgcttcagaaggcaatagaagagaacaacaacttcagtaaaatggcagaagagaaactgacccacaaaatggaagctaataaagagaaccgagaggcacaaatggctgccaaactggaacgtttgcgagagaagatgtacttctggactcacgggcctggggcccacccagcacagatctctgctgagcaatcttgtctccactctgttcctgccctttgcccagccctgggcctccaatctgcattgattacctggtctgatctctctcaccatcactaggtacttaataaatatttgctgttgatgatagcaatgaccttgagactgatgaacagtctggccaagaggatccttgatgtggaagatagaaaaagcctttggggtcaggcagacttggattctaataccagccagttctgcttgctgtgtctgagcctcagtttactcatctgtgaagaggaggtagcaagaatgaaaatgcctgccttgtggtttgttgtaaggacagacactgccaacgtagagggcccagcagctcacagaccagttgctctgagagcagaccactcttgccttgatggtagggaactatttttgtgcgtggcaagtgggaccttaggaaggaaggcaactgtgaggcttctgagaaggaccctacacaagggagtttcctcccagggcaggtgaatggagagggtggcagaagcctacgggaaggggtcacagggatcagctagagagtgccaccacccttcctggggaatgcagggcaaggtccctggtgggagttttcctgggaagccaaagaagcgcccaacaaagacagaatcaacatttgggtacctttggtacccagaggcagcaatgccaactacaaccacactggaagaagaagaccttctccgcatagattctctgatctcttcctccttcatggcaccagccctggggaaccagcatggtggggaaataatgaagctggaatacaaccacttacagacttcacaacctcctcctgtagataccaaagggattttaggatcacattttatttctcacctgagcaagaaaagctacaggagcatctcaagcagagggcaggagtctccagaggagttcaaggggctctggcaagaaaaatcaaggggctgtgttcaagaactggctcccttggtgattgtattacgaagcccatgtgtgctggatgctgatgaaattgctgccaaatgcctgtgcagccttggcaaggccctttatttctctgggtctccatttctctctctcttttttttttttttttttttttgaggcagagtctcactctgtcgcccaggctggagggcagtggcgtgatctcggctcactgcaagccccaccttctgagttcacgccattctactgcctcagcctcccgagtagctgggactacaggcgcccaccaccacgcccggcttattttttgtatttttagtagagacggggtttcaccgcattagccaagatggtctcgatctcctgacctcgtgattcacccacctcagcctcccaaagtgctgggattacaggcatgagccactgcgcccggcctgggtctccgtttctctagctgtgaaatgactgttctaaaagagccctgccggactttggcagtctgtaagaagacctgagttcttctctcagttccaagcaggaaaattgaacataccctgagcccagagcctgcaacaaactctgggcagcctcaggaagtcaggcagtgaagtcggaaaaatgatctcttctgtatagggagaaaataaaagtttaaaaatttgtaaaaaaaaaaaaaa

SEQ ID NO: 5 - DNA, 1000 bp, Artificial Sequence
  Synthetic nucleotide
cttcagaagagagtggatcttgaagttcgttttaggaagctttcgagaagggcgattctgcccctctccaaggtgaagtggcaaaactgttgtctctccttacagttgaaagcccttcctattccaaggcttatataggacacccacaaaagctgacaataatgagctgtgatctggctcttgggaaggcagccccaccctaaagaacaaggatcagactgggtgtggtggctcatgcctgtaatcccagcactttgggaggctgagacaagaggattgcttgaacccaggagttcgaggccaacctgggcaacatagtgagaccctgtctcaattttttttaagtcaaataataataataataataataataataataataataataataataataaaaggacaggaatcccacctcaaaagcacctttttattcaaaggctcattctaggggaaactttaacccagccctctcagtcctcaagcagctgtgaggagctggaacattgtcacagactggcagaaccaggcatctcccaacaaacccagaagccaagtggcattgctaaaaaagattaaaaatgaacatgagttctaacatctacgttccttgctatggtttccatggaaatagacatctgctctagggagaaagaaaatgtggggggaagcagtgtaagtgctttcaaattatcagtgttttgaaagatttatttcttttttctgtcacaacattaatagtcaggaatgtgagacttttgtgtggcaatttattgtacttaaaaaaatgcattaaatgagtgggaccttcctctgtgaacctccagatcaaatacaaagacttcaagataaaaagcctgttcagtgggtaaggacctcttgggcttgggttccttgctcatttgtgttgcctgcctatctgtcgctttcctgaggggtctcatgcccatccttctccaacttttctaatgatgtctgagattctgttcagaaaaatgac